(19) United States
(12) Patent Application Publication
(10) Pub. No.: US 2016/0362710 A9
(43) Pub. Date: Dec. 15, 2016

(54) SYSTEMS AND METHODS FOR PRODUCTION OF MIXED FATTY ESTERS

(71) Applicant: REG Life Sciences, LLC, Ames, IA (US)

(72) Inventors: Grace J. LEE, South San Francisco, CA (US); Zhihao HU, South San Francisco, CA (US)

(73) Assignee: REG LIFE SCIENCES, LLC, South San Francisco, CA (US)

(21) Appl. No.: 14/599,438

(22) Filed: Jan. 16, 2015

Related U.S. Application Data
(63) Continuation of application No. 12/545,001, filed on Aug. 20, 2009, now abandoned.
Application No. 12/545,001 is a continuation-in-part of application No. 12/543,419, filed on Aug. 18, 2009, now abandoned.
(60) Provisional application No. 61/089,806, filed on Aug. 18, 2008.

(51) Int. Cl. C12P 7/64; C10L 1/02; C07C 69/34; C07C 69/587; C07C 69/533

(52) U.S. Cl. CPC C12P 7/649; C07C 69/587; C07C 69/533; C07C 69/34; C10L 1/026; C10L 2290/26; C10L 2200/0476

(57) ABSTRACT

Disclosed herein are various embodiments regarding the production of fatty acid methyl esters. Disclosed herein are various embodiments regarding the use of methanol compositions for the production of fatty esters.

### REFERENCE TO RELATED APPLICATION

This application is a continuation-in-part of U.S. patent application Ser. No. 12/543,419, filed Aug. 18, 2009, entitled Systems and Methods for Production of Mixed Fatty Esters (Attorney Docket No. LS9.006A1), which claims the priority benefit of provisional application No. 61/089,806, filed Aug. 18, 2008, the entirety of which is incorporated herein by reference.

### FIELD OF THE INVENTION

The present disclosure relates generally to compositions and methods for producing mixtures of fatty esters.

### BACKGROUND

Fuel sources are becoming increasingly limited and difficult to acquire. As a result, efforts have been directed toward harnessing sources of renewable energy, such as sunlight, water, wind, and biomass. The use of biomasses to produce new sources of fuel which are not derived from petroleum sources, (e.g., biofuel, such as biodiesel) has emerged as one alternative option. Current methods of making biodiesel involve transesterification of triacylglycerides (e.g., vegetable oil) which leads to a mixture of fatty esters and glycerin.

As demand for biofuels grow, there is a continuing need for new biofuels and for methods and systems of ecomonically producing the biofuels.

### SUMMARY OF THE INVENTION

The present disclosure provides fatty acid ester compositions and systems and methods for producing fatty acid methyl esters, which can be utilized as a biofuel (e.g., a biodiesel).

In some aspects, the invention comprises a method of producing a fatty acid methyl ester.

The method comprises providing a fatty ester production host. The method further comprises providing methanol to the fatty ester production host. The method can further comprise converting the methanol to a fatty acid methyl ester using the fatty ester production host.

In some aspects, the fatty ester production host comprises a heterologous nucleic acid sequence encoding an ester synthase. In some embodiments, the ester synthase is atfA1, wax-dgat, or mWS.

In some embodiments, the fatty ester production host comprises a heterologous nucleic acid sequence encoding a thioesterase. In some embodiments, the thioesterase is tesA, ′tesA, tesB, fatB, fatB2, fatB3, fatB [M141T], fatA, or fatA1.

In some embodiments, the fatty ester production host comprises a heterologous nucleic acid sequence encoding an acyl-CoA synthase. In some embodiments, the acyl-CoA synthase is: fadD, fadK, BH3103, yhfL, Pfl-4354, EAV15023, fadD1, fadD2, RPC_4074, fadDD35, fadDD22, faa3p, or a gene encoding ZP_01644857.

In some embodiments, the fatty ester production host either lacks a nucleic acid sequence encoding for an acyl-CoA dehydrogenase or expresses an attenuated level of an acyl-CoA dehydrogenase.

In any of the aspects described herein, the host cell can be selected from the group consisting of a mammalian cell, plant cell, insect cell, yeast cell, fungus cell, filamentous fungi cell, and bacterial cell.

In some embodiments, the host cell is a Gram-positive bacterial cell. In other embodiments, the host cell is a Gram-negative bacterial cell.

In some embodiments, the host cell is selected from the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

In particular embodiments, the host cell is a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell.

In other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, or a Mucor michei cell.

In yet other embodiments, the host cell is a Streptomyces lividans cell or a Streptomyces murinus cell. In other embodiments, the host cell is an Actinomycetes cell.

In some embodiments, the host cell is a CHO cell, a COS cell, a VERO cell, a BHK cell, a HeLa cell, a Cv1 cell, an MDCK cell, a 293 cell, a 3T3 cell, or a PC12 cell.

In particular embodiments, the host cell is an E. coli cell, such as a strain B, a strain C, a strain K, or a strain W E. coli cell.

In other embodiments, the host cell is a cyanobacterial host cell.

In some embodiments, the fatty ester production host produces fatty acid methyl esters at a titer of about 50 mg/L or more, about 100 mg/L or more, about 150 mg/L or more, about 200 mg/L or more, about 250 mg/L or more, 300 mg/L or more, about 350 mg/L or more, about 400 mg/L or more, about 450 mg/L or more, or about 500 mg/L or more.

In some embodiments, the fatty ester production host has a specific productivity for fatty esters of about 5, about 10 mg/L/OD₆₀₀ or more, about 15 mg/L/OD₆₀₀ or more, about 20 mg/L/OD₆₀₀ or more, about 25 mg/L/OD₆₀₀ or more, about 30 mg/L/OD₆₀₀ or more, about 35 mg/L/OD₆₀₀ or more, about 40 mg/L/OD₆₀₀ or more, about 45 mg/L/OD₆₀₀ or more, or about 50 mg/L/OD₆₀₀ or more.

In some embodiments, the fatty acid methyl ester has the following formula:

BCOOCH₃

wherein B is a carbon chain that is at least 6 carbons in length.

In some embodiments, the B carbon chain is at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 carbons in length. In other embodiments, B has a number of carbon atoms independently selected from the group consisting of: 6, 7, 8, 9, 10, 11, 12, 13, 14, 15, 16, 17, 18, 19, 20, 21, 22, 23, 24, 25, 26, 27, 28, 29, and 30.

In some embodiments, the fatty ester production host produces a fatty acid methyl ester composition comprising more than one fatty acid methyl ester, wherein the fatty acid methyl ester composition comprises at least a first fatty acid methyl ester having the following formula:

B₁COOCH₃

and a second fatty acid methyl ester having the following formula:

B₂COOCH₃

wherein B₁ is a carbon chain that is at least 6 carbons in length, wherein B₂ is a carbon chain that is at least 6 carbons in length, and wherein B₁ and B₂ are not the same.

In some embodiments, the methanol is provided at a concentration of about 0.1% (v/v) or more, about 0.2% (v/v) or more, about 0.3% (v/v) or more, about 0.4% (v/v) or more, about 0.5% (v/v) or more, about 1% (v/v) or more, about 1.5% (v/v) or more, about 2% (v/v) or more, about 2.5% (v/v) or more, about 3% (v/v) or more, about 3.5% (v/v) or more, about 4% (v/v) or more, about 4.5% (v/v) or more, about 5% (v/v) or more, about 5.5%, about 6% (v/v) or more, about 6.5% (v/v) or more, or about 7% (v/v) or more.

In other embodiments, the methanol is provided at a concentration of about 0.1%, about 0.2%, about 0.3%, about 0.4%, about 0.5%, about 1%, about 1.5%, about 2%, about 2.5%, about 3%, about 3.5%, about 4%, about 4.5%, about 5%, about 5.5%, about 6%, about 6.5%, or about 7% (v/v) or less.

In some embodiments, converting the methanol comprises performing a fermentation.

In some embodiments, converting the methanol produces a product stream, the method further comprising performing a separation process to extract the fatty acid methyl ester from the product stream.

In some embodiments, the separation process is chosen from the group consisting of a filtration, a distillation, and a phase separation process.

In some embodiments, the method further comprises administering a production substrate to the fatty ester production host, wherein the production substrate is utilized by the fatty ester production host to produce fatty acid methyl esters.

In another aspect, the invention comprises a fatty ester composition. In some embodiments, the fatty ester composition comprises a production host.

In some embodiments, the fatty ester composition further comprises a fatty acid methyl ester having the following formula:

BCOOCH₃

wherein B is a carbon chain that is at least 6 carbons in length.

The fatty ester composition of claim 71, wherein B₁ and B₂ carbon chains have a number of carbon atoms independently selected from the group consisting of: 6, 7, 8, 9, 10, 11, 12, 13, 14, 15, 16, 17, 18, 19, 20, 21, 22, 23, 24, 25, 26, 27, 28, 29, and 30.

In some embodiments, the B side carbon chain is unsaturated, monounsaturated, or polyunsaturated.

In some embodiments, the fatty acid methyl ester is secreted from by the fatty ester production host.

In certain embodiments, the host cell overexpresses a nucleic acid sequence that encodes an enzyme described herein. In some embodiments, the method further includes transforming the host cell to overexpress a nucleic acid sequence that encodes an enzyme described herein.

In certain embodiments, the host cell overproduces a substrate described herein. In some embodiments, the method further includes transforming the host cell with a nucleic acid sequence that encodes an enzyme described herein, and the host cell overproduces the product of the enzyme described herein. In other embodiments, the method further includes culturing the host cell in the presence of at least one substrate described herein, which may be overproduced. In some embodiments, the substrate is a fatty acid derivative, an acyl-ACP, a fatty acid, an acyl-CoA, or a fatty ester.

In some embodiments, the fatty acid derivative substrate is an unsaturated fatty acid derivative substrate, a monounsaturated fatty acid derivative substrate, or a saturated fatty acid derivative substrate. In other embodiments, the fatty acid derivative substrate is a straight chain fatty acid derivative substrate, a branched chain fatty acid derivative substrate, or a fatty acid derivative substrate that includes a cyclic moiety.

In some embodiments, the fatty acid methyl ester is a straight chain ester, a branched chain ester, or a cyclic ester.

In some embodiments, the biological substrate is a fatty acid derivative, an acyl-ACP, a fatty acid, an acyl-CoA, or a fatty ester.

In another aspect, the invention features a fatty acid methyl ester produced by any of the methods or microorganisms described herein. In particular embodiments, the fatty acid methyl ester has a δ¹³C of about −15.4 or greater. For example, the fatty acid methyl ester has a δ¹³C of about −15.4 to about 10.9, for example, about −13.92 to about 13.84. In other embodiments, the fatty acid methyl ester has an f_{M}¹⁴C of at least about 1.003. For example, the fatty acid methyl ester has an f_{M}¹⁴C of at least about 1.01 or at least about 1.5. In some embodiments, the fatty acid methyl ester has an f_{M}¹⁴C of about 1.111 to about 1.124.

In another aspect, the invention features a biofuel that includes a fatty acid methyl ester produced by any of the methods or microorganisms described herein. In particular embodiments, the fatty acid methyl ester has a δ¹³C of about 15.4 or greater. For example, the fatty acid methyl ester has a δ¹³C of about −15.4 to about 10.9, for example, about −13.92 to about 13.84. In other embodiments, the fatty acid methyl ester has an f_{M}¹⁴C of at least about 1.003. For example, the fatty acid methyl ester has an f_{M}¹⁴C of at least about 1.01 or at least about 1.5. In some embodiments, the fatty acid methyl ester has an f_{M}¹⁴C of about 1.111 to about 1.124. In some embodiments, the biofuel is biodiesel.

There are additional features and advantages of the subject matter described herein. They will become apparent as this specification proceeds.

In this regard, it is to be understood that this is a brief summary of varying aspects of the subject matter described herein. The various features described in this section and below for various embodiments can be used in combination or separately. Any particular embodiment need not provide all features noted above, nor solve all problems or address all issues in the prior art noted above.

### BRIEF DESCRIPTION OF THE DRAWINGS

Certain embodiments will be described in more detail with reference to the following drawings:

FIG. 1 is a flow chart depicting one embodiment of one of the disclosed methods.

FIG. 2 shows the FAS biosynthetic pathway.

FIG. 3 shows biosynthetic pathways that produce fatty esters.

FIG. 4 shows biosynthetic pathways that produce fatty alcohols.

FIG. 5 shows biosynthetic pathways that produce fatty esters.

FIG. 6 shows a table that identifies examples of various genes that can be over-expressed or attenuated to increase fatty acid derivative production in various embodiments.

FIG. 7 is a graph depicting the fatty esters produced from a mixed alcohol experiment.

FIG. 8 depicts the GC/MS results for a mixed alcohol fatty ester production.

FIG. 9A is a graph depicting the fatty ester titers for a 30° C. experiment.

FIG. 9B is a graph depicting the fatty ester titers for a 37° C. experiment.

FIG. 10A is a graph comparing the amount of saturated and unsaturated fatty ester produced.

FIG. 10B is a graph comparing the amount of saturated and unsaturated fatty ester produced.

FIG. 10C is a graph depicting the fatty ester titers for a 30° C. experiment.

FIG. 10D is a graph depicting the percent acyl composition for a 30° C. experiment.

FIG. 11A is a graph comparing the amount of saturated and unsaturated fatty ester produced.

FIG. 11B is a graph comparing the amount of saturated and unsaturated fatty ester produced.

FIG. 11C is a graph depicting the fatty ester titers for a 37° C. experiment.

FIG. 11D is a graph depicting the percent acyl composition for a 37° C. experiment.

FIG. 12 is a graph comparing the saturation of the fatty esters produced for various combinations of starting alcohols.

FIG. 13 is a graph depicting the percent of saturated and unsaturated product for various combinations of alcohols.

FIG. 14 is a graph depicting the amount of alkyl ester produced from various starting alcohols.

FIG. 15 is a graph depicting the relative amounts of fatty esters produced by a fatty ester production host from methanol, ethanol, and methanol:ethanol mixtures.

FIG. 16 is a graph depicting the specific productivity of FAME and FAEE produced by a fatty ester production host when fed methanol, ethanol, or methanol:ethanol mixtures.

FIG. 17 is a graph depicting the titers of FAME produced by a fatty ester production when fed different concentrations of methanol.

FIG. 18 is a graph depicting the specific productivity of FAME produced by a fatty ester production when fed different concentrations of methanol.

FIG. 19 is a diagram illustrating the cloning methods used to generate the integration fragment lacZ::′tesA fadD atfA1.

FIG. 20 is a graph depicting the specific productivity of FAME produced by a fatty ester production when fed different concentrations of methanol.

Various Figures showing graphs are provided with a legend identifying the bars shown in the graphs. It will be appreciated that the identities, in order as listed from top to bottom, correspond to the bars, in order as extending left to right, for each set of data identified on the X-axis, unless otherwise indicated herein.

### DETAILED DESCRIPTION OF SOME PREFERRED EMBODIMENTS

The use of fatty esters in or as a fuel is becoming more desirable as the need for renewable fuels increases. One method of producing fatty esters involves the use of a biological production host to convert a specific alcohol into a specific fatty ester. People have used such production hosts to produce a specific fatty ester, which can then, optionally, be incorporated or modified into a biofuel. Of course, the properties of the fatty ester produced will depend upon the specific molecular structure of the fatty ester itself, such as degree of unsaturation and the length of the various carbon chains. Furthermore, fuel properties such as cloud point, cetane number (CN), viscosity, and lubricity can change considerably depending on the alcohol moiety incorporated into the fatty ester. (See, generally, Gerhard Knothe, “‘Designer’ Biodiesel: Optimizing Fatty Ester Composition to Improve Fuel Properties,” Energy & Fuels, 22:1358-1364 (2008)).

In situations where the properties of a single specific fatty ester are ideal for its use, relatively little additional manipulation may need to occur in order to use the fatty ester as or in a fuel. However, in situations where the inherent properties of the specific fatty ester are not ideal, subsequent manipulation of the composition is usually required so that a final product with the desired properties is achieved. The present Inventors have appreciated that one way in which the properties of a fatty ester composition can be manipulated is through the use of a combination of different fatty esters (e.g., fatty esters having different degrees of unsaturation or chain lengths) so that the resulting fatty ester mixture has the desired properties. Such a goal can be achieved by producing the various fatty ester compositions, via the production hosts, and then combining the fatty ester products to achieve the desired fatty ester composition.

While post production combination is one way for obtaining a desired fatty ester mixture, the present Inventors have further appreciated that the production of fatty esters in production hosts provides an opportunity to tailor the final fatty ester mixture through various, earlier, biological manipulations. Among other things, the present Inventors have appreciated that the customization process for producing a desired fatty ester mixture can begin before any fatty ester is present. In particular, by selecting various types and/or amounts of alcohols to combine with a fatty ester production host, one can make a variety of fatty esters concurrently. This in turn allows one to obtain a desired final combination of fatty esters, with desirable properties as a mixture (such as desired cloud point, cetane number, viscosity and lubricity), and can eliminate or reduce the need for subsequent manipulation of the the fatty ester product in its adaptation to a fuel.

In some embodiments, the above method can be applied for the production of a customized fatty ester mixture or fuel component. In some embodiments, a desired fatty ester profile can be identified (for example, a fatty ester composition having a high cetane number and a low melting point) and an appropriate fatty ester mixture for that fatty ester profile can be determined. One then combines the appropriate starting alcohols with the production host in order to produce a fatty ester mixture with the desired fatty ester profile. Thus, the customization of the fatty ester mixture properties can commence prior to the production of any fatty esters.

As will be appreciated by one of skill in the art, in light of the present disclosure, there are numerous optional advantages for some or all of the disclosed embodiments. For example, in some embodiments, the method allows for a reduction in the number of production, concentration, or purification steps. In some embodiments, the disclosed methods can also remove or reduce the need for combining various fatty esters in order to obtain a product with the desired properties. In some embodiments, the disclosed method also allows for a reduction in space and/or an increase in the speed in which a final fatty ester mixture can be created. In some embodiments, the method allows for a single vessel to serve for the fatty ester production process. In some embodiments, mixing and storing vessels can be reduced or eliminated.

The following section presents the meaning of various terms and abbreviations. It also provides various alternative embodiments. Following this section is a general description of various embodiments, which is followed by a section outlining additional specific variations of the various embodiments and parts thereof. This section is then followed by a series of examples that outline various specific embodiments.

### ABBREVIATIONS, TERMS, VARIOUS EMBODIMENTS

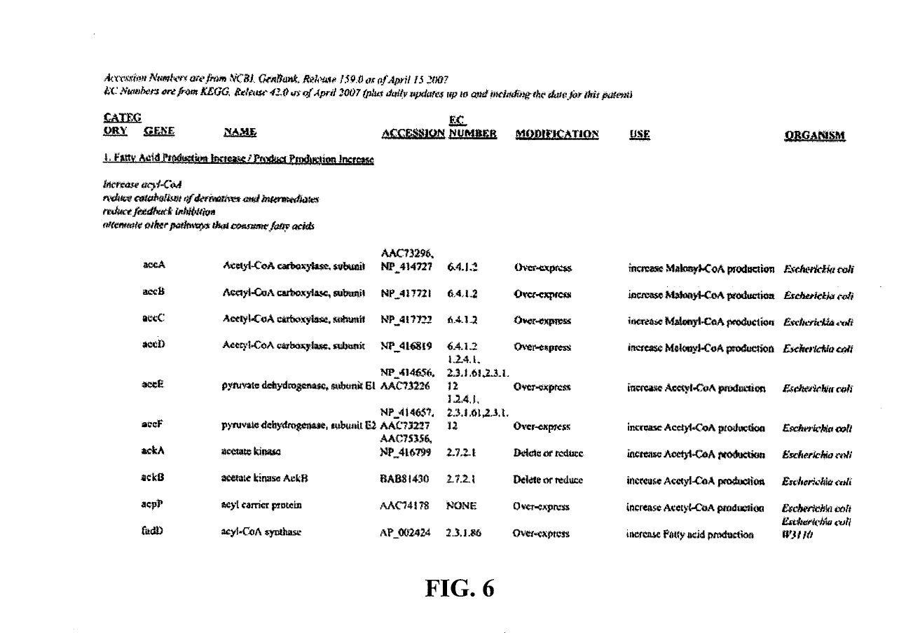

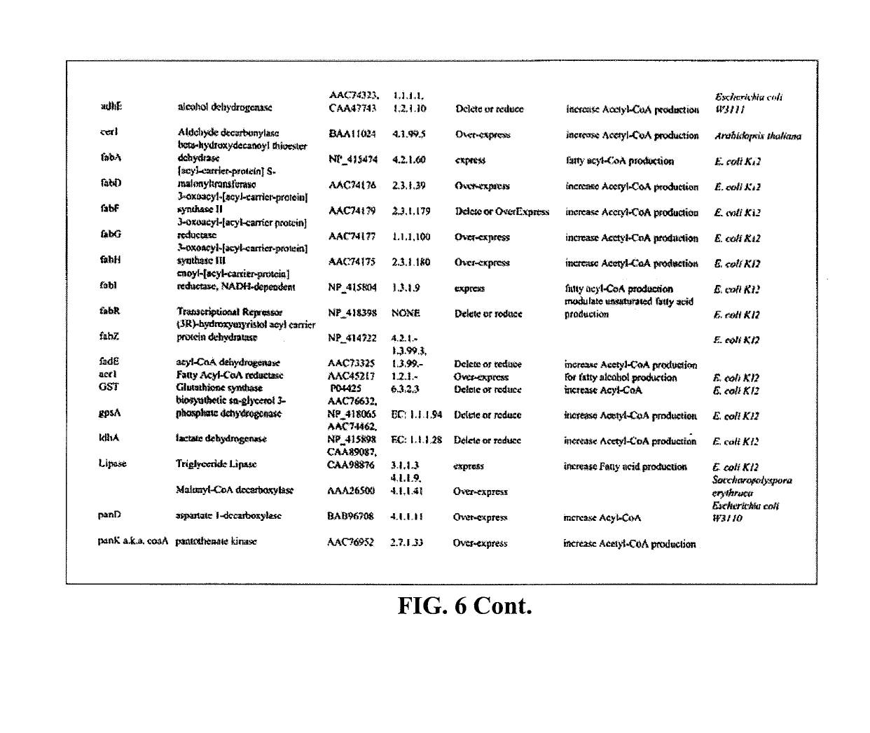

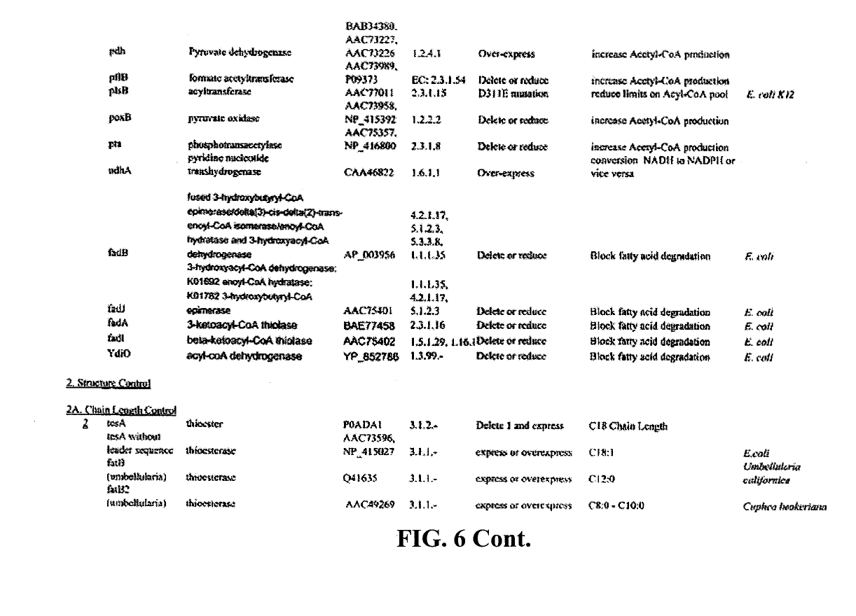

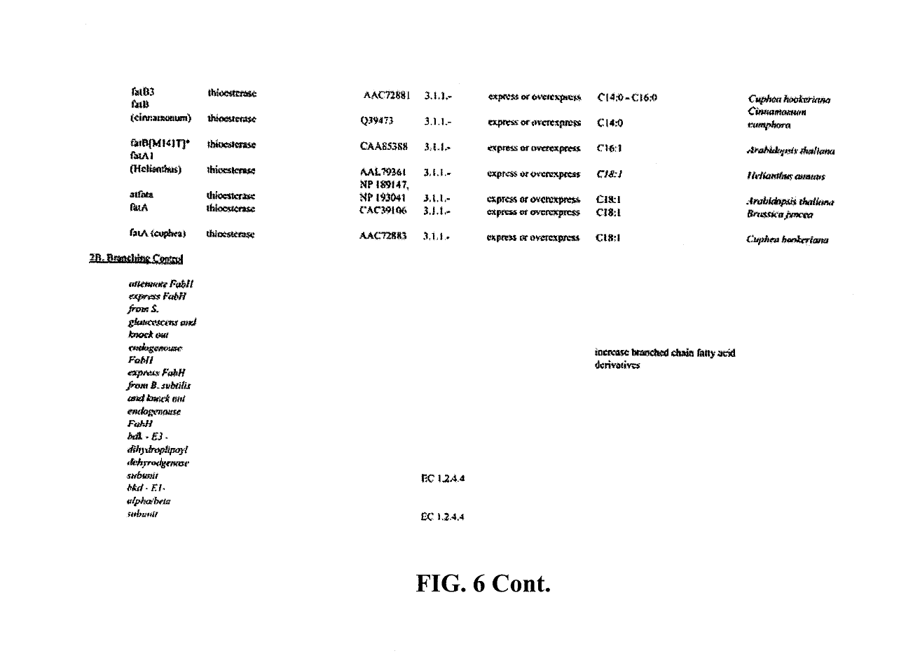

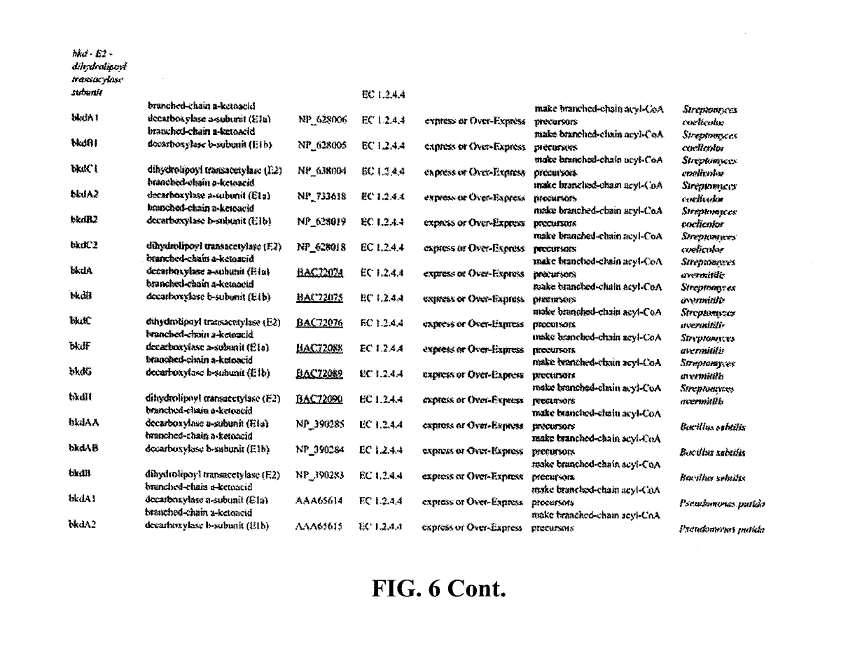

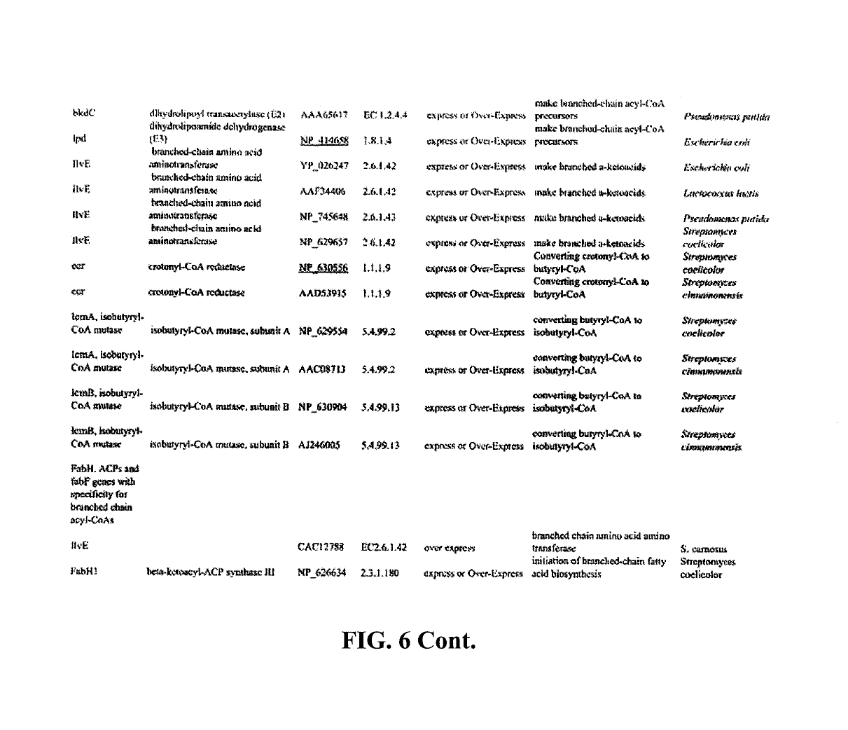

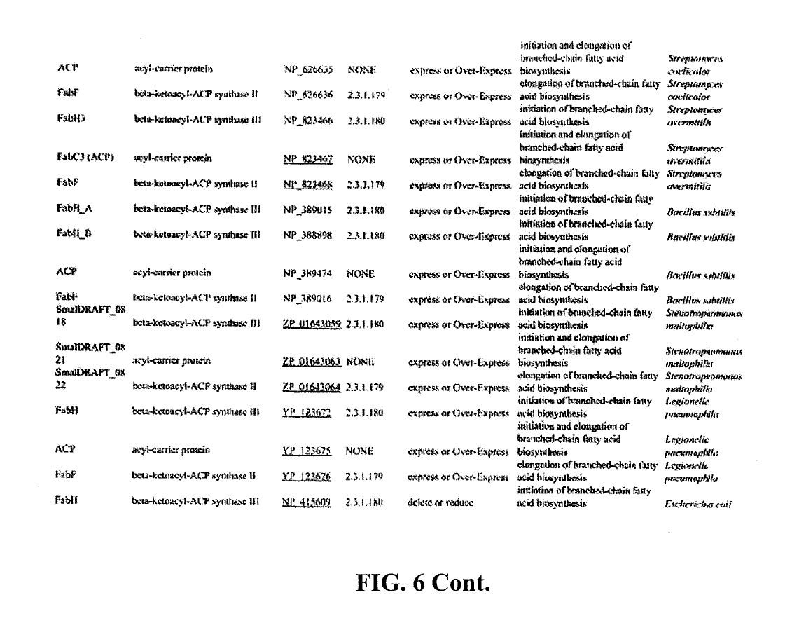

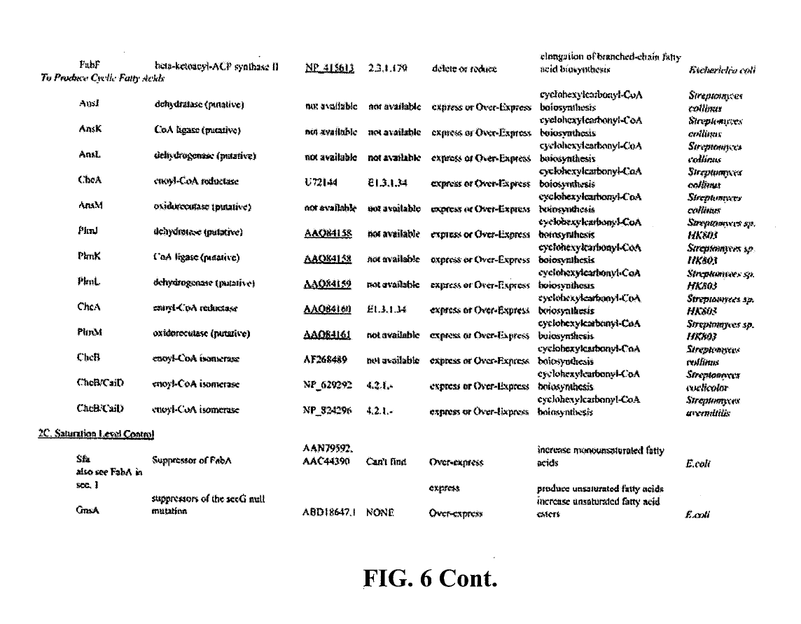

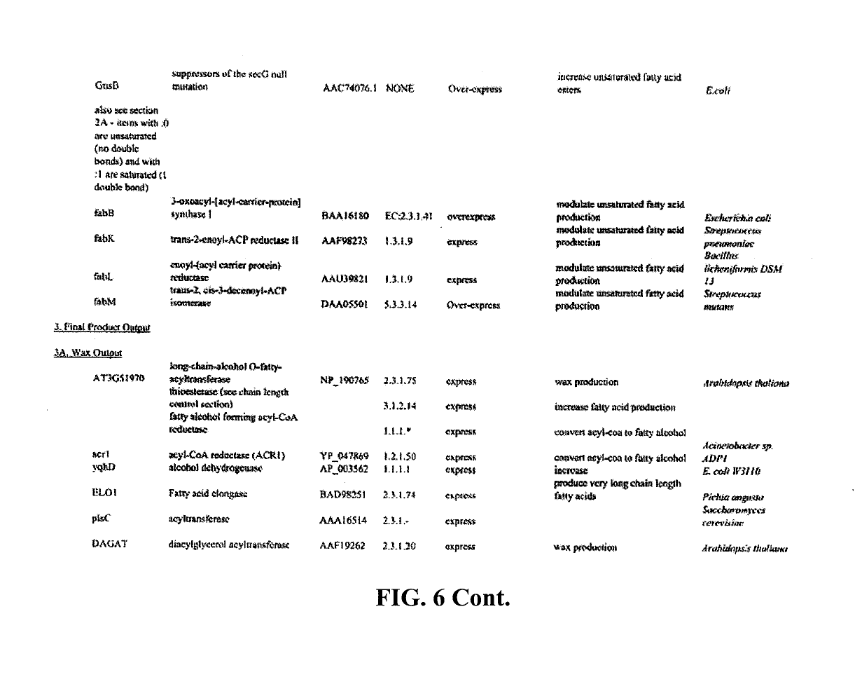

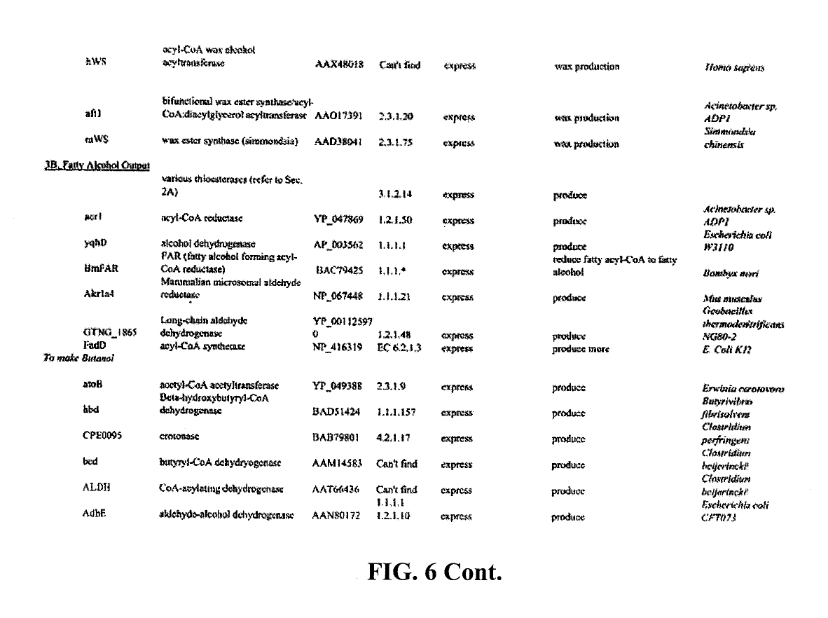

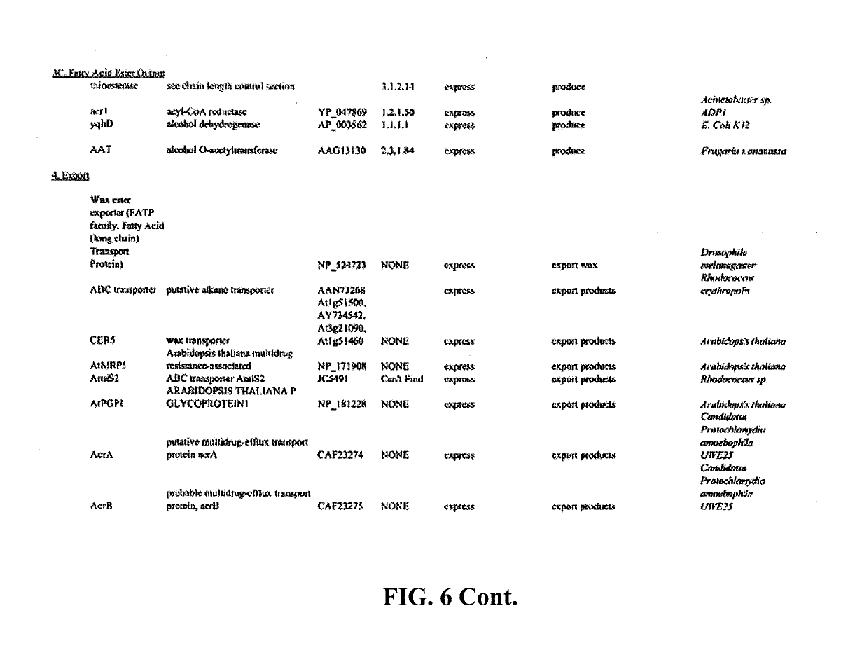

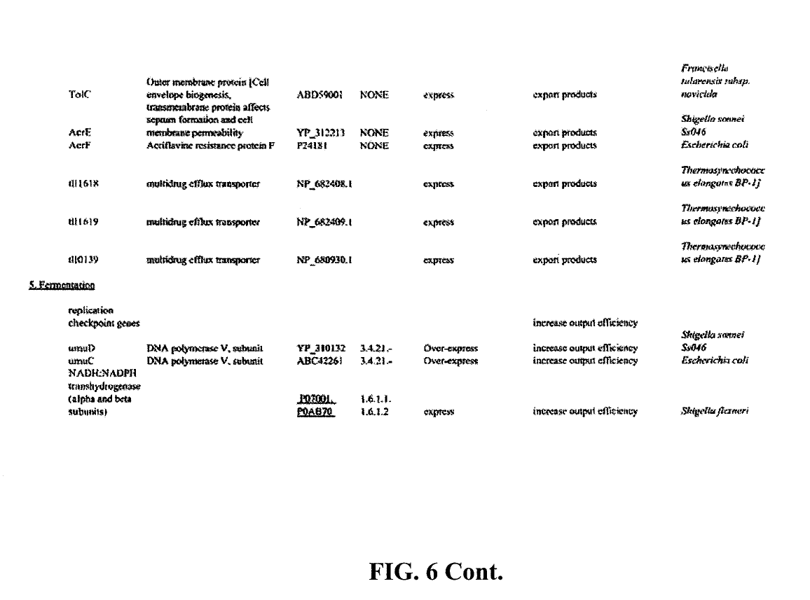

The following explanations of terms and methods are provided to better describe features of the present disclosure and to guide those of ordinary skill in the art in the practice of the present disclosure. As used herein, the singular forms “a,” “an,” or “the” include plural references unless the context clearly dictates otherwise. For example, reference to “a cell” or “the cell” includes one or a plurality of such cells. The term “or” refers to a single element of stated alternative elements or a combination of two or more elements, unless the context clearly indicates otherwise. For example, the phrase “thioesterase activity or fatty alcohol-forming acyl-CoA reductase activity” refers to thioesterase activity, fatty alcohol forming acyl-CoA reductase activity, or a combination of both thioesterase activity and fatty alcohol forming acyl-CoA reductase activity. Additionally, throughout the specification, a reference may be made using an abbreviated gene name or enzyme name, but it is understood that such an abbreviated gene or enzyme name represents the genus of genes or enzymes. For example “fadD” refers to a gene encoding the enzyme “FadD,” as well as genes encoding acyl-CoA synthase (EC 6.2.1.-). Such gene names include all genes encoding the same peptide and homologous enzymes having the same physiological function. Enzyme names include all peptides that catalyze the same fundamental chemical reaction or have the same activity. FIG. 6 provides various abbreviated gene and peptide names, descriptions of their activities, and their enzyme classification numbers. These can be used to identify other members of the class of enzymes having the associated activity and their associated genes, which can be used to produce fatty acid derivatives.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and are not intended to be limiting. Other features of the disclosure are apparent from the following detailed description and the claims.

Accession Numbers: The accession numbers throughout this description are derived from the NCBI database (National Center for Biotechnology Information) maintained by the National Institute of Health, U.S.A. The accession numbers are as provided in the database on Mar. 27, 2007.

Alcohol Composition: Denotes a composition comprising an alcohol molecule and at least one nonalcohol molecule. For example, a mixture comprising ethanol and water would be an alcohol composition. A mixture comprising alcohol and benzene would be another example of an alcohol composition. In some embodiments, at least 0.0001% of the composition is an alcohol (by volume). In some embodiments, such as when alcohol is being produced in the same vessel as the fatty ester, there is no lower requirement for the amount of alcohol that needs to be present in an alcohol composition.

Enzyme Classification Numbers (EC): EC numbers are established by the Nomenclature Committee of the International Union of Biochemistry and Molecular Biology (NC-IUBMB) (available at http://www.chem.qmul.ac.uk/iubmb/enzyme/). The EC numbers provided herein are derived from the KEGG Ligand database, maintained by the Kyoto Encyclopedia of Genes and Genomics, sponsored in part by the University of Tokyo. The EC numbers are as provided in the database on Mar. 27, 2007.

Attenuate: To weaken, reduce or diminish. For example, a polypeptide can be attenuated by modifying the polypeptide to reduce its activity (e.g., by modifying a nucleotide sequence that encodes the polypeptide). In another example, an enzyme that has been modified to be less active can be referred to as attenuated. In some embodiments, a gene or protein that has been removed or deleted can be characterized as having been attenuated.

Biofuel: The term “biofuel” refers to any fuel derived from biomass.

Biomass is a biological material that can be converted into a biofuel. One exemplary source of biomass is plant matter. For example, corn, sugar cane, and switchgrass can be used as biomass. Another non-limiting example of biomass is animal matter, for example cow manure. Biomass also includes waste products from industry, agriculture, forestry, and households. Examples of such waste products which can be used as biomass are fermentation waste, straw, lumber, sewage, garbage and food leftovers. Biomass also includes sources of carbon, such as carbohydrates (e.g., sugars).

In some embodiments, biofuels can be substituted for petroleum based fuels. For example, biofuels are inclusive of transportation fuels (e.g., gasoline, diesel, jet fuel, etc.), heating fuels, and electricity-generating fuels. Biofuels are a renewable energy source. Non-limiting examples of biofuels are biodiesel, hydrocarbons (e.g., alkanes, alkenes, alkynes, or aromatic hydrocarbons), and alcohols derived from biomass.

Biodiesel: Biodiesel is a form of biofuel. Biodiesel can be a substitute of diesel, which is derived from petroleum. Biodiesel can be used in internal combustion diesel engines in either a pure form, which is referred to as “neat” biodiesel, or as a mixture in any concentration with petroleum-based diesel.

Biodiesel can be comprised of hydrocarbons or esters. In some embodiments, biodiesel is comprised of fatty esters, such as fatty acid methyl esters (FAME) or fatty acid ethyl esters (FAEE). In some embodiments, these FAME and FAEE are comprised of fatty acyl moieties having a carbon chain length of about 8-20, 10-18, or 12-16 carbons in length. Fatty esters used as biodiesel may contain carbon chains which are saturated or unsaturated.

Carbon Source: Generally refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, gases (e.g., CO and CO₂), etc. These include, for example, various monosaccharides, such as glucose, fructose, mannose, and galactose; oligosaccharides, such as fructo-oligosaccharide and galacto-oligosaccharide; polysaccharides, such as xylose and arabinose; disaccharides, such as sucrose, maltose, and turanose; cellulosic material, such as methyl cellulose and sodium carboxymethyl cellulose; saturated or unsaturated fatty esters, such as succinate, lactate, and acetate; alcohols, such as ethanol, etc., or mixtures thereof.

The carbon source can additionally be a product of photosynthesis, including, but not limited to glucose. The carbon sounce can additionally be a carbon containing gas, such as carbon dioxide, carbon monoxide, or syngas.

cDNA (complementary DNA): A piece of DNA lacking internal, non-coding segments (introns) and regulatory sequences which determine transcription.

Cloud Point of a Fluid: The temperature at which dissolved solids are no longer completely soluble, precipitating as a second phase giving the fluid a cloudy appearance. This term is relevant to several applications with different consequences.

In the petroleum industry, cloud point refers to the temperature below which wax or other heavy hydrocarbons crystalizes in a crude oil, refined oil or fuel to form a cloudy appearance. The presence of solidified waxes influences the flowing behavior of the fluid, the tendency to clog fuel filters/injectors etc., the accumulation of wax on cold surfaces (e.g., pipeline or heat exchanger fouling), and even the emulsion characteristics with water. Cloud point is an indication of the tendency of the oil to plug filters or small orifices at cold operating temperatures.

The cloud point of a nonionic surfactant or glycol solution is the temperature where the mixture starts to phase separate and two phases appear, thus becoming cloudy. This behavior is characteristic of non-ionic surfactants containing polyoxyethylene chains, which exhibit reverse solubility versus temperature behavior in water and therefore “cloud out” at some point as the temperature is raised. Glycols demonstrating this behavior are known as “cloud-point glycols” and are used as shale inhibitors. The cloud point is affected by salinity, being generally lower in more saline fluids.

Cloud Point Lowering Additive: An additive which may be added to a composition to decrease or lower the cloud point of a solution, as described above.

Combined Fatty Esters, Fatty Ester Mixture, Mixed Fatty Ester Composition, Fatty Ester Composition, or other similar term: Denotes the presence of two or more structurally different fatty esters. In some embodiments, the two or more structurally different fatty esters are present in detectable amounts. In some embodiment, the two or more structurally different fatty esters are present in amounts such that the fatty ester profile of the mixture is different from the fatty ester profile of both of the individual fatty esters. In some embodiments, the two fatty ester differ by their A groups. In some embodiments, the two fatty esters differ by their B groups. In some embodiments, the two fatty esters differ by their A and B groups.

Deletion: The removal of one or more nucleotides from a nucleic acid molecule or one or more amino acids from a protein, the regions on either side being joined together. A deletion can also refer to the missing nucleotide(s) from the nucleic acid molecule.

Desired or Identified Fatty Ester Mixture: is a combination of at least two fatty esters, whose characteristics when combined, will result in (or help result in) a fatty ester mixture with a desired fatty ester profile. The terms can denote an actual composition and/or an ideal mixture that is to be achieved.

Desired Fatty Ester Profile: identifies a specific selection of characteristics that are wanted for a product (which can optionally include, for example, cloud point, cetane number, viscosity, and lubricity). In some embodiments, the desired fatty ester profile also identifies a value for each of the characteristics (e.g., high, low, absent, or a specific or range of values for the characteristic). In some embodiments, the desired fatty ester profile is a construct that is governed by the use or location of use of the fatty ester. In some embodiments, the desired fatty ester profile is used as a guideline for achieving a fatty ester composition with similar properties. While a single fatty ester can have a desired fatty ester profile, in some of the embodiments, the desired fatty ester profile is at least partially achieved through the combination of at least two fatty esters, which when combined will bring the combined fatty esters closer to a desired fatty ester profile.

Detectable: Capable of having an existence or presence ascertained. For example, production of a product from a reactant (e.g., the production of C18 fatty acids) is detectable using the methods provided below.

Endogenous: As used herein, with reference to a nucleic acid molecule and a particular cell or microorganism, “endogenous” refers to a nucleic acid sequence or peptide that is in the cell and was not introduced into the cell using recombinant engineering techniques. For example, a gene that was present in the cell when the cell was originally isolated from nature. A gene is still considered endogenous if the control sequences, such as a promoter or enhancer sequences that activate transcription or translation, have been altered through recombinant techniques.

In some embodiments, if an endogenous sequence is cloned into a different location in the genome of its native cell, or is introduced into the cell as a component of a plasmid, then the gene would no longer be endogenous, but exogenous.

Ester Synthase: An ester synthase is a peptide capable of producing fatty esters. More specifically, an ester synthase is a peptide which converts a thioester to a fatty ester. In a preferred embodiment, the ester synthase converts the thioester, acyl-CoA, to a fatty ester.

In an alternate embodiment, an ester synthase uses a thioester and an alcohol as substrates to produce a fatty ester. Ester synthases are capable of using short and long chain acyl-CoAs as substrates. In addition, ester synthases are capable of using short and long chain alcohols as substrates.

Non-limiting examples of ester synthases are wax synthases, wax-ester synthases, acyl-CoA:alcohol transacylases, acyltransferases, and fatty acyl-coenzyme A:fatty alcohol acyltransferases. Exemplary ester synthases are classified in enzyme classification number EC 2.3.1.75. Exemplary GenBank Accession Numbers are provided in FIG. 6.

Exogenous: As used herein, with reference to a nucleic acid molecule and a particular cell, “exogenous” refers to any nucleic acid molecule that does not originate from that particular cell as found in nature. For example, “exogenous DNA” could refer to a DNA sequence that was inserted within the genomic DNA sequence of a microorganism, or an extra chromosomal nucleic acid sequence that was introduced into the microorganism. Thus, a non-naturally-occurring nucleic acid molecule is considered to be exogenous to a cell once introduced into the cell. A nucleic acid molecule that is naturally-occurring can also be exogenous to a particular cell. For example, an entire coding sequence isolated from an E. coli DH5 alpha cell is an exogenous nucleic acid with respect to a second E. coli DH5 alpha cell once that coding sequence is introduced into the second E. coli DH5 alpha cell, even though both cells are DH5 alpha cells.

Expression: The process by which the inheritable information in a gene, such as the DNA sequence, is made into a functional gene product, such as protein or RNA.

Several steps in the gene expression process may be modulated, including the transcription step, the translational step, and the post-translational modification of the resulting protein. Gene regulation gives the cell control over its structure and function, and it is the basis for cellular differentiation, morphogenesis, and the versatility and adaptability of any organism. Gene regulation may also serve as a substrate for evolutionary change, since control of the timing, location, and amount of gene expression can have a profound effect on the functions (actions) of the gene in the organism.

Expressed genes include genes that are transcribed into messenger RNA (mRNA) and then translated into protein, as well as genes that are transcribed into types of RNA, such as transfer RNA (tRNA), ribosomal RNA (rRNA), and regulatory RNA that are not translated into protein.

Fatty Ester: A fatty ester is an ester. In a preferred embodiment, a fatty ester is any ester made from a fatty acid, for example a fatty acid ester.

In some embodiments, a fatty ester is described as having an A side (i.e., the carbon chain attached to the carboxylate oxygen) and a B side (i.e., the carbon chain comprising the parent carboxylate). In some embodiments, when the fatty ester is derived from the fatty acid biosynthetic pathway, the A side is contributed by an alcohol, and the B side is contributed by a fatty acid.

Any alcohol can be used to form the A side of the fatty esters. For example, the alcohol can be derived from the fatty acid biosynthetic pathway. Alternatively, the alcohol can be produced through non-fatty acid biosynthetic pathways. For example, the alcohol can be produced by the terpenoid pathway or through the branched chain amino acid synthesis or degradation pathways. Moreover, the alcohol can be provided exogenously. For example, the alcohol can be supplied in the production broth in instances where the fatty ester is produced by an organism. Alternatively, a carboxylic acid, such as a fatty acid or acetic acid, can be supplied exogenously in instances where the fatty ester is produced by an organism that can also produce alcohol.

The carbon chains comprising the A side or B side can be of any length. In one embodiment, the A side of the ester is at least about 1, 2, 3, 4, 5, 6, 7, 8, 10, 12, 14, 16, or 18 carbons in length. The B side of the ester is at least about 4, 6, 8, 10, 12, 14, 16, 18, 20, 22, 24, or 26 carbons in length. The A side and/or the B side can be straight or branched chain. The branched chains may have one or more points of branching. In addition, the branched chains can include cyclic branches. Furthermore, the A side and/or B side can be saturated or unsaturated. If unsaturated, the A side and/or B side can have one or more points of unsaturation. As used herein, the B side can include linear alkanes, branched alkanes, and cyclic alkanes (e.g., cycloalkanes).

In some embodiments, the fatty ester is described as follows:

BₙCOOAₙ

Where Bₙ (also known as the B side) is an aliphatic carbon group, such as an alkyl group. In some embodiments, Bₙ comprises, consists, or consists essentially of a chain of carbons at least 5, 6, 7, 8, 9, 10, 11, 12, 13, 14, 15, 16, 17, 18, 19, 20, 21, 22, 23, 24, 25, 26, 27, 28, 29, or 30 carbons in length. Aₙ (also known as the A side) will include at least one carbon and can be an aliphatic group, such as an alkyl group. In some embodiments, the alkyl group comprises, consists or consists essentially of 1, 2, 3, 4, 5, 6, 7, 8, 9, 10, 11, 12, 13, 14, 15, 16, 17, 18, 19, or 20 carbon atoms. A fatty ester mixture may be comprised of fatty esters having a different carbon chain on either the A side, B side, or both the A and B side. The carbon chains may differ with respect to chain length, saturation level, straight chain, branching, etc. Each fatty ester which comprises the fatty ester mixture may impact the overall characteristics and properties of the fatty ester mixture.

In one embodiment, the fatty ester is produced biosynthetically. In this embodiment, first the fatty acid is “activated.” Non-limiting examples of “activated” fatty acids are acyl-CoA, acyl ACP, and acyl phosphate. Acyl-CoA can be a direct product of fatty acid biosynthesis or degradation. In addition, acyl-CoA can be synthesized from a free fatty acid, a CoA, and an adenosine nucleotide triphosphate (ATP). An example of an enzyme which produces acyl-CoA is acyl-CoA synthase

After the fatty acid is activated, it can be readily transferred to a recipient nucleophile. Exemplary nucleophiles are alcohols, thiols, amines, or phosphates.

In another embodiment, the fatty ester can be derived from a fatty acyl-thioester and an alcohol.

In one embodiment, the fatty ester is a wax. The wax can be derived from a long chain alcohol and a long chain fatty acid. In another embodiment, the fatty ester is derived from a long chain alcohol and acetyl-CoA. For example, the long chain alcohol could be derived from fatty acid biosynthesis or from terpenoid biosynthesis. The resulting esters include alkyl acetates, isopentenyl acetate, geranyl acetate, farnesyl acetate, and geranyl acetate. In another embodiment, the fatty ester is a fatty acid thioester, for example fatty acyl Coenzyme A (CoA). In other embodiments, the fatty ester is a fatty acyl panthothenate, an acyl carrier protein (ACP), or a fatty phosphate ester.

Fatty esters have many uses. For examples, fatty esters can be used as a biofuel, a surfactant, or as the intermediate to the synthesis of a commodity, specialty, or fine chemicals, such as fuels, alcohols, olefins, and pharmaceuticals.

Fatty Acid Derivative: The term “fatty acid derivative” includes products made in part from the fatty acid biosynthetic pathway of the production host organism. “Fatty acid derivative” also includes products made in part from acyl-ACP or acyl-ACP derivatives. The fatty acid biosynthetic pathway includes fatty acid synthase enzymes which can be engineered as described herein to produce fatty acid derivatives, and in some examples can be expressed with additional enzymes to produce fatty acid derivatives having desired structural characteristics. Exemplary fatty acid derivatives include, for example, short and long chain alcohols, hydrocarbons, fatty alcohols, and esters, including waxes or fatty esters.

Fatty Acid Derivative Enzymes: All enzymes that may be expressed or overexpressed that affect the production of fatty acid derivatives are collectively referred to herein as fatty acid derivative enzymes. These enzymes may be part of the fatty acid biosynthetic pathway. Non-limiting examples of fatty acid derivative synthases include fatty acid synthases, thioesterases, acyl-CoA synthases, acyl-CoA reductases, alcohol dehydrogenases, alcohol acyltransferases, acetyl-CoA, acetyl transferases, fatty alcohol-forming acyl-CoA reductase, and ester synthases. Fatty acid derivative enzymes convert a substrate into a fatty acid derivative. In some examples, the substrate may be a fatty acid derivative which the fatty acid derivative enzyme converts into a different fatty acid derivative. Additional exemplary fatty acid derivative enzymes include enzymes such as those in glycolysis, acetyl-CoA carboxylase, and panK.

Fatty Ester Characteristic: is a description of the properties of the fatty ester.

Fatty Ester Parameter: is an aspect of the fatty ester molecule itself. Examples of this would include A chain length, B chain length, and degree of satutration.

Fatty Ester Profile: is a description of various characteristics of the fatty ester. In some embodiments, the characteristics relates to the use of the fatty ester as a fuel. Exemplary characteristics include cloud point, cetane number, viscosity, and lubricity.

Fatty Alcohol Forming Peptides: Peptides capable of catalyzing the conversion of acyl-CoA to fatty alcohol, including fatty alcohol forming acyl-CoA reductase (FAR, EC 1.1.1.*), acyl-CoA reductase (EC 1.2.1.50) or alcohol dehydrogenase (EC 1.1.1.1). Additionally, one of ordinary skill in the art will appreciate that some fatty alcohol forming peptides will catalyze other reactions as well. For example, some acyl-CoA reductase peptides will accept other substrates in addition to fatty acyl-CoA. Such non-specific peptides are, therefore, also included. Nucleic acid sequences encoding fatty alcohol forming peptides are known in the art and such peptides are publicly available. Exemplary GenBank Accession Numbers are provided in FIG. 6.

Fraction of Modern Carbon: Fraction of modern carbon (f_{M}) is defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

Fermentation: Fermentation denotes the use of a carbon source by a production host. Fermentation can be aerobic, anaerobic, or variations thereof (such as micro-aerobic).

Functional Deletion: A mutation, partial or complete deletion, insertion, or other variation made to a gene sequence which reduces or inhibits production of the gene product, or renders the gene product non-functional. For example, functional deletion of fabR in E. coli reduces the repression of the fatty acid biosynthetic pathway and allows E. coli to produce more unsaturated fatty acids (uFAs). In some instances a functional deletion is described as a knock-out mutation.

In some embodiments, isolated refers to a naturally-occurring nucleic acid molecule that is not immediately contiguous with both of the sequences with which it is immediately contiguous (one on the 5′ end and one on the 3′ end) in the naturally-occurring genome of the organism from which it is derived.

Heterologous: Heterologous nucleic acid sequence denotes that the nucleic acid sequence has been genetically modified and/or is non-naturally occurring sequence. A sequence can be heterologous, even if the gene has been passed from one organism to another organism. Thus, bacteria produced from an initial bacterium with a heterologous gene would also contain a nucleic acid that is heterologous. Furthermore, differences by deletion or attenuation will also make an altered nucleic acid sequence heterologous.

Isolated: An “isolated” biological component (such as a nucleic acid molecule, protein, or cell) is a biological component that has been substantially separated or purified away from other biological components in which the biological component naturally occurs, such as other chromosomal and extra-chromosomal DNA sequences; chromosomal and extra-chromosomal RNA; and proteins. Nucleic acid molecules and proteins that have been “isolated” include nucleic acid molecules and proteins purified by standard purification methods. The term embraces nucleic acid molecules and proteins prepared by recombinant expression in a production host cell as well as chemically synthesized nucleic acid molecules and proteins.

In one example, isolated refers to a naturally-occurring nucleic acid molecule that is not contiguous with both of the sequences with which it is directly adjacent to (i.e., the sequence on the 5′ end and the sequence on the 3′ end) in the naturally-occurring genome of the organism from which it is derived.

Microorganism: Includes prokaryotic and eukaryotic microbial species from the domains Archaea, Bacteria and Eucarya, the latter including yeast and filamentous fungi, protozoa, algae, or higher Protista. The terms “microbial cells” and “microbes” are used interchangeably with the term microorganism.

Mixed Fatty Ester Fuel or Mixed Fatty Ester Fuel Composition denotes a composition that is useful as a fuel and includes at least two structurally different fatty esters.

Nucleic Acid Molecule: Encompasses both RNA and DNA sequences including, without limitation, cDNA, genomic DNA sequences, and mRNA. The term includes synthetic nucleic acid molecules, such as those that are chemically synthesized or recombinantly produced. The nucleic acid molecule can be double-stranded or single-stranded. When single-stranded, the nucleic acid molecule can be the sense strand or the antisense strand. In addition, a nucleic acid molecule can be circular or linear.

Operably Linked: A first nucleic acid sequence is operably linked to a second nucleic acid sequence when the first nucleic acid sequence is placed in a functional relationship to the second nucleic acid sequence. For instance, a promoter is operably linked to a coding sequence if the promoter is in a position to affect the transcription or expression of the coding sequence. Generally, operably linked DNA sequences are contiguous and may join two protein coding regions, in the same reading frame. Configurations of separate genes which are operably linked and are transcribed in tandem as a single messenger RNA are denoted as operons. Placing genes in close proximity, for example in a plasmid vector, under the transcriptional regulation of a single promoter, constitutes a synthetic operon.

ORF (open reading frame): A series of nucleotide triplets (i.e., codons) coding for amino acids without any termination codons. These sequences are usually translatable into a peptide.

Over-express: When a peptide is present in a greater concentration in a recombinant host cell compared to its concentration in a non-recombinant host cell of the same species. Over-expression can be accomplished using any method known in the art. For example, over-expression can be caused by altering the control sequences in the genomic DNA sequence of a host cell, introducing one or more coding sequences into the genomic DNA sequence, altering one or more genes involved in the regulation of gene expression (e.g., deleting a repressor gene or producing an active activator), amplifying the gene at a chromosomal location (tandem repeats), introducing an extra chromosomal nucleic acid sequence, increasing the stability of the RNA transcribed via introduction of stabilizing sequences, and combinations thereof.

Examples of recombinant microorganisms that over-produce a peptide include microorganisms that express nucleic acid sequences encoding acyl-CoA synthases (EC 6.2.1.-). Other examples include microorganisms that have had exogenous promoter sequences introduced upstream to the endogenous coding sequence of a thioesterase peptide (EC 3.1.2.-). Over-expression also includes elevated rates of translation of a gene compared to the endogenous translation rate for that gene. Methods of testing for over-expression are well known in the art. For example, transcribed RNA levels can be assessed using rtPCR and protein levels can be assessed using SDS page gel analysis.

Partition Coefficient: The partition coefficient, P, is defined as the equilibrium concentration of a compound in an organic phase divided by the concentration at equilibrium in an aqueous phase (e.g., production broth). In one embodiment of the bi-phasic system described herein, the organic phase is formed by the fatty acid derivative during the production process. However, in some examples, an organic phase can be provided, such as by providing a layer of octane, to facilitate product separation. When describing a two phase system, the partition coefficient, P, is usually discussed in terms of log P. A compound with a log P of 1 would partition 10:1 to the organic phase. A compound with a log P of −1 would partition 1:10 to the organic phase. By choosing an appropriate production broth and organic phase, a fatty acid derivative with a high log P value will separate into the organic phase even at very low concentrations in the production vessel.

Process or Production: The term “process” or “production,” when used in reference to a production host denotes the biological manipulation of a production substrate via a production host to result in a product.

Production Broth: Includes any production medium which supports microorganism life (i.e., a microorganism that is actively metabolizing carbon). When noted, a production broth also can refer to “spent” production broth, a production broth which no longer supports microorganism life, and production broths with diminished capacity to support such life, such as being depleted or partially depleted of a carbon source, such as glucose.

Production Host: A production host is a cell that can produce one or more of the products disclosed herein. As disclosed herein, the production host can be modified to express or over-express selected genes, or to have attenuated expression of selected genes. Non-limiting examples of production hosts include plant, animal, human, bacteria, yeast, or filamentous fungi cells. There are various species of production hosts and are generally named by the product they produce. Thus, a fatty ester production host will at least produce fatty esters, an alcohol production host will at least produce an alcohol, and an ethanol production host will at least produce ethanol.

As noted herein, the production hosts can often have heterologous nucleic acid sequences or lack certain otherwise endogenous nucleic acid sequences.

Production Medium: As used herein, can refer to the medium in which a production process occurs. In some embodiments, the production medium can include a production host, a production substrate, and other substances, such as nutrients for the production host, process additives, carriers, or solvents.

Nutrients which can be included in some production media include buffers, minerals, and growth factors. Growth factors can include vitamins, such as biotin, thiamine, pantothenate, nicotinic acid, riboflavin, meso-inositol, folic acid, para-aminobenzoic acid, vitamins A, B (including niacin), C, D, and E, and pyridoxine. Additional growth factors which can be included are peptides or amino acids, such as tryptophan, glutamine, and asparagine. Enzymes can also be included as nutrients or process additives, such as to assist in production, such as by conversion of a substrate to a form more easily fermented by the production host or assisting in the conversion of a substrate to a production product, such as ethanol or a fatty ester.

Minerals which can be included in the production medium include Mg, P, K, Ca, Cu, S, Zn, Fe, Co, Mn, Ni, and Mo and ions, or other inorganic substances, such as ammonium, phosphate, sulfate, chloride, sodium, and borate. Nitrogen sources can also be included in the production media, such as ammonia, urea, ammonium nitrate, ammonium sulfate, grain meal.

Suitable production media are described in Jayme et al., Culture Media for Propagation of Mammalian Cells, Viruses, and Other Biologicals, Advances in Biotechnical Processes 5, p. 1 (1985). Examples of suitable production media include lysogeny broth, corn steep liquor (CSL), M9 minimal medium, SOC medium, Terrific broth, SOB medium, NZM medium, NZCYM medium, MZYM medium, and ZXYT medium.

The chemical and physical properties of the production medium can also be adjusted to suit the needs of a particular production process, production host, or production substrate. For example, in yeast to produce ethanol, the pH of the production medium is typically between about pH 4.0 and about pH 8.8, such as between about pH 4.0 and about pH 5.0. In some examples to produce fatty esters, the pH of the production medium is between about pH 6.0 and about pH 8.0, such as between about pH 6.5 and about pH 7.5 or between about pH 7.0 and about pH 7.4.

In particular examples of yeast fermentation to produce ethanol or when using fatty ester production hosts according to the present disclosure, the temperature of the production medium is maintained at about 10° C. to about 47° C., such as about 30° C. to about 45° C. or about 20° C. to about 40° C. The temperature of the fermentation can be adjusted to produce a desired production rate, for the needs of a particular production host, or can be chosen to facilitate the overall production process. The production temperature can be adjusted during the course of a production, such as being maintained at a higher temperature initially and then decreasing the temperature once production is underway or reaches a certain point, which can be indicated by a chance in the consumption of an input, such as oxygen, or production of an output, such as carbon dioxide. For example, a fatty ester production process can be held at a first temperature for a first part of the production and a second, lower, temperature for a second part of the production, such as after the addition of ethanol to the production.

Production Substrate: Refers to one or more materials which serve as a source of carbon for a production host during a production process (e.g., production of an alcohol or a fatty ester). Different production substrates can be used for different production processes. This will depend on the production host, the production process, and the desired product. For example, when ethanol is the desired product, suitable production substrates include, for example, a carbon source, such as a carbohydrate (e.g., sugar, starch, lignocellulosic biomass, or cellulose), carbon monoxide, or syngas. When fatty esters are the desired product, suitable production substrates include carbon sources, such as, carbohydrates (e.g., glucose), starch, cellulose, lignocellulosic biomass, carbon monoxide, syngas, or ethanol.

Suitable carbohydrate containing substrates for ethanol and fatty ester production include, for example, biological sources, such as sugarcane, sweet sorghum, or sugar beets. Suitable starch sources include, for example, cassava, millet, tapioca, wheat, barley, corn, rice, potatoes, rye, triticale, sorghum grain, sweet potatoes, and Jerusalem artichokes. In further embodiments, the ethanol and fatty esters are produced from biomass, such as grasses (e.g., energy cane, switchgrass, and mycanthus), legumes (e.g., soybeans and peas), algae, seaweed, bagasse, corn stover, pulp and paper mill residues, paper, corn fiber, agricultural residue, plant materials, and wood. In yet further embodiments, the production substrate is a municipal or industrial waste source, such as paper, waste sulfite liquors, or fruit or vegetable wastes from processing plants or canning operations.

In some cases, such as with production substrates having a high content of reducing sugar (e.g., sugar cane and sugar beets), the production substrate can be added to the production medium or production vessel without preprocessing or with minimal processing. For example, a solid production substrate can be broken down into smaller pieces to facilitate production or processing. In particular implementations, the production substrate is milled, either dry or wet, such as using a hammer mill. In further examples, the production substrate is passed through a dispersing machine, such as an in-line machine running the Supramyl process or a batch process using Ultra-Turrax dispersing machines (available from IKA Works, Inc., of Wilmington, N.C.).

However, other production substrates, such as starches or cellulose materials can be subjected to one or more processing steps in order to put the production substrate into a suitable form for production. For example, cellulose materials, such as lingocellulose materials, can be subjected to a hydrolysis, or saccharification, pretreatment step to convert the cellulose to more easily fermentable compounds, such as sugar, including reducing sugars, such as glucose. Hydrolysis, in some implementations, is acid hydrolysis. In other implementations enzymatic hydrolysis is used to convert the cellulose to a more easily fermentable form.

Acid hydrolysis can be carried out using dilute acid, such as 1% sulfuric acid, in a continuous flow reactor at relatively higher temperatures (such as about 215° C.) with a conversion ratio of about 50%. Concentrated acid hydrolysis can be carried out by treating the substrate with 70% sulfuric acid at about 100° F. for 2-6 hours to convert hemicellulose to sugar, followed by treatment with 30 to 40% sulfuric acid for 1 to 4 hours, followed by 70% sulfuric acid treatment for about 1 to about 4 hours. The conversion rate using concentrated acid is typically about 90%. Enzymatic hydrolysis can be carried out using a suitable cellulase enzyme, such as a cellulase derived from Trichoderma viride or Trichoderma reesei. In particular examples, hydrolysis and production are carried out in the same vessel, in a process referred to as Simultaneous Saccharification and Fermentation (SSF).

In particular examples, such as when the production substrate includes a starchy material, the production substrate can be liquefied prior to fermentation, such as by heating and the addition of enzymes, as described in paragraphs 68-71 of U.S. Patent Publication US2007/0082385. The starches can be converted to sugars using various starch reducing enzymes. In particular examples, enzymatic starch reduction is accomplished using as a combination of liquefying α-amylases and saccharifying glucoamylases. Suitable α-amylases include thermostable bacterial α-amylase of Bacillus licheniformis (TBA) (typically used in a production medium having a pH between about 6.2 to about 7.5 at a temperature of about 80° C. to about 85° C.), bacterial alpha-amylase of Bacillus subtilis (BAA) (typically used in a production medium having a pH between about 5.3 to about 6.4 and a temperature of about 50° C.); bacterial alpha-amylase expressed by Bacillus licheniformis (BAB) (typically used in a production medium having a pH between about 4.5 to about 4.8 and a temperature of about 90° C.); and fungal alpha-amylase of Aspergillus oryzae (typically used in a production medium having a pH between about 5.5 to about 8.5 and a temperature between about 35° C. and about 60° C.).

Saccharifying glucoamylases include beta-amylases (such as alpha-1,4-glucan maltohydrolase (EC 3.2.1.2)), and alpha-amylases; glucoamylase (EC 3.2.1.3). Glucoamylase of Aspergillus niger (GAA) (which can operate at a pH range of 3.4 to 5.0 e.g., 4.5 to 5.0; and at a temperature range of 55° C. to 70° C., 60° C.); Glucoamylase of Rhizopus sp. (GAR) (which can operate at a pH range of 4.0 to 6.3, e.g., 4.0 to 5.5; and at a temperature range of 40° C. −60° C. C. Combinations of glucoamylases can also be used, such as GAR and FAA or GAR, GAA, and FAA. Suitable starch reducing enzymes include those present in malted grains.

Grain malting can be accomplished using any suitable technique, many of which are well known in the art. Prior to mashing, a high pressure cooking process, such as in a jet cooker, can be used to release starches from the production substrate. In some examples, mashing is carried out in a stainless steel vessel, which can include a mechanical agitator. The temperature can be maintained at a desired temperature using heaters and cooling coils, such as stainless steel cooling coils. Heat exchangers can be used to conserve energy used in heating and cooling the mash, including spiral-plate, spiral-tubular, plate, or tubular heat exchangers. Suitable mashing processes include cold mashing, the GroBe-Lohmann-Spradau (GLS) process, and milling and mashing process at higher temperatures.

Other souces of a production substrate include carbon containing gases, such carbon monoxide and syngas. Carbon monoxide is a major waste stream from steal mills. When it is compressed it can be fed into a bioreactor as a source of reduced carbon. Syngas is a mixture gases including carbon monoxide, carbon dioxide, and hydrogen that can be generated from carbonaceous materials, such as coal and biomass. There are organisms, such as various Clostridial species, that can use carbon monoxide and/or syngas as a source of carbon and electrons to support growth and as a substrate for chemical production, such as for ethanol and polyhydroxyalkanoate production.

Production System: The various components, including at least a production vessel, used to produce a product, such as an alcohol, a fatty ester, and derivatives thereof, from a production substrate using a production host. The production system can include processes upstream from the production process itself or production vessel, such as substrate handling and conditioning processes. The production system can also include downstream processes, such as processes for separating the product from at least a portion of other components of a mixture from the production vessel. For example, separation can be accomplished by filtration, such as using a membrane filter, a string-discharge filter, or a knife discharge filter. Distillation can also be used to separate the product from at least a portion of the mixture from the production vessel.

In some implementations, the production system includes various components to aid or monitor the process. For example, in some configurations, the system includes defoamers, such as mechanical foam breakers (which, in some examples, are included in the production vessel) or chemical defoamers, such as fatty acids, polyglycols, higher alcohols, or silicones. Particular disclosed production systems include various monitors or sensors, including sensors to measure temperature, pH (such as glass and reference electrodes), dissolved oxygen, foam (such as conductance/capacitance probes), agitation speed (e.g., tachometer), air flow (e.g., rotameter, mass flow meter), pressure, fluid flow, CO₂ content, and specific gravity.

The production system can be run as a batch or continuous process, such as a continuous process with a cell cycle to return a portion of the production host to the production vessel, which can increase product yield. In some embodiments, the process is carried out under vacuum, such as a vacuum fermentation, which includes recycling of at least a portion of the production host. When vacuum fermentation is used, heat from the fermentation process can be used to distill at least a portion of the product, such as ethanol.

Steps can be taken to sterilize the production vessel or other components of the production system. In some methods, heat is used for sterilization, such as treating a surface with pressurized steam for a suitable period of time, for example applying steam at about 120° C. for about 20 minutes. Surfaces can also be disinfected chemically, such as using NaOH, nitric acid, sodium hypochlorite (bleach), ethylene oxide, peracetic acid, ozone, formaldehyde, or antibacterial agents, such as kanamycin, streptomycin, or carbenicillin. In some cases, surfactants are added to the disinfectant in order to help increase disinfectant permeation or penetration. In further implementations, filtration is used to help remove microbes from air or liquid streams. In particular examples, absolute filters having a pore opening of about 0.2 microns are used. In further embodiments, radiation, such as microwave or ultraviolet radiation, can be used to sanitize various system components, including feed or product streams.

Production Vessel: A vessel or container that holds a production host and a substrate, during at least a portion of a production process. Any suitable structure can be used as a production vessel, including those presently in laboratory and commercial use, such as tanks, vats, bags, bottles, flasks, or reactors. In particular implementations, the production vessel can be a stirred tank reactor equipped with a mechanical agitator. Suitable mechanical agitators include paddles, blades, impellers, propellers, or turbines. Tower reactors can also be used as production vessels, particular examples of which are described in U.S. Pat. Nos. 5,888,806 and 4,654,308; and Wieczorek et al., Continuous Ethanol Production by Flocculating Yeast in the Fluidized Bed Bioreactor, FEM Microbio. Rev., 4, pp. 69-74 (1994). In further implementations, the production vessel is a pneumatically agitated reactor, such as tower jet loop, plunging jet, tower jet, and tower pneumatic reactors. In some examples, pneumatic agitation can also serve to increase the oxygen level in the production medium for aerobic production.

In further embodiments, the production vessel is an immobilized microorganism bioreactor. In particular configurations, the production host is immobilized by adsorption onto a preformed carrier (such as wood chips, cellulose, glass, ceramic, or synthetic materials). In some examples, the production host is adsorbed only to the surface of the carrier, while in other examples the production host is also adhered in pores of the carrier. Another method of production host immobilization is by entrapment of the production host in a matrix, such as alginate, kappa-carrageenan, or pectate gels. The production host can also be immobilized by self-aggregation of cells, such as by cross-linking, or by containment of production host behind a barrier, such as encapsulating yeast cells within polyvinyl alcohol beads or plug flow reactors where the production host is retained by one or more support plates.

Various specific implementation of bioreactors using immobilized microorganisms include packed bed reactors, fluidized bed reactors, silicon carbide cartridge loops (silicone carbine rods seeded with yeast cells), or internal loop gas-lift reactors.

In particular embodiments where the production vessel is provided with gas, such as to agitate the vessel contents or to provide an oxygen source for production, the reactor vessel includes a gas inlet, such as a sparger for introducing the gas below the level of the production medium. Suitable gas inlets include one or more nozzles, nozzle clusters, rings or orifices, or porous materials, such as sintered metal or stone. The air source, in some implementations, is supplied by a compressor, such as a rotary, reciprocating, or centrifugal compressor. In some examples, the gas is filtered before introduction into the reactor vessel, such as using a membrane or activated carbon filter.

Promoters and Enhancers: Transcriptional control signals in eukaryotes comprise “promoter” and “enhancer” elements. Promoters and enhancers consist of short arrays of DNA sequences which interact specifically with cellular proteins involved in transcription (Maniatis et al., Science 236:1237, 1987). Promoter and enhancer elements have been isolated from a variety of eukaryotic sources including genes in yeast, insect, mammalian and plant cells. Promoter and enhancer elements can be isolated from viruses.

Analogous control elements, such as promoters and enhancers, are also found in prokaryotes. The selection of a particular promoter and enhancer depends on the cell type used to express the protein of interest. Some eukaryotic and prokaryotic promoters and enhancers have a broad production host cell range while others are functional in a limited subset of production host cells (see, e.g., Voss et al., Trends Biochem. Sci., 11:287, 1986; and Maniatis et al., 1987 supra).

The terms “promoter element,” “promoter,” or “promoter sequence” refer to a DNA sequence that functions as a switch which activates the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Purified: The term “purified” refers to molecules that are removed from their natural environment by, for example, isolation or separation. “Substantially purified” molecules are at least about 60% free, preferably at least about 75% free, and more preferably at least about 90% free from other components with which they are naturally associated. As used herein, the term “purified” or “to purify” also refers to the removal of contaminants from a sample. For example, the removal of contaminants can result in an increase in the percentage of fatty acid derivatives of interest in a sample. For example, after fatty acid derivatives are expressed in plant, bacterial, yeast, or mammalian production host cells, the fatty acid derivatives are purified by the removal of production host cell proteins. After purification, the percentage of fatty acid derivatives in the sample is increased.

The term purified does not require absolute purity; rather, it is intended as a relative term. Thus, for example, a purified fatty acid derivative preparation is one in which the product is more concentrated than the product is in its environment within a cell. For example, a purified fatty ester is one that is substantially separated from cellular components (e.g., nucleic acids, lipids, carbohydrates, and other peptides) that can accompany it. In another example, a purified fatty ester preparation is one in which the fatty ester is substantially free from contaminants, such as those that might be present following production and/or fermentation.

For example, a fatty ester is purified when at least about 50% by weight of a sample is composed of the fatty ester. In another example when at least about 60%, 70%, 80%, 85%, 90%, 92%, 95%, 98%, or 99% or more by weight of a sample is composed of the fatty ester.

Recombinant: A recombinant nucleic acid molecule is one that has a sequence that is not naturally occurring, has a sequence that is made by an artificial combination of two otherwise separated segments of sequence, or both. This artificial combination can be achieved, for example, by chemical synthesis or by the artificial manipulation of isolated segments of nucleic acid molecules, such as genetic engineering techniques. Recombinant is also used to describe nucleic acid molecules that have been artificially manipulated, but contain the same regulatory sequences and coding regions that are found in the organism from which the nucleic acid was isolated. A recombinant protein is a protein derived from a recombinant nucleic acid molecule.

A recombinant or transformed cell is one into which a recombinant nucleic acid molecule has been introduced, such as an acyl-CoA synthase encoding nucleic acid molecule, for example by molecular biology techniques. Transformation encompasses all techniques by which a nucleic acid molecule can be introduced into such a cell, including, but not limited to, transfection with viral vectors, conjugation, transformation with plasmid vectors, and introduction of naked DNA sequence by electroporation, lipofection, and particle gun acceleration.

Release: The movement of a compound out of a cell. The movement can be active or passive. When release is active it can be facilitated by one or more transporter peptides and in some examples it can consume energy. When release is passive, it can be through diffusion through the membrane and can be facilitated by continually collecting the desired compound from the extracellular environment, thus promoting further diffusion. Release of a compound can also be accomplished by lysing a cell.

Surfactants: Substances capable of reducing the surface tension of a liquid in which they are dissolved. They are typically composed of a water-soluble head and a hydrocarbon chain or tail. The water soluble head is hydrophilic and can be either ionic or nonionic. The hydrocarbon chain is hydrophobic. Surfactants are used in a variety of products, including detergents and cleaners, and are also used as auxiliaries for textiles, leather, and paper, in chemical processes, in cosmetics and pharmaceuticals, in the food industry, and in agriculture. In addition, they can be used to aid in the extraction and isolation of crude oils which are found in hard to access environments or in water emulsions.

There are four types of surfactants characterized by varying uses. Anionic surfactants have detergent-like activity and are generally used for cleaning applications. Cationic surfactants contain long chain hydrocarbons and are often used to treat proteins. Amphoteric surfactants contain long chain hydrocarbons and are typically used in shampoos. Non-ionic surfactants are generally used in cleaning products.

Synthase: A synthase is an enzyme which catalyzes a synthesis process. As used herein, the term synthase includes synthases and synthetases.

Transformed or Recombinant Cell: A cell into which a nucleic acid molecule has been introduced. Transformation encompasses all techniques by which a nucleic acid molecule can be introduced into a cell, including, but not limited to, transfection with viral vectors, conjugation, transformation with plasmid vectors, and introduction of naked DNA by electroporation, lipofection, and particle gun acceleration.

Transport Protein: A protein that facilitates the movement of one or more compounds in and/or out of an organism or organelle. In some embodiments, an exogenous DNA sequence encoding an ATP-Binding Cassette (ABC) transport protein will be functionally expressed by the production host so that the production host exports the fatty acid derivative into the culture medium. ABC transport proteins are found in many organisms, such as Caenorhabditis elegans, Arabidopsis thalania, Alcaligenes eutrophus (later renamed Ralstonia eutropha), or Rhodococcus erythropolis. Non-limiting examples of ABC transport proteins include CER5, AtMRP5, AmiS2 and AtPGP1. In a preferred embodiment, the ABC transport protein is CER5 (e.g., AY734542).

In other embodiments, the transport protein is an efflux protein selected from: AcrAB, TolC, or AcrEF from E. coli or tll1618, tll1619, and tll0139 from Thermosynechococcus elongatus BP-1.

In further embodiments, the transport protein is a fatty acid transport protein (FATP) selected from Drosophila melanogaster, Caenorhabditis elegans, Mycobacterium tuberculosis, or Saccharomyces cerevisiae or any one of the mammalian FATPs well known in the art.

Under Conditions that Permit Product Production: Any production conditions that allow a production host to produce a desired product. Exemplary products include acyl-ACP, acyl-CoA and other fatty acid derivatives such as fatty acids, hydrocarbons, fatty alcohols, fatty esters, as well as, in some embodiments, alcohol(s). Production conditions usually comprise many parameters. Exemplary conditions include, but are not limited to, temperature ranges, levels of aeration, and media composition. Each of these conditions, individually and in combination, allows the production host to grow.

Exemplary mediums include liquids or gels. In some embodiments, the medium includes a carbon source, such as glucose, fructose, cellulose, or the like, that can be metabolized by the microorganism directly. In addition, enzymes can be used in the medium to facilitate the mobilization (e.g., the depolymerization of starch or cellulose to fermentable sugars) and subsequent metabolism of the carbon source.

To determine if the culture conditions permit product production, the production host can be cultured for a sufficient time (e.g., about 4, 8, 12, 24, 36, or 48 hours). During culturing or after culturing, samples can be obtained and analyzed to determine if the culture conditions permit product production. For example, the production hosts in the sample or the medium in which the production hosts were grown can be tested for the presence of the desired product. When testing for the presence of a product, assays, such as, but not limited to, TLC, HPLC, GC/FID, GC/MS, LC/MS, MS, as well as those provided in the examples below, can be used.

Vector: A nucleic acid molecule as introduced into a cell, thereby producing a transformed cell. A vector can include nucleic acid sequences that permit it to replicate in the cell, such as an origin of replication. A vector can also include one or more selectable marker genes or other genetic elements known in the art.

Wax: Wax is comprised of fatty esters. In a preferred embodiment, the fatty ester contains an A side and a B side comprised of medium to long carbon chains.

In addition to fatty esters, a wax may comprise other components. For example, wax can also comprise hydrocarbons, sterol esters, aliphatic aldehydes, alcohols, ketones, beta-diketones, triacylglycerols, etc.

### GENERAL EMBODIMENTS

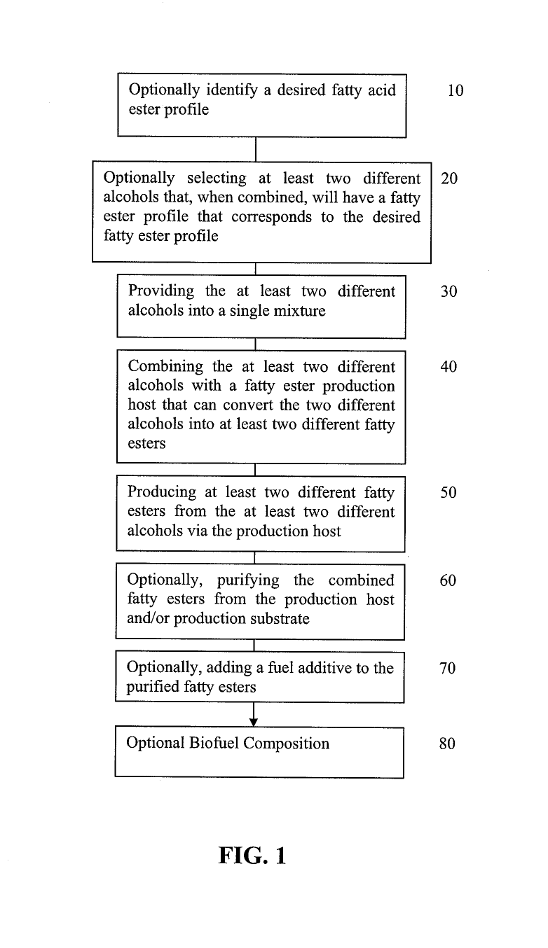

As noted above, by providing a mixture of starting alcohols to a production host, products comprising a mixture of various fatty esters can be created through the production process itself. One embodiment of the invention is disclosed in FIG. 1. As shown in FIG. 1, as an optional initial step, one can identify a desired profile for a final fatty ester mixture 10. This profile can include selected values or ranges of values for a selected combination of characteristics, such as cloud point, cetane number, viscosity, and lubricity. Once the desired value of each relevant characteristics is determined (e.g., a low cloud point and a specific cetane number), the desired set of characteristics can be compared to the profiles of each individual fatty esters in order to determine which individual fatty esters should be combined in order to achieve the desired fatty ester mixture profile. This comparison of the desired mixture profile and the individual profiles of specific lone fatty esters allows one to optionally select at least two different starting alcohols for the production process 20.

As will be appreciated by one of skill in the art, in light of the present disclosure, the starting alcohols are selected so that the production host can convert the mixture of starting alcohols into a desired fatty ester mixture, which can have the desired fatty ester mixture profile. In some embodiments, the alcohols employed in the fatty ester production process will control which A groups are in a fatty ester composition. As shown in the examples below, the specific starting alcohol results in consistent specific esters that vary on their A groups in specific ways. In addition, as described below, the use of specific alcohols also changes the B group in a consistent manner as well. Thus, by selecting a specific combination of starting alcohols, one can manipulate the A groups in the fatty ester mixture.

In some embodiments, following the above optional steps, one then provides at least two starting alcohols 30, and combines the starting alcohols with the fatty ester production host 40 that is then allowed to convert at least some of the alcohols into a fatty ester mixture 50, which will include at least two different fatty esters. One of skill in the art will appreciate that a production substrate will usually be employed in this process and that various parameters can be manipulated so that the production host can more efficiently convert the substrate and alcohols into the fatty ester mixture.

Following this, one can optionally purify the fatty ester mixture to some extent 60. In some embodiments, this purification is sufficient to allow the mixture to be used as a fuel, such as a biofuel such as a biodiesel. In some embodiments, the method can further include adding various fuel additives to the fatty ester mixture (which optionally can be purified) 70. Thus, one can obtain a mixed fatty ester fuel composition, comprising at least two different fatty esters, without having to make or purify the fatty esters separately. In some embodiments, the fatty ester mixture itself is adequate for use as a fuel. In some embodiments, the fatty ester mixture when combined with an additive is ready for use as a fuel. In some embodiments, additional manipulations are performed on the fatty ester mixture. In some embodiments, the fatty ester mixture that results from the above steps can be, or be used as, a biofuel composition 80.

In some embodiments, any one or more of the above steps (10-80) are excluded or repeated. In some embodiments, at least step 50 is performed. In some embodiments, at least steps 40 and 50 are performed. In some embodiments, only steps 40 and 50 are performed. In some embodiments, only step 50 is performed. In some embodiments, the steps are performed in an overlapping manner. In some embodiments the steps are completed before a subsequent step is commenced. In some embodiments, one or more of the above steps are performed at the same time.

Further, specific embodiments of the various aspects described above are provided below.

### Identifying a Desired Fatty Ester Profile.

As noted above, in some embodiments, the method involves identifying a desired fatty ester profile for a fatty ester product (such as a fatty ester mixture). In some embodiments, the fatty ester mixture created by the production host will have this desired profile (of course, in some embodiments, the product from the fatty ester production process can be further manipulated in order to obtain the specific characteristics). As noted above, the desired fatty ester profile includes a specific selection of characteristics that are wanted or should be present in a fatty ester mixture product. As will be appreciated by one of skill in the art, the specific characteristics that are included can vary on a case by case basis. In some embodiments, the first step is to actually select or identify a set of characteristics that a desired mixed fatty ester product will possess. In some embodiments, the characteristics are selected from at least one of the group consisting of: cloud point, cetane number (CN), heat of combustion, exhaust emission (e.g., where appropriate and relative to petrodiesel based fuel), melting point, viscosity (including kinematic viscosity), oxidative stability, and lubricity. In some embodiments, the set of characteristics that are important are selected based upon where, when, and/or how the fatty ester mixture is to be used. In some embodiments, factors such as one or more of: altitude, temperature, agitation, pressure, impurities/additives, type of use (type of engine or motor, mixed with oil, etc.), time of year, federal regulations, state regulations are considered in determining which characteristics of the fatty ester mixture should be enhanced, attenuated, or left alone.

Once the specific characteristics are identified, in some embodiments, one can then further determine the preferred value or range of values for those characteristics. For example, when a low cloud point is important, the cloud point can be less than −20° C. When a high cetane number is important, a higher CN number can be selected (e.g., greater than 30, such as 40 or more).

In some embodiments, the cloud point is low. In some embodiments, the cloud point is less than 0° C., for example −5° C., −10° C., −15° C., −20° C., −25° C., −30° C., −35° C., −40° C., −45° C., −50° C., including any amount lower than any of the preceding values or defined between any two of the preceding values. Generally, the cloud point generally increases with an increase in the number of carbons and/or decreases with an increase in unsaturation.

In some embodiments, the melting point is low. In some embodiments, the melting point is less than 5° C., for example 0° C., −5° C., −10° C., −15° C., −20° C., −25° C., −30° C., −35° C., −40° C., −45° C., −50° C., −55° C., −60° C. including any amount lower than any of the preceding values or defined between any two of the preceding values. In some embodiments, the melting point generally increases with an increase in the number of carbons and decreases with an increase in unsaturation.

In some embodiments, the cetane number is within a specified range. In some embodiments, the cetane number is above 0, for example, 1, 5, 10, 15, 20, 25, 30, 35, 40, 41, 42, 43, 44, 45, 46, 47, 48, 49, 50, 51, 52, 53, 54, 55, 56, 57, 58, 59, 60, 65, 70, 75, 80, 85, or any amount above or below any one of the preceding values or any range defined between any two of the preceding values. Generally, the cetane number increases with an increase in chain length and/or saturation. Generally, branched and/or aromatic compounds have lower cetane numbers.

In some embodiments, the exhaust emissions are relatively low. In some embodiments this is especially true relative to petrodiesel. In some embodiments, the fatty ester, if used as a fuel, will have lower nitrogen oxide, particular matter, hydrocarbons, and or carbon monoxide. In some embodiments, any of these characteristics are used in selecting a desired fatty ester profile and the corresponding fatty ester mixture.

In some embodiments the heat of combustion is within a specified range. In some embodiments, it is no less than 20, 30, 35 or 40 MJ/kg. Generally, the heat of combustion increases with an increase in chain length and/or decreases with an increase in unsaturation.

In some embodiments, the oxidative stability is within a specified range. In some embodiments, an antioxidant is employed to provide additional stability.

In some embodiments, the viscosity is within a specified range. In some embodiments, the kinematic viscosity is within a desired range. Generally, the kinetic viscosity increases with the number of carbon atoms in the fatty ester chain and/or decreases with an increase in unsaturation. In some embodiments, the viscosity is selected to be low. In some embodiments, the viscosity is selected to be high.

In some embodiments, the lubricity is within a specified range. In some embodiments the lubricity is no more than 460 micrometers. In some embodiments, the lubricity is no more than 520 micrometers. In some embodiments, superior lubricity can be obtained through the use of unsaturated esters.

In some embodiments, once one has a desired fatty ester profile, one can then determine a desired fatty ester mixture whose combined characteristics will assist in obtaining the desired fatty ester profile. In some embodiments, this involves selecting the appropriate combination and/or amounts of one or more fatty esters to match various aspects of the desired fatty ester profile. In some embodiments, one can use the characteristics of each individual ester (see, e.g., Tables 1 and 2 below for an exemplary list of various esters and some of their characteristics)

TABLE 1
Characteristics of Fatty Esters Related to Combustion and Emissions

| | | | exhaust emissions | | | |
|---|---|---|---|---|---|---|
| | | heat of | relative to | | | |
| | cetane | combustion | petrodiesel base fuel | | | |
| ester | number | (kJ/mol; kJ/kg) | NO | PM | HC | CO |
| methyl | 39.75 (0.57) | 5523.76/34 907 | | | | |
| octanoate | | | | | | |
| ethyl | 42.19 (0.45) | 6129.56/35 582 | | | | |
| octanoate | | | | | | |
| methyl | 51.63 (0.80) | 6832.24/36 674 | | | | |
| decanoate | | | | | | |
| ethyl | 54.55 (0.95) | 7447.52/37 178 | | | | |
| decanoate | | | | | | |
| methyl | 66.70 (1.49) | 8138.42/37 968 | −5.0 | −83.2 | 13.2 | −28.8 |
| laurate | | | | | | |
| methyl | nd | 9238.27/38 431 | | | | |
| myris- | | | | | | |
| toleate | | | | | | |
| methyl | 85.9 (2.34) | 10 669.20/39 449 | −4.3 | −81.9 | −29.2 | −43.1 |
| palmitate | | | | | | |
| methyl | 56.59 (1.52); | | | | | |
| palmi- | 51.0 (1.21) | | | | | |
| toleate | | | | | | |
| methyl | 101 (3.35) | 11 962.06/40 099 | | | | |
| stearate | | | | | | |
| methyl | 56.55 (1.52); | 11 887.13/40 092 | 6.2 | −72.9 | −54.6 | −49.0 |
| oleate | 59.3 (1.30) | | | | | |
| ethyl | nd | 12 525.17/40 336 | | | | |
| oleate | | | | | | |
| methyl | 37.38 (1.55) | | | | | |
| ricinoleate | | | | | | |
| methyl | 38.2 (0.85) | 11 690.10/39 698 | | | | |
| linoleate | | | | | | |
| methyl | 22.7 | 11 506.00/39 342 | | | | |
| linolenate | | | | | | |

TABLE 2
Melting Points, Kinematic Viscosity, and Oxidative Stability of Fatty Esters

| | | kinematic viscosity (mm²/s) | | | |
|---|---|---|---|---|---|
| ester | mp (° C.) | 40° C. | 0° C. | −10° C. | oxidative stability (h) |
| methyl octanoate | −37.3 (−40) | 1.20 | 2.31 | 3.04 | >24 |
| ethyl octanoate | −44.5 (−43.1) | 1.32 | 2.68 | 3.46 | >24 |
| methyl decanoate | −13.1 (−18) | 1.71 | 4.04 | 4.04 | >24 |
| ethyl decanoate | −19.8 (−20) | 1.87 | 4.28 | 4.28 | >24 |
| methyl laurate | 4.6 (5.2) | 2.43 | solid | | >24 |
| methyl myristoleate | −52.2 | 2.73 | 7.01 | 9.92 | nd |
| ethyl myristoleate | −64.9 | nd | nd | nd | nd |
| methyl palmitate | (30) | 4.38 | solid | | >24 |
| methyl palmitoleate | −33.9 | 3.67 | 10.15 | 14.77 | 2.11 (0.11) |
| ethyl palmitoleate | −36.6 | nd | nd | nd | nd |
| methyl stearate | (39) | 5.85 | solid | | >24 |
| methyl oleate | −19.5 (−19.9) | 4.51 | 14.03 | 21.33 | 2.79 (0.21) |
| ethyl oleate | −20.06 | 4.73 | 14.49 | 22.18 | 2.68 (0.18) |
| methyl ricinoleate | −5.85 | 15.29 | 123.83 | 182.36 | 0.67 (0.02) |
| methyl linoleate | (−35) | 3.65 | 9.84 | 14.10 | 0.94 (0.10) |
| methyl linolenate | (−52) | 3.14 | 7.33 | 10.19 | 0.00 (0.00) |

The above and further characteristics are also discussed in Knothe, “‘Designer’ Biodiesel: Optimizing Fatty Ester Compositions to Improve Fuel Properties” Energy & Fuels 22:1358-1364 (2008), the entirety of which is incorporated by reference. Additional characteristics are provided in The Biodiesel Handbook, by Gerhard Knothe; Jon Harlan and Van Gerpen (Editors), Publisher: Amer Oil Chemists Society (Jan. 30, 2005), the entirety of which is incorporated herein by reference.

One of skill in the art will be able to determine how various amounts of the two or more fatty esters will interact and what the resulting combined fatty ester profile will be for a desired fatty ester mixture. In some embodiments, one of skill in the art can use the method provided in, for example, “Thermodynamic study on cloud point of biodiesel with its fatty acid composition.” Imahara, H., Minami, E., Saka, S., Fuel 85 (2006) 1666-1670, incorporated in its entirety herein.

As will be appreciated by one of skill in the art, the amounts and the actual characteristics of the different fatty esters can be used to both predict a specific characteristic of the fatty ester mixture and/or to determine which fatty esters should be present in a produced fatty ester mixture in order to have the desired properties.

In some embodiments, the first of the at least two different fatty esters has the following formula: B₁COOA₁ and the second of the at least two different fatty esters has the following formula: B₂COOA₂. B₁ is a carbon chain that is at least 6 carbons in length. B₂ is a carbon chain that is at least 6 carbons in length. A₁ is an alkyl group of 1 to 5 carbons in length. A₂ is an alkyl group of 1 to 5 carbons in length. B₁ and B₂ carbon chains have a number of carbon atoms independently selected from the group consisting of: 6, 7, 8, 9, 10, 11, 12, 13, 14, 15, 16, 17, 18, 19, 20, 21, 22, 23, 24, 25, 26, 27, 28, 29, and 30.

### Purifying

In some embodiments, one or more purification procedures can be applied to the fatty ester mixture produced from the production host. In some embodiments, the purification is sufficient to allow the fatty ester mixture to be used as a biofuel, such as biodiesel.

In some embodiments, the amount and/or ratio of one or more of the fatty esters in the mixture is not significantly altered by the purification process. In some embodiments, the amount of one or more of the fatty esters is altered. In some embodiments, the ratio of one fatty ester to another fatty ester is altered during the purification process. As will be appreciated by one of skill in the art, in some embodiments, as long as some amount of at least two fatty esters remains in the fatty ester mixture, the purification step need not take away from the advantages of the customized fuel process described herein.

In some embodiments, all or substantially all of the two or more fatty esters are separated from one another during the purification process. As will be appreciated by one of skill in the art, while these products will no longer be mixed (and thus may contain only a single fatty ester), there can still be advantages to such a process. For example, a single reaction vessel can be used to produce numerous fatty esters. Similarly, a single purification step may be all that is required to separate the fatty esters from the production substrate.

In some embodiments, two or more of the fatty esters are purified from one or more fatty esters produced in the production process. Thus, various subcombinations of fatty esters can be isolated from one or more other fatty esters. In some embodiments, these subcombinations are such that the specific fatty esters within them have similar characteristics (such as cloud point, cetane number, viscosity and/or lubricity). This can allow for a fuel that, while it includes a mixture of fatty esters, has a fatty ester profile that is similar to any one of the fatty esters in isolation. In some embodiments, these subcombinations are such that the specific fatty esters within them have different characteristics (such as cloud point, cetane number, viscosity, lubricity, etc.). In some embodiments, it is this subcombination that possesses the desired fatty ester profile. Thus, in some embodiments, one may remove one or more fatty esters in order to obtain the fatty ester mixture with the desired fatty ester profile.

In some embodiments, converting the alcohols produces a product stream, and the method further comprises performing a separation process to extract the fatty esters from the product stream. In some embodiments, the separation process is chosen from at least one of the group consisting of a filtration, a distillation, and a phase separation process.

In some embodiments, even though the fatty ester mixture comprises two or more fatty esters, both of the fatty esters have the same or similar fatty ester profile. Thus, while in some embodiments two or more fatty esters are produced in combination for a unique fatty ester profile, in other embodiments, two or more fatty esters can be produced together, even though they have the same or similar fatty ester profiles.

### Mixed Fatty Ester Compositions

Compositions that result from at least one of the above outlined processes are also contemplated herein.

In some embodiments, the fatty ester composition comprises a mixture of fatty esters selected from the group consisting of: C12:0, C12:1, C14:0, C14:1, C16:0, C16:1, C18:0, and C18:1. In an alternate embodiment, at least 60% by volume of the fatty esters are C16, C18, or some combination thereof.

In some embodiments, a fatty ester composition comprises a first fatty ester having the following formula: B₁COOA₁ and a second fatty ester has the following formula: B₂COOA₂. B₁ is a carbon chain that is at least 6 carbons in length. B₂ is a carbon chain that is at least 6 carbons in length. A₁ is an alkyl group of 1 to 5 carbons in length. A₂ is an alkyl group of 1 to 5 carbons in length. A₁ is different from A₂. In some embodiments, the ratio of B₁COOA₁ to B₂COOA₂ is about 1:1. In some embodiments, the B₁ and B₂ carbon chains have a number of carbon atoms independently selected from the group consisting of: 6, 7, 8, 9, 10, 11, 12, 13, 14, 15, 16, 17, 18, 19, 20, 21, 22, 23, 24, 25, 26, 27, 28, 29, and 30. In some embodiments, the B₁ carbon chain is polyunsaturated. In some embodiments, the B₂ carbon chain is polyunsaturated. In some embodiments, the B₁ carbon chain is unsaturated. In some embodiments, the B₂ carbon chain is unsaturated. In some embodiments, the B₁ carbon chain is monounsaturated. In some embodiments, the B₂ carbon chain is monounsaturated. In some embodiments, the A₁ group is branched. In some embodiments, the A₁ alkyl group is isopropanol. In some embodiments, the A₂ alkyl group is branched. In some embodiments, the A₂ alkyl group is isopropanol. In some embodiments, the B₁ and/or B₂ group is branched.

In some embodiments, A₁ is different from A₂.

In some embodiments, the composition will include at least two different fatty esters, and can include 3, 4, 5, 6, 7, 8, 9, 10, 11, or more fatty esters. In some embodiments, the number of fatty esters present in the mixture can be from 2 to 100.

In some embodiments, the different fatty esters will differ by at least the number of carbons in the A group of the fatty ester. In some embodiments, the different fatty esters will differ by at least the degree of saturation of the B chain (or will be unsaturated). In some embodiments, the different fatty esters will differ by at least the length of the B chain. In some embodiments, the one or more fatty esters will differ by one or more of the above.

The first and second (and any additional) fatty esters can be present in any amount. In some embodiments, at least one fatty ester is present as at least 0.01% of the resulting fatty ester mixture, for example 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or less than 100 percent, including any amount below any of the preceding values and any number defined between any two of the preceding values. In some embodiments, each fatty acid is present between 0.01% and less than 100 percent of the mixture that includes the at least two fatty esters. Thus, in some embodiments, each fatty ester is 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or less than 100 percent (including any amount below any of the preceding values and any number defined between any two of the preceding values) of the mixture that includes the at least two fatty esters.

In some embodiments, one or more of the fatty esters has a fraction of modern carbon of about 1.003 to about 1.5. In some embodiments, the alkyl group of the A side of one or more of the fatty esters has a number of carbon atoms selected from the group consisting of: 1, 2, 3, 4, and 5. In some embodiments, the B side of one or more of the fatty ester comprises a carbon chain having a number of carbon atoms selected from the group consisting of: 5, 6, 7, 8, 9, 10, 11, 12, 13, 14, 15, 16, 17, 18, 19, 20, 21, 22, 23, 24, 25, 26, 27, 28, 29, and 30. In some embodiments, the number of carbon atoms is selected from the group consisting of 16, 17, and 18. In some embodiments, the fatty ester has a δ¹³C of from about −10.9 to about −15.4.

As noted above, structurally, fatty esters have an A and a B side (or group). In some embodiments, the fatty ester comprises, consists, or consists essentially of the following formula:

BₙCOOAₙ

For convenience of description, “Bₙ” and “Aₙ” are used for generally describing a fatty ester and can apply to one or more of the fatty esters in a mixture. However, unless “B₁” and “A₁” are being used in comparison to “B₂” and “A₂” or some other distinct value, any teaching described herein regarding “B₁” and “A₁” can be applied to a mixture of fatty esters as well.

When discussed in reference to the addition of an alcohol to acyl-CoA, the A side of the fatty ester is used to describe the carbon chain contributed by the alcohol and the B side of the fatty ester is used to describe the carbon chain contributed by the acyl-CoA.

In some embodiments, Aₙ and/or Bₙ are saturated or unsaturated, branched or unbranched, or any combination thereof. In some embodiments, the B side is saturated. In some embodiments, the B side is unsaturated. In some embodiments, Bₙ has a single unsaturated bond. In some embodiments, Bₙ is polyunsaturated. In some embodiments, Aₙ is saturated. In some embodiments, Aₙ is unsaturated. In some embodiments, Aₙ has a single unsaturated bond. In some embodiments, Aₙ is polyunsaturated. In some embodiments, Aₙ and Bₙ can be mono-, di-, or tri-unsaturated simultaneously or independently. In some embodiments, any of the previous Aₙ and Bₙ options can be combined with each other, in any combination.

In some embodiments, the methods described herein permit production of fatty esters of varied length. In some examples, the fatty ester product is a saturated or unsaturated fatty ester product having a carbon atom content limited to between 24 and 46 carbon atoms. In one embodiment, the fatty ester product has a carbon atom content limited to between 24 and 32 carbon atoms. In another embodiment, the fatty ester product has a carbon content of 14 and 20 carbons. In another embodiment, the fatty ester is the methyl ester of C18:1 (or “C_{18:1}” in which “18” denotes the number of carbons present and “1” denotes the number of double bonds). In another embodiment, the fatty ester is the ethyl ester of C16:1. In another embodiment, the fatty ester is the methyl ester of C16:1. In another embodiment, the fatty ester is octadecyl ester of octanol. In another embodiment, the product is a mixture of fatty esters in which greater than about 50%, or greater than about 60%, or greater than about 70%, or greater than about 80%, or greater than about 90% by volume of the component fatty esters have a melting point below about 4 degrees Celsius, below about 0 degrees Celsius, below about −10 degrees Celsius, or below about −20 degrees Celsius.

In some embodiments, Bₙ can have a double bond at one or more points in the carbon chain. Thus, in some embodiments, a 6, 7, 8, 9, 10, 11, 12, 13, 14, 15, 16, 17, 18, 19, 20, 21, 22, 23, 24, 25, 26, 27, 28, 29, or 30 carbon long chain can have 1, 2, 3, 4, 5, 6, 7, 8, 9, 10, 11, 12, 13, 14, 15, 16, 17, 18, 19, 20, 21, 22, 23, or 24 double bonds and 1-24 of those double bonds can be located following carbon 1, 2, 3, 4, 5, 6 7, 8, 9, 10, 11, 12, 13, 14, 15, 16, 17, 18, 19, 20, 21, 22, 23, 24, 25, 26, 27, 28, or 29. In some embodiments, a 1, 2, 3, 4, 5, or 6 carbon chain for Aₙ can have 1, 2, 3, 4, or 5 double bonds and 1-5 of those double bonds can be located following carbon 1, 2, 3, 4, or 5. In some embodiments, any of the above Aₙ groups can be combined with any of the above Bₙ groups.

The production host can be engineered to produce fatty alcohols or short chain alcohols. The production host can also be engineered to produce specific acyl-CoA molecules.

In some embodiments, Bₙ is contributed by a fatty acid produced from de novo synthesis in the host organism. In some embodiments, where the host is additionally engineered to make alcohols, including fatty alcohols, Aₙ is also produced by the host organism. In some embodiments, the Aₙ side can be provided in the medium. As described herein, by selecting the desired thioesterase genes, Bₙ can be designed to have certain carbon chain characteristics. These characteristics include points of unsaturation, branching, and desired carbon chain lengths. For example, at least about 50%, 60%, 70%, 80%, 85%, 90%, 95%, 98%, 99% by volume of the fatty esters produced will have Aₙ and Bₙ that vary by 6, 4, or 2 carbons in length. In some embodiments, Aₙ and Bₙ will also display similar branching and saturation levels. In some embodiments, at least about 50%, 50-60%, 60-70%, 70-80%, 80-90%, 90-95%, 95-98%, 98-99%, or greater percent of the fatty esters produced will have Aₙ and Bₙ that vary by 6, 5, 4, 3, or 2 carbons in length.

Carbon Chain

In some embodiments, the hydrocarbons, fatty alcohols, fatty esters, and waxes disclosed herein are useful as biofuels and specialty chemicals. The products can be produced such that they contain desired branch points, levels of saturation, and carbon chain length. Therefore, these products can be desirable starting materials for use in many applications (FIG. 6 provides a description of the various enzymes that can be used alone or in combination to make various fatty acid derivatives). FIG. 6 also identifies various genes that can be modulated to alter the structure of the fatty acid derivative product. One of ordinary skill in the art will appreciate that some of the genes that are used to alter the structure of the fatty acid derivative can also increase the production of fatty acid derivatives.

Furthermore, biologically produced fatty acid derivatives (including fatty esters) represent a new source of fuels, such as alcohols, biodiesel, diesel and gasoline. Fatty esters and some biofuels made using fatty acid derivatives have not been produced from renewable sources and, as such, are new compositions of matter. These new fatty esters and fuels can be distinguished from fatty esters and fuels derived from petrochemical carbon on the basis of dual carbon-isotopic fingerprinting. Additionally, the specific source of biosourced carbon (e.g. glucose vs. glycerol) can be determined by dual carbon-isotopic fingerprinting (see, U.S. Pat. No. 7,169,588, which is herein incorporated by reference). The following discussion generally outlines two options for distinguishing chemically-identical materials (that have the same structure, but different isotopes). In some embodiments, this apportions carbon in products by the source (and possibly year of growth) of the biospheric (plant) component.

The isotopes, ¹⁴C and ¹³C, provide complementary information to this determination. The radiocarbon dating isotope (¹⁴C), with its nuclear half life of 5730 years, clearly allows one to apportion specimen carbon between fossil (“dead”) and biospheric (“alive”) feedstocks [Currie, L. A. “Source Apportionment of Atmospheric Particles,” Characterization of Environmental Particles, J. Buffle and H. P. van Leeuwen, Eds., 1 of Vol. I of the IUPAC Environmental Analytical Chemistry Series (Lewis Publishers, Inc) (1992) 3 74]. The basic understanding in radiocarbon dating is that the constancy of ¹⁴C concentration in the atmosphere leads to the constancy of ¹⁴C in living organisms. When dealing with an isolated sample, the age of a sample can be deduced approximately by the relationship t=(−5730/0.693)ln(A/A₀) (Equation 1) where t=age, 5730 years is the half-life of radiocarbon, and A and A₀ are the specific ¹⁴C activity of the sample and of the modern standard, respectively [Hsieh, Y., Soil Sci. Soc. Am J., 56, 460, (1992)]. However, because of atmospheric nuclear testing since 1950 and the burning of fossil fuel since 1850, ¹⁴C has acquired a second, geochemical time characteristic. Its concentration in atmospheric CO₂, and hence in the living biosphere, approximately doubled at the peak of nuclear testing, in the mid-1960s. It has since been gradually returning to the steady-state cosmogenic (atmospheric) baseline isotope rate (¹⁴C/¹²C) of ca. 1.2×10¹² with an approximate relaxation “half-life” of 7-10 years. (This latter half-life must not be taken literally; rather, one must use the detailed atmospheric nuclear input/decay function to trace the variation of atmospheric and biospheric ¹⁴C since the onset of the nuclear age.) It is this latter biospheric ¹⁴C time characteristic that holds out the promise of annual dating of recent biospheric carbon. ¹⁴C can be measured by accelerator mass spectrometry (AMS) with results given in units of “fraction of modern carbon” (f_{M}). f_{M} is defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

The stable carbon isotope ratio (¹³C/¹²C) C) provides a complementary route to source discrimination and apportionment. The ¹³C/¹²C ratio in a given biosourced material is a result of the ¹³C/¹²C ratio in atmospheric carbon dioxide at the time the carbon dioxide is fixed and also reflects the precise metabolic pathway. Regional variations also occur. Petroleum, C3 plants (the broadleaf), C₄ plants (the grasses), and marine carbonates all show significant differences in ¹³C/¹²C and their corresponding δ¹³C values. Furthermore, the lipid matter from C3 and C4 plants analyze differently than materials derived from the carbohydrate components of the same plants as a result of the metabolic pathway used in each plant. Within the precision of measurement, ¹³C shows large variations due to isotopic fractionation effects, the most significant of which for the instant invention is the photosynthetic mechanism. The major cause of differences in the carbon isotope ratio in plants is closely associated with differences in the pathway of photosynthetic carbon metabolism in the plants, particularly the reaction occurring during the primary carboxylation (i.e., the initial fixation of atmospheric CO₂). Two large classes of vegetation are those that incorporate the “C3” (or Calvin-Benson) photosynthetic cycle and those that incorporate the “C4” (or Hatch-Slack) photosynthetic cycle. C3 plants, such as hardwoods and conifers, are dominant in the temperate climate zones. In C3 plants, the primary CO₂ fixation or carboxylation reaction involves the enzyme ribulose-1,5-diphosphate carboxylase and the first stable product is a 3-carbon compound. C4 plants, on the other hand, include such plants as tropical grasses, corn and sugar cane. In C4 plants, an additional carboxylation reaction involving another enzyme, phosphoenol-pyruvate carboxylase, is the primary carboxylation reaction. The first stable carbon compound is a 4-carbon acid which is subsequently decarboxylated. The CO₂ thus released is refixed by the C3 cycle.

Both C4 and C3 plants exhibit a range of ¹³C/¹²C isotopic ratios, but typical values are about −10 to −14 per mil (C4) and −21 to −26 per mil (C3) [Weber et al., J. Agric. Food Chem., 45, 2942 (1997)]. Coal and petroleum fall generally in this latter range. The ¹³C measurement scale was originally defined by a zero set by pee dee belemnite (PDB) limestone, where values are given in parts per thousand deviations from this material. The “δ₁₃” values are in parts per thousand (per mil), abbreviated ‰, and are calculated as follows:

$${δ}^{13} C = \frac{{( {}^{13} C / {}^{12} C )}_{sample} - {( {}^{13} C / {}^{12} C )}_{standard}}{{( {}^{13} C / {}^{12} C )}_{standard}} \times 1000$$

Since the PDB reference material (RM) has been exhausted, a series of alternative RMs have been developed in cooperation with the IAEA, USGS, NIST, and other selected international isotope laboratories. Notations for the per mil deviations from PDB is δ¹³. Measurements are made on CO₂ by high precision stable ratio mass spectrometry (IRMS) on molecular ions of masses 44, 45, and 46.

The fatty acid derivatives, fatty esters, and the associated biofuels, chemicals, and mixtures can be distinguished from their petrochemical derived counterparts on the basis of ¹⁴C (fM) and dual carbon-isotopic fingerprinting, indicating new compositions of matter.

In some embodiments, the fatty acid derivatives and fatty esters described herein have utility in the production of biofuels and chemicals. The new fatty acid derivative or fatty ester based product compositions provided herein additionally can be distinguished on the basis of dual carbon-isotopic fingerprinting from those materials derived solely from petrochemical sources. The ability to distinguish these products is beneficial in tracking these materials in commerce. For example, fuels or chemicals comprising both “new” and “old” carbon isotope profiles can be distinguished from fuels and chemicals made only of “old” materials. Hence, the instant materials can be followed in commerce on the basis of their unique profile.

In some examples, a biofuel composition is made that includes a fatty acid derivative or and fatty ester having δ¹³ of from about −10.9 to about −15.4, wherein the fatty acid derivative or fatty ester accounts for at least about 85% by volume of biosourced material (derived from a renewable resource such as cellulosic materials and sugars) in the composition.

In some embodiments, at least one of the fatty esters has a δ¹³ of from about −10.9 to about −15.4. In some embodiments, at least one of the fatty esters has a fraction of modern carbon of about 1.003 to about 1.5. In some embodiments, at least one of the the fatty esters has a δ¹³ of about −28 or greater, for example, a δ¹³ of about −18 or greater, a δ¹³ of about −27 to about −24, or a δ¹³ of about −16 to about −10. In some embodiments, at least one of the fatty esters has a f_{M} ¹⁴C of at least about 1, for example, a f_{M} ¹⁴C of at least about 1.01, a f_{M} ¹⁴C of about 1 to about 1.5, a f_{M} ¹⁴C of about 1.04 to about 1.18, or a f_{M} ¹⁴C of about 1.111 to about 1.124.

In some embodiments, the fatty acid derivative is additionally characterized as having a δ¹³ of from about −10.9 to about −15.4; and the fatty acid derivative accounts for at least about 85% by volume of biosourced material in the composition. In some examples, the fatty acid derivative in the biofuel composition is characterized by having a fraction of modern carbon (f_{M}¹⁴C) of at least about 1.003, 1.010, or 1.5.

In some embodiments, the biofuel composition includes a fatty acid derivative or fatty ester having the formula

X—(CH(R))ₙCH₃

wherein X represents CH₃, —CH₂OR¹; —C(O)OR²; or —C(O)NR³R⁴;

R is, for each n, independently absent, H or lower aliphatic;

n is an integer from 8 to 34, such as from 10 to 24; and

R¹, R², R³ and R⁴ independently are selected from H and lower aliphatic. Typically, when R is lower aliphatic, R represents a branched, unbranched or cyclic lower alkyl or lower alkenyl moiety. Exemplary R groups include, without limitation, methyl, isopropyl, isobutyl, sec-butyl, cyclopentenyl, and the like.

In some embodiments, a biofuel composition is provided that comprises any one of the fatty ester compositions (e.g., mixtures) described herein. In some embodiments, the biofuel is a biodiesel. In some embodiments, the biofuel comprises a fatty ester produced by any of the herein described methods.

As will be appreciated by one of skill in the art, while some of the above methods involve identifying and making a mixed fatty ester composition in light of a desired fatty ester profile, many of the above methods do not require or involve this step. Similarly, the mixed fatty ester compositions themselves need not be the product of a method that involves the identification of a fatty ester profile or a desired fatty ester mixture. Furthermore, in some embodiments, any of the fatty esters described herein can be combined as part of a mixed fatty ester composition.

### Production Conditions

The conditions under which the method can occur can vary based on numerous parameters, such as the size (operational capacity) of the system, the production feeds and hosts used, whether the system is configured for batch or continuous processing, and the desired products. As an example, the following parameters are provided for a fatty ester production process. Of course, these parameters can vary as the process is scaled up or down or different components used.

| | |
|---|---|
| Production vessel Size | 2 L |
| Total initial glucose | 7.5 g in 1.5 L |
| Total glucose added during | 215 g in 0.5 L |
| production | |
| Glucose solution addition rate | 0.1 mL/min ≧ x ≧ 0.5 ml/min |
| Alcohol (such as ethanol) | 45 mL (at start of feed glucose |
| | addition) |
| | 45 mL (after 12 hours) |
| Production host | 100 mg/L |
| pH | 7.2 |
| Temperature | 37° C. (startup) |
| | 30° C. (during glucose/alcohol |
| | addition) |

In some embodiments, the above parameters are scaled up appropriately for 10, 10-100, 100-1000, 10³-10⁴, 10⁴-10⁵, 10⁵-10⁶, 10⁶-10⁷, or more liters.

As will be appreciated by one of skill in the art, the conditions for allowing a production host to process a production substrate into a desired product (e.g., a fatty ester or an alcohol) will vary based upon the specific production host. In some embodiments, the process occurs in an aerobic environment. In some embodiments, the process occurs in an anaerobic environment. In some embodiments, the process occurs in a micro-aerobic environment.

In some embodiments, the amount of production host, production substrate, and alcohol in a fatty ester production process is between about 25 mg/L to about 2 g/L production host, between about 50 g/L and about 200 g/L production substrate, and about 10 mL/L to about 1000 mL/L alcohol, such as between about 75 mL/L and about 250 mg/L production host, about 150 mg/L to about 500 mg/L glucose, and about 25 mL/L to about 100 mL/L ethanol.

In some embodiments, cells (e.g., production hosts) are not added during the production process. In some embodiments, the alcohol composition is added to the fatty ester production host incrementally. In some embodiments, alcohol can be trapped from fatty ester production vessel off gas and be recycled back to the fatty ester production vessel.

### Production Hosts for the Production of Fatty Acid Derivatives and Fatty Esters

As noted above, production hosts are cells that can be used to convert a production substrate into a product, such as a fatty ester. Examples of production hosts include plant, animal, bacteria, yeast, and/or filamentous fungi cells.

In some embodiments, the production hosts comprise heterologous nucleic acid sequences or lack native nucleic acid sequences. In some embodiments, the production host comprises a heterologous nucleic acid sequence encoding a thioesterase (e.g., EC 3.1.2.14). In some embodiments, the production host comprises a heterologous nucleic acid sequence encoding an ester synthase (e.g., EC 2.3.1.75). In some embodiments, the production host comprises a heterologous nucleic acid sequence encoding an acyl-CoA synthase (e.g., E.C.2.3.1.86). In some embodiments, the production host lacks a nucleic acid sequence encoding an acyl-CoA dehydrogenase enzyme. In some embodiments, the production host expresses an attenuated level of an acyl-CoA dehydrogenase enzyme. In some embodiments, any combination of the above is present in a host.

In some embodiments, the production host comprises a heterologous nucleic acid sequence encoding an alcohol acetyltransferase (e.g., EC 2.3.1.84). In some embodiments, the production host comprises a heterologous nucleic acid sequence encoding a fatty alcohol forming acyl-CoA reductase (e.g., EC 1.1.1.*) (wherein “*” denotes that any number applies at this position). In some embodiments, the production host comprises a heterologous nucleic acid sequence encoding an acyl-CoA reductase (e.g., EC 1.2.1.50).

In some embodiments, fatty alcohols having defined carbon chain lengths can be produced by expressing particular exogenous nucleic acid sequences encoding thioesterases (e.g., EC 3.1.2.14) and combinations of acyl-CoA reductases (e.g., EC 1.2.1.50), alcohol dehydrogenases (e.g., EC 1.1.1.1) and fatty alcohol forming acyl-CoA reductases (e.g., EC 1.1.1*). Other enzymes that can be also modulated to increase the production of fatty alcohols include enzymes involved in fatty acid synthesis (e.g., EC 2.3.1.85) and acyl-CoA synthase (e.g., EC 2.3.1.86).

In some embodiments, fatty esters having defined carbon chain lengths can be produced by exogenously expressing particular thioesterases (e.g., EC 3.1.2.14), combinations of acyl-CoA reductase (1.2.1.50), alcohol dehydrogenases (EC 1.1.1.1) and fatty alcohol forming acyl-CoA reductase (e.g., EC 1.1.1*), as well as, acetyl transferase (e.g., EC 2.3.1.84). Other enzymes that can be modulated to increase the production of fatty esters include enzymes involved in fatty acid synthesis (e.g., EC 2.3.1.85) and acyl-CoA synthase (e.g., EC 2.3.1.86).

In some embodiments, the fatty ester production host comprises a recombinant cell. In some embodiments, the recombinant cell lacks a nucleic acid sequence encoding an acyl-CoA dehydrogenase enzyme (E.C. 1.3.99.3, 1.3.99.-) or wherein expression of an acyl-CoA dehydrogenase enzyme is attenuated in the recombinant cell. In some embodiments, the recombinant cell comprises a nucleic acid sequence encoding an ester synthase enzyme. In some embodiments, the recombinant cell comprises a nucleic acid sequence encoding a thioesterase enzyme. In some embodiments, the recombinant cell comprises a nucleic acid sequence encoding an acyl-CoA synthase enzyme.

In some embodiments, the fatty ester production host comprises a heterologous nucleic acid sequence encoding a thioesterase (e.g., EC 3.1.2.14). In some embodiments, the fatty ester production host comprises a heterologous nucleic acid sequence encoding an ester synthase (e.g., EC 2.3.1.75). In some embodiments, the fatty ester production host comprises a heterologous nucleic acid sequence encoding an acyl-CoA synthase (e.g., E.C.2.3.1.86). In some embodiments, the fatty ester production host has attenuated acyl-CoA dehydrogenase activity. In some embodiments, the fatty ester production host lacks an acyl-CoA dehydrogenase gene. In some embodiments, the fatty ester production vessel comprises a fatty ester production host comprising a heterologous nucleic acid sequence encoding an enzyme chosen from the group consisting of: thioesterase (e.g., EC 3.1.2.14), an ester synthase (e.g., EC 2.3.1.75), an alcohol acyltransferase (e.g., EC 2.3.1.84), a fatty alcohol forming acyl-CoA reductase (e.g., EC 1.1.1.*), an acyl-CoA reductase (e.g., EC 1.2.1.50), an alcohol dehydrogenase (e.g., EC 1.1.1.1), and combinations thereof.

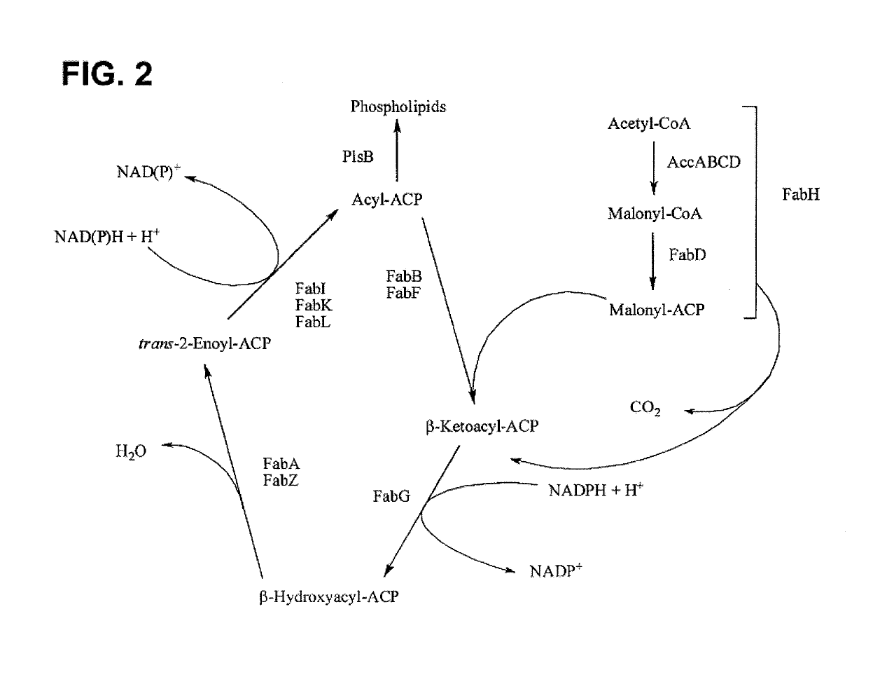

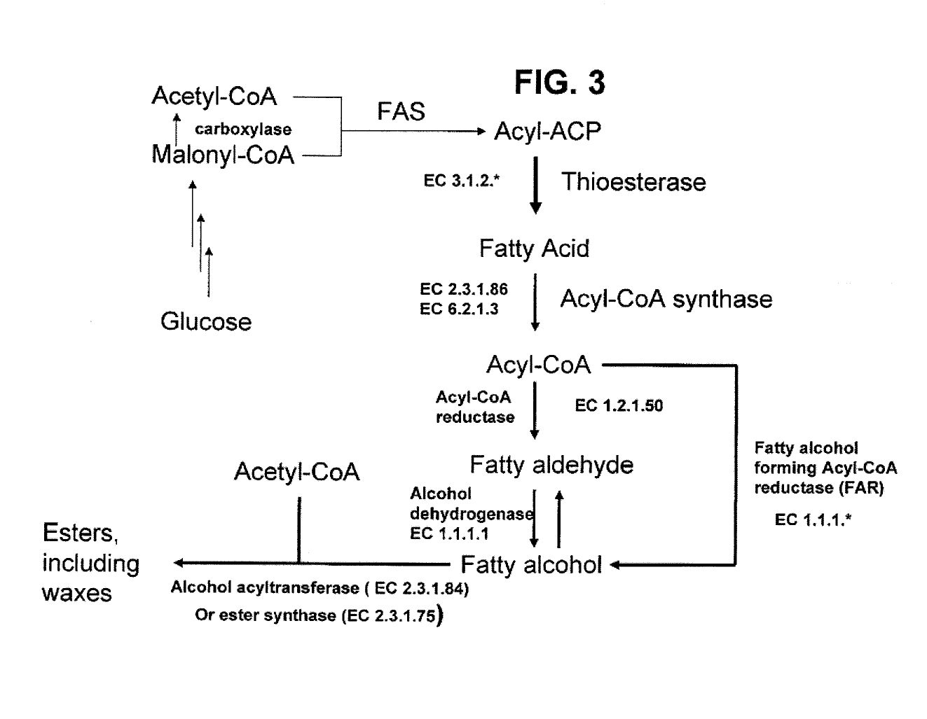

In some embodiments, the host organism that heterologous DNA sequences are transformed into can be a modified host organism, such as an organism that has been modified to increase the production of acyl-ACP or acyl-CoA, reduce the catabolism of fatty acid derivatives and intermediates, or to reduce feedback inhibition at specific points in the biosynthetic pathway. In addition to modifying the genes described herein, additional cellular resources can be diverted to over produce fatty acids. For example, the lactate, succinate and/or acetate pathways can be attenuated or acetyl-CoA carboxylase (ACC) can be over expressed. The modifications to the production host described herein can be through genomic alterations, extrachromosomal expression systems, or combinations thereof. An overview of one such pathway is provided in FIGS. 2 and 3.

A production host, including those for fatty ester production, can include plant, animal, human, bacteria, yeast, or filamentous fungi cells. Additional production hosts include the following: a mammalian cell, plant cell, insect cell, yeast cell, fungus cell, filamentous fungi cell, bacterial cell, a Gram-positive bacteria, a Gram-negative bacteria, the genus Escherichia, the genus Bacillus, the genus Lactobacillus, the genus Rhodococcus, the genus Pseudomonas, the genus Aspergillus, the genus Trichoderma, the genus Neurospora, the genus Fusarium, the genus Humicola, the genus Rhizomucor, the genus Kluyveromyces, the genus Pichia, the genus Mucor, the genus Myceliophtora, the genus Penicillium, the genus Phanerochaete, the genus Pleurotus, the genus Trametes, the genus Chrysosporium, the genus Saccharomyces, the genus Stenotrophamonas, the genus Schizosaccharomyces, the genus Yarrowia, the genus Streptomyces, a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, a Mucor michei cell, a Streptomyces lividans cell, a Streptomyces murinus cell, an Actinomycetes cell, a CHO cell, a COS cell, a VERO cell, a BHK cell, a HeLa cell, a Cv1 cell, an MDCK cell, a 293 cell, a 3T3 cell, a PC12 cell, an E. coli cell, a strain B E. coli cell, a strain C E. coli cell, a strain K E. coli cell, and a strain W E. coli cell. Additional production hosts can be selected from the group consisting of: g-positive bacteria, such as the following: Bacillus (B. lentus, B. brevis, B. stearothermophilus, B. licheniformis, B. alkalophilus, B. coagulans, B. circulans, B pumilis, B. thuringiensis, B. clausii, B. megaterium, B. subtilis, B. amyloliquefaciens), Lactobacillus; g-negative bacteria, such as the following: pseudomonas; Filamentous Fungi, such as the following: Trichoderma (koningii, viride, reesei, longibrachiatum), Aspergillus (awamori, fumigatis, foetidus, nidulans, niger, oryzae), Fusarium, Humicola (Humicola insolens, Humicola lanuginosa), Rhizomucor (R. miehei), Kluyveromyces, Pichia, Mucor (michei), Neurospora, Myceliophtora, Penicillium, Phanerochaete, Pleurotus, Trametes; Yeast, such as the following: Saccharomyces, Schizosaccharomyces, Yarrowia; Actinomycetes, e.g., streptomyces (Streptomyces lividans or Streptomyces murinus); and CHO cells.

In some embodiments, one or more production hosts are present in a production vessel. In some embodiments, one or more production hosts are used to make the same product (e.g., ethanol or fatty esters). In some embodiments, 1, 2, 3, 4, 5, 6, 7, 8, 9, 10, 11, 12, 13, 14, 15, 16, 17, 18, 19, 20 or more types of production hosts are together. In some embodiments, the production host is isolated from other production hosts.

In some embodiments, a production host can be used for alcohol production. In some embodiments, the alcohol produced can include ethanol. For ethanol production, examples of suitable production hosts include yeast, bacteria, Saccharomyces cerevisiae, Saccharomyces distaticus, Saccharomyces uvarum, Schizosaccharomyces pombe, Kluyveromyces marxianus, Kluyveromyces fragilis, Candida pseudotropicalis, Candida brassicae, Clostridium acetobutylicum, Clavispora lusitaniae, Clavispora opuntiae, Pachysolen tannophilus, Bretannomyces clausenii, Zymomonas mobilis, Clostridium thermocellum, and various strains of Escherichia coli, including those described in paragraphs 98-99 of U.S. Patent Publication US2002/0137154 (incorporated herein by reference). Ethanol production hosts also include Klebsiella oxytoca strains, including those described in paragraphs 100-101 of U.S. Patent Publication US2002/0137154 (incorporated herein by reference), as well as the microorganisms described in paragraphs 26-29 of U.S. Patent Publication 2003/0054500 (incorporated herein by reference). Further examples of suitable production hosts for producing ethanol are recombinant bacteria strains, such as B. subtillis, described in U.S. Patent Publication US2005/0158836. Further examples of suitable production hosts for producing ethanol are described in U.S. Pat. No. 7,205,138, which describes methods of producing a product having between 5 to 20% ethanol using a granular starch production substrate, an acid-stable alpha amylase having granular starch hydrolyzing activity, a glucoamylase, and an ethanol producing microorganism, such as yeasts, including strains of Saccharomyces, such as S. cerevisiae. Other suitable production hosts are described in Linden, Industrially Important Strains and Pathways in Handbook of Anaerobic Fermentations, 1988, pp. 59-80; Nakashima, Progress in Ethanol Production With Yeasts, Yeasts, Biotechnology, and Biocatalysis 1990, p 57-84, Benitez et al, Production of Ethanol By Yeast, Handbook of Applied Mycology 4 Fungal Biotechnology 1992, pp. 603-680, and Lida, Fuel Ethanol Production By Immobilized Yeasts and Yeast Immobilization, Industrial Application of Immobilized Biocatalysts, 1993 pp. 163-182 (the entireties of each of which is incorporated by reference).

In some embodiments, alcohols other than ethanol can be produced by one or more alcohol production hosts. As noted herein, in some embodiments, the alcohol production host can produce short chain alcohols, such as ethanol, propanol, isopropanol, isobutanol, and butanol for incorporation in Aₙ using techniques well known in the art. For example, butanol can be made by the host organism. To create butanol producing cells, the E. coli can be further engineered to produce AtoB (acetyl-CoA acetyltransferase) from Escherichia coli K12, β-hydroxybutyryl-CoA dehydrogenase from Butyrivibrio fibrisolvens, crotonase from Clostridium beijerinckii, butyryl CoA dehydrogenase from Clostridium beijerinckii, CoA-acylating aldehyde dehydrogenase (ALDH) from Cladosporium fulvum, and AdhE (aldehyde-alchol dehydrogenase) of Clostridium acetobutylicum in the pBAD24 expression vector under the prpBCDE promoter system. Similarly, ethanol can be produced in a production host using the methods taught by Kalscheuer et al., Microbiology 152:2529-2536, 2006, which is herein incorporated by reference. In some embodiments, a single production host makes both the fatty ester and the alcohol. In some embodiments, two different hosts are responsible for processing the fatty ester and the alcohol.

In some embodiments, a single production host makes both of the fatty esters. In some embodiments, more than one production host is present and different production hosts can make different fatty esters.

Acetyl-CoA—Malonyl-CoA to Acyl-ACP

Fatty acid synthase (FAS) is a group of enzymes that catalyze the initiation and elongation of acyl chains. The acyl carrier protein (ACP) along with the enzymes in the FAS pathway control the length, degree of saturation, and branching of the fatty acids produced. Enzymes that can be included in FAS include AccABCD, FabD, FabH, FabG, FabA, FabZ, FabI, FabK, FabL, FabM, FabB, and FabF. Depending upon the desired product one or more of these genes can be attenuated or over-expressed.

In some embodiments, the fatty acid biosynthetic pathway in the production host uses the precursors acetyl-CoA and malonyl-CoA (FIG. 3). E. coli or other host organisms engineered to overproduce these components can serve as the starting point for subsequent genetic engineering steps to provide the specific output product (such as, fatty esters, hydrocarbons, fatty alcohols). Several different modifications can be made, either in combination or individually, to the host strain to obtain increased acetyl-CoA/malonyl-CoA/fatty acid and fatty acid derivative production. For example, to increase acetyl-CoA production, a plasmid with pdh, panK, aceEF (encoding the E1p dehydrogenase component and the E2p dihydrolipoamide acyltransferase component of the pyruvate and 2-oxoglutarate dehydrogenase complexes), fabH/fabD/fabG/acpP/fabF, and in some examples additional DNA encoding fatty acyl-CoA reductases and aldehyde decarbonylases, all under the control of a constitutive, or otherwise controllable promoter, can be constructed. Exemplary Genbank accession numbers for these genes are: pdh (BAB34380, AAC73227, AAC73226), panK (also known as coaA, AAC76952), aceEF (AAC73227, AAC73226), fabH (AAC74175), fabD (AAC74176), fabG (AAC74177), acpP (AAC74178), fabF (AAC74179).

Additionally, fadE, gpsA, ldhA, pflb, adhE, pta, poxB, ackA, and/or ackB can be knocked-out or their expression levels can be reduced in the engineered microorganism. This can be accomplished by transformation with conditionally replicative or non-replicative plasmids containing null or deletion mutations of the corresponding genes or by substituting promoter or enhancer sequences. Exemplary Genbank accession numbers for these genes are; fadE (AAC73325), gspA (AAC76632), ldhA (AAC74462), pflb (AAC73989), adhE (AAC74323), pta (AAC75357), poxB (AAC73958), ackA (AAC75356), and ackB (BAB81430).

The resulting engineered microorganisms can be grown in a desired environment, for example, one with limited glycerol (e.g., less than 1% w/v in the culture medium). By doing this, these microorganisms will have increased acetyl-CoA production levels. Malonyl-CoA overproduction can be affected by engineering the microorganism, as described above, with DNA encoding accABCD (acetyl-CoA carboxylase, accession number AAC73296, EC 6.4.1.2). Fatty acid overproduction can be achieved by further including DNA encoding lipase (for example, Accessions numbers CAA89087, CAA98876).

In some examples, acetyl-CoA carboxylase (acc) is over-expressed to increase the intracellular concentration thereof by at least 2-fold, such as at least 5-fold, or at least 10-fold relative to native expression levels.

In addition, the plsB (for example, Accession number AAC77011) D311E mutation can be used to remove limitations on the pool of acyl-CoA.

In addition, over-expression of a sfa gene (suppressor of FabAAccession number AAN79592) can be included in the production host to increase production of monounsaturated fatty acids (see, e.g., Rock et al., J. Bacteriology 178:5382-5387, 1996).

Acyl-ACP to Fatty Acid

To engineer a production host for the production of a homogeneous population of fatty acid derivatives, one or more endogenous genes can be attenuated or functionally deleted. In addition, one or more thioesterases can be expressed. For example, C10 fatty acid derivatives can be produced by attenuating thioesterase C18 (for example, accession numbers AAC73596 and POADA1), which uses C18:1-ACP and expressing thioesterase C10 (for example, accession number Q39513), which uses C10-ACP. This results in a relatively homogeneous population of fatty acid derivatives that have a carbon chain length of 10. In another example, C14 fatty acid derivatives can be produced by attenuating endogenous thioesterases that produce non-C14 fatty acids and expressing the thioesterase accession number Q39473 (which uses C14-ACP). In yet another example, C12 fatty acid derivatives can be produced by expressing thioesterases that use C12-ACP (for example, accession number Q41635) and attenuating thioesterases that produce non-C12 fatty acids. acetyl-CoA, malonyl-CoA, and fatty acid overproduction can be verified using methods known in the art, for example, by using radioactive precursors, HPLC, and GC-MS subsequent to cell lysis.

TABLE 3
Thioesterases

| | | | Preferential |
|---|---|---|---|
| Accession | | | product |
| Number | Source Organism | Gene | produced |
| AAC73596 | E. coli | tesA without leader | C18:1 |
| | | sequence | |
| Q41635 | Umbellularia | fatB | C12:0 |
| | california | | |
| Q39513; | Cuphea hookeriana | fatB2 | C8:0-C10:0 |
| AAC49269 | Cuphea hookeriana | fatB3 | C14:0-C16:0 |
| Q39473 | Cinnamonum | fatB | C14:0 |
| | camphorum | | |
| CAA85388 | Arabidopsis thaliana | fatB[M141T]* | C16:1 |
| NP 189147; | Arabidopsis thaliana | fatA | C18:1 |
| NP 193041 | | | |
| CAC39106 | Bradyrhiizobium | fatA | C18:1 |
| | japonicum | | |
| AAC72883 | Cuphea hookeriana | fatA | C18:1 |

*Mayer et al., BMC Plant Biology 7: 1-11, 2007.

Fatty Acid to Acyl-CoA

Production hosts can be engineered using known peptides to produce fatty acids of various lengths. One method of making fatty acids involves increasing the expression of, or expressing more active forms of, one or more acyl-CoA synthases (e.g., EC 2.3.1.86).

As used herein, acyl-CoA synthase includes enzymes in enzyme classification number EC 2.3.1.86, as well as any other enzymes capable of catalyzing the conversion of a fatty acid to an acyl-CoA. Additionally, one of ordinary skill in the art will appreciate that some acyl-CoA synthases will catalyze other reactions as well. For example some acyl-CoA synthases will accept other substrates in addition to fatty acids. Such non-specific acyl-CoA synthase peptides are, therefore, also included. Acyl-CoA synthase sequences are publicly available. Exemplary GenBank Accession Numbers are provided in FIG. 6.

Acyl-CoA to Fatty Alcohol

Production hosts can be engineered using known polypeptides to produce fatty alcohols from acyl-CoA. One method of making fatty alcohols involves increasing the expression of, or expressing more active forms of, fatty alcohol forming acyl-CoA reductases (e.g., EC 1.1.1.*) acyl-CoA reductases (e.g., EC 1.2.1.50), or alcohol dehydrogenases (e.g., EC 1.1.1.1). Hereinafter, fatty alcohol forming acyl-CoA reductases (e.g., EC 1.1.1.*), acyl-CoA reductases (e.g., EC 1.2.1.50), and alcohol dehydrogenases (e.g., EC 1.1.1.1) are collectively referred to as fatty alcohol forming enzymes. In some examples, all three of the fatty alcohol forming genes can be over expressed in a production host. In yet other examples, one or more of the fatty alcohol forming genes can be over-expressed.

As used herein, fatty alcohol forming peptides include peptides in enzyme classification numbers EC 1.1.1.*, 1.2.1.50, and 1.1.1.1, as well as any other peptide capable of catalyzing the conversion of acyl-CoA to fatty alcohol. Additionally, one of ordinary skill in the art will appreciate that some fatty alcohol forming peptides will catalyze other reactions as well. For example, some acyl-CoA reductases will accept other substrates in addition to fatty acids. Such non-specific peptides are, therefore, also included. Fatty alcohol forming peptide sequences are publicly available. Exemplary GenBank Accession Numbers are provided in FIG. 6.

In some embodiments, a microorganism can be engineered to produce fatty alcohols by including a first exogenous DNA sequence encoding a protein capable of converting a fatty acid to a fatty aldehyde and a second exogenous DNA sequence encoding a protein capable of converting a fatty aldehyde to an alcohol. In some examples, the first exogenous DNA sequence encodes a fatty acid reductase. In one embodiment, the second exogenous DNA sequence encodes a mammalian microsomal aldehyde reductase or a long-chain aldehyde dehydrogenase. In a further example, the first and second exogenous DNA sequences are from a multienzyme complex from Arthrobacter AK 19, Rhodotorula glutinins, Acinobacter sp strain M-1, or Candida lipolytica. In one embodiment, the first and second heterologous DNA sequences are from a multienzyme complex from Acinobacter sp strain M-1 or Candida lipolytica.

Additional sources of heterologous DNA sequences encoding enzymes which convert a fatty acid to a long chain alcohol include, but are not limited to, Mortierella alpina (ATCC 32222), Crytococcus curvatus, (also referred to as Apiotricum curvatum), Alcanivorax jadensis (T9T=DSM 12718=ATCC 700854), Acinetobacter sp. HO1-N, (ATCC 14987) and Rhodococcus opacus (PD630 DSMZ 44193).

In one example, the fatty acid derivative is a saturated or unsaturated fatty alcohol having a carbon atom content limited to between 6 and 36 carbon atoms. In another example, the fatty alcohol has a carbon atom content limited to between 24 and 32 carbon atoms.

Appropriate hosts for producing s fatty alcohols can be either eukaryotic or prokaryotic microorganisms. Exemplary hosts include Arthrobacter AK 19, Rhodotorula glutinins, Acinobacter sp strain M-1, Arabidopsis thalania, or Candida lipolytica, Saccharomyces cerevisiae, and E. coli engineered to express acetyl-CoA carboxylase. Hosts which demonstrate an innate ability to synthesize high levels of fatty alcohol precursors in the form of lipids and oils, such as Rhodococcus opacus, Arthrobacter AK 19, Rhodotorula glutinins, E. coli engineered to express acetyl-CoA carboxylase, or other oleaginous bacteria, yeast, and fungi can also be used.

In some embodiments, the expression of exogenous FAS genes originating from different species or engineered variants can be introduced into the host cell to result in the biosynthesis of fatty acid metabolites structurally different (e.g., in length, branching, degree of unsaturation, etc.) than that of the native host. These heterologous gene products can be also chosen or engineered so that they are unaffected by the natural regulatory mechanisms in the host cell and, therefore, function in a manner that is more controllable for the production of the desired commercial product. For example, the FAS enzymes from Bacillus subtilis, Saccharomyces cerevisiae, Streptomyces spp, Ralstonia, Rhodococcus, Corynebacteria, Brevibacteria, Mycobacteria, oleaginous yeast, and the like can be expressed in the production host.

One of ordinary skill in the art will appreciate that when a production host is engineered to produce a fatty acid from the fatty acid biosynthetic pathway that contains a specific level of unsaturation, branching, or carbon chain length, the resulting engineered fatty acid can be used in the production of the fatty acid derivatives. Hence, fatty acid derivatives generated from the production host can display the characteristics of the engineered fatty acid. For example, a production host can be engineered to make branched, short chain fatty acids, and then using the teachings provided herein relating to fatty alcohol production (e.g., including alcohol forming enzymes, such as FAR) the production host produces branched, short chain fatty alcohols. Similarly, a hydrocarbon can be produced by engineering a production host to produce a fatty ester having a defined level of branching, unsaturation, and/or carbon chain length, thus, producing a homogenous hydrocarbon population. Moreover, when an unsaturated alcohol, fatty ester, or hydrocarbon is desired the fatty acid biosynthetic pathway can be engineered to produce low levels of saturated fatty acids and an additional desaturase can be expressed to lessen the saturated product production.

In some embodiments, the fatty ester production host will include an ester synthase. As used herein, ester synthases includes enzymes in enzyme classification number EC 2.3.1.75, as well as any other peptide capable of catalyzing the conversion of an acyl-thioester to fatty esters. Additionally, one of ordinary skill in the art will appreciate that some ester synthases will catalyze other reactions as well. For example, some ester synthases will accept short chain acyl-CoAs and short chain alcohols and produce fatty esters. Such non-specific ester synthases are, therefore, also included. Ester synthase sequences are publicly available. Exemplary GenBank Accession Numbers are provided in FIG. 6. Methods to identify ester synthase activity are provided in U.S. Pat. No. 7,118,896, which is herein incorporated by reference.

In some embodiments, if the desired product is a fatty ester based biofuel, the microorganism is modified so that it produces a fatty ester generated from a renewable energy source. Such a microorganism includes a heterologous DNA sequence encoding an ester synthase that is expressed so as to confer upon said microorganism the ability to synthesize a saturated, unsaturated, or branched fatty ester from a renewable energy source. In some embodiments, the ester synthases include, but are not limited to: fatty acid elongases, acyl-CoA reductases, acyltransferases, ester synthases, fatty acyl transferases, diacylglycerol acyltransferases, acyl-coA wax alcohol acyltransferases, or bifunctional ester synthase/acyl-CoA: diacylglycerol acyltransferases. Bifunctional ester synthase/acyl-CoA: diacylglycerol acyltransferases can be selected from a multienzyme complex from Simmondsia chinensis, Acinetobacter sp. strain ADP1 (formerly Acinetobacter calcoaceticus ADP1), Pseudomonas aeruginosa, Fundibacter jadensis, Arabidopsis thaliana, or Alkaligenes eutrophus. In one embodiment, the fatty acid elongases, acyl-CoA reductases, or ester synthases are from a multienzyme complex from Alkaligenes eutrophus and other organisms known in the literature to produce fatty esters. Additional sources of heterologous DNA encoding ester synthases useful in fatty ester production include, but are not limited to, Mortierella alpina (for example ATCC 32222), Crytococcus curvatus, (also referred to as Apiotricum curvatum), Alcanivorax jadensis (for example T9T=DSM 12718=ATCC 700854), Acinetobacter sp. HO1-N, (for example ATCC 14987), and Rhodococcus opacus (for example PD630, DSMZ 44193).

In some embodiments, useful hosts for producing fatty esters can be either eukaryotic or prokaryotic microorganisms. In some preferred embodiments such hosts include, but are not limited to, Saccharomyces cerevisiae, Candida lipolytica, E. coli Arthrobacter AK 19, Rhodotorula glutinins, Acinobacter sp strain M-1, Candida lipolytica, and other oleaginous microorganisms. Given their high lipid content, fatty acid content, and precursors which can be converted to fatty esters, the preferred hosts are E. coli and Candida lipolytica.

In some embodiments, the ester synthase from Acinetobacter sp. ADP1 (e.g., at locus AAO17391 (described in Kalscheuer and Steinbuchel, J. Biol. Chem. 278:8075-8082, (2003, herein incorporated by reference)) is used. In some embodiments, the ester synthase from Simmondsia chinensis (e,g., at locus AAD38041) is used.

In some embodiments, an ester exporter, such as a member of the FATP family, is used to facilitate the release of fatty esters into the extracellular environment. One example of an ester exporter that can be used is fatty acid (long chain) transport protein CG7400-PA, isoform A from Drosophila melanogaster (e.g., at locus NP_524723).

Genetic Engineering to Increase Fatty Acid Derivative Production

In some embodiments, heterologous DNA sequences involved in biosynthetic pathways for the production of fatty acid derivatives or fatty esters can be introduced stably or transiently into a production host cell using established techniques well known in the art including, for example, electroporation, calcium phosphate precipitation, DEAE-dextran mediated transfection, liposome-mediated transfection, conjugation, transduction, and the like. For stable transformation, a DNA sequence can further include a selectable marker, such as, antibiotic resistance. The selectable marker may provide antibiotic resistance to, for example, neomycin, tetracycline, chloramphenicol, or kanamycin. In addition, genes that complement resistance to auxotrophic deficiencies can be utilized.

In some embodiments, an expression vector that includes a heterologous DNA sequence encoding a protein involved in a metabolic or biosynthetic pathway is provided. Suitable expression vectors include, but are not limited to, viral vectors, such as baculovirus vectors, phage vectors, such as bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli, Pseudomonas pisum and Saccharomyces cerevisiae).

Useful expression vectors can include one or more selectable marker genes to provide a phenotypic trait for selection of transformed host cells. The selectable marker gene encodes a protein necessary for the survival or growth of transformed host cells grown in a selective culture medium. Host cells not transformed with the vector containing the selectable marker gene will not survive in the culture medium. Typical selection genes encode proteins that (a) confer resistance to antibiotics or other toxins (e.g., ampicillin, neomycin, methotrexate, or tetracycline) (b) complement auxotrophic deficiencies, or (c) supply critical nutrients not available from complex media (e.g., the gene encoding D-alanine racemase for Bacilli). In alternative embodiments of this invention, the selectable marker gene is one that encodes dihydrofolate reductase or confers neomycin resistance (for use in eukaryotic cell culture) or one that confers tetracycline or ampicillin resistance (for use in a prokaryotic host cell, such as E. coli).

The biosynthetic pathway gene product-encoding DNA sequence in the expression vector is operably linked to an appropriate expression control sequence, (promoters, enhancers, and the like) to direct synthesis of the encoded gene product. Such promoters can be derived from microbial or viral sources, including CMV and SV40. Depending on the host/vector system utilized, any number of suitable transcription and translation control elements, including constitutive and inducible promoters, transcription enhancer elements, transcription terminators, etc. can be used in the expression vector (see e.g., Bitter et al., Methods in Enzymology, 153:516-544, 1987).

Suitable promoters for use in prokaryotic host cells include, but are not limited to, promoters capable of recognizing the T4, T3, Sp6 and T7 polymerases, the P_{R} and P_{L} promoters of bacteriophage lambda, the trp, recA, heat shock, and lacZ promoters of E. coli, the α-amylase and the α-specific promoters of B. subtilis, the promoters of the bacteriophages of Bacillus, Streptomyces promoters, the int promoter of bacteriophage lambda, the bla promoter of the β-lactamase gene of pBR322, and the CAT promoter of the chloramphenicol acetyl transferase gene. Prokaryotic promoters are reviewed by Glick, J. Ind. Microbiol. 1:277, 1987; Watson et al., MOLECULAR BIOLOGY OF THE GENE, 4th Ed., Benjamin Cummins (1987); and Sambrook et al., supra.

Non-limiting examples of suitable eukaryotic promoters for use within a eukaryotic host are viral in origin and include the promoter of the mouse metallothionein I gene (Hamer et al., J. Mol. Appl. Gen. 1:273, 1982); the TK promoter of Herpes virus (McKnight, Cell 31:355, 1982); the SV40 early promoter (Benoist et al., Nature (London) 290:304, 1981); the Rous sarcoma virus promoter; the cytomegalovirus promoter (Foecking et al., Gene 45:101, 1980); the yeast gal4 gene promoter (Johnston, et al., PNAS (USA) 79:6971, 1982; Silver, et al., PNAS (USA) 81:5951, 1984); and the IgG promoter (Orlandi et al., PNAS (USA) 86:3833, 1989).

The microbial host cell can be genetically modified with a heterologous DNA sequence encoding a biosynthetic pathway gene product that is operably linked to an inducible promoter. Inducible promoters are well known in the art. Suitable inducible promoters include, but are not limited to, promoters that are affected by proteins, metabolites, or chemicals. These include: a bovine leukemia virus promoter, a metallothionein promoter, a dexamethasone-inducible MMTV promoter, a SV40 promoter, a MRP polIII promoter, a tetracycline-inducible CMV promoter (e.g., the human immediate-early CMV promoter) as well as those from the trp and lac operons.

In some examples, a genetically modified host cell is genetically modified with a heterologous DNA sequence encoding a biosynthetic pathway gene product that is operably linked to a constitutive promoter. Suitable constitutive promoters are known in the art and include constitutive adenovirus major late promoter, a constitutive MPSV promoter, and a constitutive CMV promoter.

In some examples a modified host cell is one that is genetically modified with an exongenous DNA sequence encoding a single protein involved in a biosynthesis pathway. In other embodiments, a modified host cell is one that is genetically modified with exongenous DNA sequences encoding two or more proteins involved in a biosynthesis pathway, for example, the first and second enzymes in a biosynthetic pathway.

Where the host cell is genetically modified to express two or more proteins involved in a biosynthetic pathway, those DNA sequences can each be contained in a single or in separate expression vectors. When those DNA sequences are contained in a single expression vector, in some embodiments, the nucleotide sequences will be operably linked to a common control element (e.g., a promoter) which controls expression of all of the biosynthetic pathway protein-encoding DNA sequences in the single expression vector.

When a modified host cell is genetically modified with heterologous DNA sequences encoding two or more proteins involved in a biosynthesis pathway, one of the DNA sequences can be operably linked to an inducible promoter, and one or more of the DNA sequences can be operably linked to a constitutive promoter.

In some embodiments, the intracellular concentration (e.g., the concentration of the intermediate in the genetically modified host cell) of the biosynthetic pathway intermediate can be increased to further boost the yield of the final product. The intracellular concentration of the intermediate can be increased in a number of ways, including, but not limited to, increasing the concentration in the culture medium of a substrate for a biosynthetic pathway; increasing the catalytic activity of an enzyme that is active in the biosynthetic pathway; increasing the intracellular amount of a substrate (e.g., a primary substrate) for an enzyme that is active in the biosynthetic pathway; and the like.

In some examples, the fatty ester, fatty acid derivative, or intermediate is produced in the cytoplasm of the cell. The cytoplasmic concentration can be increased in a number of ways, including, but not limited to, binding of the fatty acid to coenzyme A to form an acyl-CoA thioester. Additionally, the concentration of these acyl-CoAs can be increased by increasing the biosynthesis of acyl-CoA in the cell, such as by over-expressing genes associated with pantothenate biosynthesis (panD) or knocking out the genes associated with glutathione biosynthesis (glutathione synthase).

Branching Including Cyclic Groups

Fatty esters and fatty acid derivatives can be produced that contain branch points, cyclic moieties, and combinations thereof, using the teachings provided herein. In some embodiments, microorganisms that naturally produce straight fatty acids (sFAs) can be engineered to produce branched chain fatty acids (brFAs) by expressing one or more exogenous nucleic acid sequences. For example, E. coli naturally produces straight fatty acids (sFAs). To engineer E. coli to produce brFAs, several genes can be introduced and expressed that provide branched precursors (bkd operon) and allow initiation of fatty acid biosynthesis from branched precursors (fabH). Additionally, the organism can express genes for the elongation of brFAs (e.g. ACP, fabF) and/or deleting the corresponding E. coli genes that normally lead to sFAs and would compete with the introduced genes (e.g. FabH, FabF).

The branched acyl-CoAs 2-methyl-buturyl-CoA, isovaleryl-CoA and isobuturyl-CoA are the precursors of brFA. In most brFA-containing microorganisms, they are synthesized in two steps (described in detail below) from branched amino acids (isoleucine, leucine and valine) (Kadena, Microbiol. Rev. 55: pp. 288, 1991). To engineer a microorganism to produce brFAs, or to overproduce brFAs, expression or over-expression of one or more of the enzymes in these two steps can be engineered. For example, in some instances the production host can have an endogenous enzyme that can accomplish one step and, therefore, only enzymes involved in the second step need to be expressed recombinantly.

The first step in forming branched fatty acids is the production of the corresponding α-keto acids by a branched-chain amino acid aminotransferase. E. coli has such an enzyme, IlvE (EC 2.6.1.42; Genbank accession YP_026247). In some examples, a heterologous branched-chain amino acid aminotransferase may not be expressed. However, E. coli IlvE or any other branched-chain amino acid aminotransferase (e.g. ilvE from Lactococcus lactis (Genbank accession AAF34406), ilvE from Pseudomonas putida (Genbank accession NP_745648) or ilvE from Streptomyces coelicolor (Genbank accession NP_629657)) can be over-expressed in a host microorganism if the aminotransferase reaction turns out to be rate limiting in brFA biosynthesis in the host organism chosen for fatty acid derivative production.

The second step, the oxidative decarboxylation of the α-ketoacids to the corresponding branched-chain acyl-CoA, is catalyzed by a branched-chain α-keto acid dehydrogenase complexes (bkd; EC 1.2.4.4.) (Denoya et al. J. Bacteriol. 177: pp. 3504, 1995), which consists of E1α/β (decarboxylase), E2 (dihydrolipoyl transacylase), and E3 (dihydrolipoyl dehydrogenase) subunits and are similar to pyruvate and α-ketoglutarate dehydrogenase complexes. Table 4 shows potential bkd genes from several microorganisms that can be expressed in a production host to provide branched-chain acyl-CoA precursors. Basically, every microorganism that possesses brFAs and/or grows on branched-chain amino acids can be used as a source to isolate bkd genes for expression in production hosts, for example, E. coli. Furthermore, E. coli has the E3 component (as part of its pyruvate dehydrogenase complex; lpd, EC 1.8.1.4, Genbank accession NP_414658). It can, therefore, only express the E1α/β and E2 bkd genes.

TABLE 4
Bkd genes from selected microorganisms

| Organism | Gene | Genbank Accession # |
|---|---|---|
| Streptomyces coelicolor | bkdA1 (E1α) | NP_628006 |
| | bkdB1 (E1β) | NP_628005 |
| | bkdC1 (E2) | NP_638004 |
| Streptomyces coelicolor | bkdA2 (E1α) | NP_733618 |
| | bkdB2 (E1β) | NP_628019 |
| | bkdC2 (E2) | NP_628018 |
| Streptomyces avermitilis | bkdA (E1a) | BAC72074 |
| | bkdB (E1b) | BAC72075 |
| | bkdC (E2) | BAC72076 |
| Streptomyces avermitilis | bkdF (E1α) | BAC72088 |
| | bkdG (E1β) | BAC72089 |
| | bkdH (E2) | BAC72090 |
| Bacillus subtilis | bkdAA (E1α) | NP_390288 |
| | bkdAB (E1β) | NP_390288 |
| | bkdB (E2) | NP_390288 |
| Pseudomonas putida | bkdA1 (E1α) | AAA65614 |
| | bkdA2 (E1β) | AAA65615 |
| | bkdC (E2) | AAA65617 |

In another example, isobuturyl-CoA can be made in a production host, for example, in E. coli through the coexpression of a crotonyl-CoA reductase (e.g., EC 1.1.1.9) and isobuturyl-CoA mutase (large subunit IcmA, EC 5.4.99.2; small subunit IcmB, EC 5.4.99.13) (Han and Reynolds J. Bacteriol. 179: pp. 5157, 1997). Crotonyl-CoA is an intermediate in fatty acid biosynthesis in E. coli and other microorganisms. Examples for ccr and icm genes from selected microorganisms are given in Table 5.

TABLE 5
Ccr and icm genes from selected microorganisms

| Organism | Gene | Genbank Accession # |
|---|---|---|
| Streptomyces coelicolor | ccr | NP_630556 |
| | icmA | NP_629554 |
| | icmB | NP_630904 |
| Streptomyces cinnamonensis | ccr | AAD53915 |
| | icmA | AAC08713 |
| | icmB | AJ246005 |

In addition to expression of the bkd genes (see above), the initiation of brFA biosynthesis utilizes β-ketoacyl-acyl-carrier-protein synthase III (FabH, EC 2.3.1.41) with specificity for branched chain acyl-CoAs (Li et al. J. Bacteriol. 187: pp. 3795, 2005). Examples of such FabHs are listed in Table 6. fabH genes that are involved in fatty acid biosynthesis of any brFA-containing microorganism can be expressed in a production host. The Bkd and FabH enzymes from production hosts that do not naturally make brFA may not support brFA production and, therefore, bkd and fabH can be expressed recombinantly. Similarly, the endogenous level of Bkd and FabH production may not be sufficient to produce brFA. Therefore, they can be over-expressed. Additionally, other components of fatty acid biosynthesis machinery can be expressed, such as acyl carrier proteins (ACPs) and β-ketoacyl-acyl-carrier-protein synthase II (fabF, EC 2.3.1.41) (candidates are listed in Table 6). In addition to expressing these genes, some genes in the endogenous fatty acid biosynthesis pathway can be attenuated in the production host. For example, in E. coli the most likely candidates to interfere with brFA biosynthesis are fabH (Genbank accession # NP_415609) and/or fabF genes (Genbank accession # NP_415613).

As mentioned above, through the combination of expressing genes that support brFA synthesis and alcohol synthesis branched chain alcohols can be produced. For example, when an alcohol reductase, such as Acr1 from Acinetobacter baylyi ADP1 is coexpressed with a bkd operon, E. coli can synthesize isopentanol, isobutanol, or 2-methyl butanol. Similarly, when Acr1 is coexpressed with ccr/icm genes, E. coli can synthesize isobutanol.

In order to convert a production host, such as E. coli, into an organism capable of synthesizing co-cyclic fatty acids (cyFAs), several genes can be introduced and expressed that provide the cyclic precursor cyclohexylcarbonyl-CoA (Cropp et al. Nature Biotech. 18: pp. 980, 2000). One or more of the genes listed in Table 6 (e.g., fabH, ACP, and fabF) can be expressed to allow initiation and elongation of co-cyclic fatty acids. Alternatively, the homologous genes can be isolated from microorganisms that make cyFAs and expressed in E. coli.

TABLE 6
fabH, ACP and fabF genes from selected microorganisms with brFAs

| | | Genbank |
|---|---|---|
| Organism | Gene | Accession # |
| Streptomyces coelicolor | fabH1 | NP_626634 |
| | acpfabF | NP_626635 |
| | | NP_626636 |
| Streptomyces avermitilis | fabH3 | NP_823466 |
| | fabC3 (acp) | NP_823467 |
| | fabF | NP_823468 |
| Bacillus subtilis | fabH_A | NP_389015 |
| | fabH_B | NP_388898 |
| | acpfabF | NP_389474 |
| | | NP_389016 |
| Stenotrophomonas | SmalDRAFT_0818 (fabH) | ZP_01643059 |
| maltophilia | SmalDRAFT_0821 (acp) | ZP_01643063 |
| | SmalDRAFT_0822 (fabF) | ZP_01643064 |
| Legionella pneumophila | fabHacpfabF | YP_123672 |
| | | YP_123675 |
| | | YP_123676 |

Expression of the following genes are sufficient to provide cyclohexylcarbonyl-CoA in E. coli: ansJ, ansK, ansL, chcA, and ansM from the ansatrienin gene cluster of Streptomyces collinus (Chen et al., Eur. J Biochem. 261: pp. 1999, 1999) or plmJ, plmK, plmL, chcA, and plmM from the phoslactomycin B gene cluster of Streptomyces sp. HK803 (Palaniappan et al., J. Biol. Chem. 278: pp. 35552, 2003) together with the chcB gene (Patton et al. Biochem., 39: pp. 7595, 2000) from S. collinus, S. avermitilis, or S. coelicolor (see Table 7 for Genbank accession numbers).

TABLE 7
Genes for the synthesis of cyclohexylcarbonyl-CoA

| Organism | Gene | Genbank Accession # |
|---|---|---|
| Streptomyces collinus | ansJK | U72144* |
| | ansL | |
| | chcA | |
| | ansL | |
| | chcB | AF268489 |
| Streptomyces sp. HK803 | pmlJK | AAQ84158 |
| | pmlL | AAQ84159 |
| | chcA | AAQ84160 |
| | pmlM | AAQ84161 |
| Streptomyces coelicolor | chcB/caiD | NP_629292 |
| Streptomyces avermitilis | chcB/caiD | NP_629292 |

Only chcA is annotated in Genbank entry U72144, ansJKLM are according to Chen et al. (Eur. J. Biochem. 261: pp. 1999, 1999)

The genes listed in Table 6 (fabH, ACP and fabF) are sufficient to allow initiation and elongation of co-cyclic fatty acids because they can have broad substrate specificity. In the event that coexpression of any of these genes with the ansJKLM/chcAB or pmlJKLM/chcAB genes from Table 7 does not yield cyFAs, fabH, ACP, and/or fabF homologs from microorganisms that make cyFAs can be isolated (e.g., by using degenerate PCR primers or heterologous DNA probes) and coexpressed. Table 8 lists selected microorganisms that contain co-cyclic fatty acids.

TABLE 8
Examples of microorganisms that contain ω-cyclic fatty acids

| Organism | Reference |
|---|---|
| Curtobacterium pusillum | ATCC19096 |
| Alicyclobacillus acidoterrestris | ATCC49025 |
| Alicyclobacillus acidocaldarius | ATCC27009 |
| Alicyclobacillus cycloheptanicum* | Moore, J. Org. Chem. |
| | 62: pp. 2173, 1997. |

*uses cycloheptylcarbonyl-CoA and not cyclohexylcarbonyl-CoA as precursor for cyFA biosynthesis

As will be appreciated by one of skill in the art, any one or combination of the products discussed above can be incorporated into the fatty esters discussed herein.

Saturation

Production hosts can be engineered to produce unsaturated fatty acids by engineering the production host to over-express fabB or by growing the production host at low temperatures (e.g., less than 37° C.). FabB has a preference for cis-δ³decenoyl-ACP and results in unsaturated fatty acid production in E. coli. Over-expression of fabB resulted in the production of a significant percentage of unsaturated fatty acids (de Mendoza et al., J. Biol. Chem., 258:2098-101, 1983). These unsaturated fatty acids can then be used as intermediates in production hosts that are engineered to produce fatty acid derivatives, such as fatty alcohols, esters, waxes, olefins, alkanes, and the like. One of ordinary skill in the art will appreciate that by controlling the expression of fabA or over-expressing fabB and expressing specific thioesterases (described below), unsaturated fatty acid derivatives having a desired carbon chain length can be produced. Alternatively, the repressor of fatty acid biosynthesis, fabR (Genbank accession NP_418398), can be deleted, which will also result in increased unsaturated fatty acid production in E. coli (Zhang et al., J. Biol. Chem. 277: pp. 15558, 2002). Further increases in unsaturated fatty acids can be achieved by over-expression of fabM (trans-2, cis-3-decenoyl-ACP isomerase, Genbank accession DAA05501) and controlled expression of fabK (trans-2-enoyl-ACP reductase II, Genbank accession NP_357969) from Streptococcus pneumoniae (Marrakchi et al., J. Biol. Chem. 277: 44809, 2002), while deleting E. coli fab I ((trans-2-enoyl-ACP reductase, Genbank accession NP_415804). Additionally, to increase the percentage of unsaturated fatty esters, the microorganism can also have fabB (encoding β-ketoacyl-ACP synthase I, Accessions: BAA16180, EC:2.3.1.41), sfa (encoding a suppressor of fabA, Accession: AAC44390), or gnsA and gnsB (both encoding SecG null mutant suppressors (i.e., cold shock proteins), Accession: ABD18647.1, AAC74076.1) over-expressed.

In some examples, the endogenous fabF gene can be attenuated. This will increase the percentage of palmitoleate (C16:1) produced.

### Processing Enhancement

In some embodiments, the production and isolation of fatty acid derivatives or fatty esters can be enhanced by employing specific processing techniques. One method for increasing production while reducing costs is increasing the percentage of the carbon source that is converted to hydrocarbon products. During normal cellular lifecycles, carbon is used in cellular functions including producing lipids, saccharides, proteins, organic acids, and nucleic acids. Reducing the amount of carbon necessary for growth-related activities can increase the efficiency of carbon source conversion to output. This can be achieved by first growing microorganisms to a desired density, such as a density achieved at the peak of the log phase of growth. At such a point, replication checkpoint genes can be harnessed to stop the growth of cells. Specifically, quorum sensing mechanisms (reviewed in Camilli and Bassler Science 311:1113, 2006; Venturi FEMS Microbio Rev 30:274-291, 2006; and Reading and Sperandio FEMS Microbiol Lett 254:1-11, 2006) can be used to activate genes such as p53, p21, or other checkpoint genes. Genes that can be activated to stop cell replication and growth in E. coli include umuDC genes, the over-expression of which stops the progression from stationary phase to exponential growth (Murli et al., J. of Bact. 182:1127, 2000). UmuC is a DNA polymerase that can carry out translesion synthesis over non-coding lesions, the mechanistic basis of most UV and chemical mutagenesis. The umuDC gene products are used for the process of translesion synthesis and also serve as a DNA damage checkpoint. umuDC gene products include UmuC, UmuD, umuD′, UmuD′₂C, UmuD′₂, and UmuD₂. Simultaneously, the product producing genes would be activated, thus minimizing the need for replication and maintenance pathways to be used while the fatty acid derivative is being made.

The percentage of input carbons converted to hydrocarbon products is a cost driver. The more efficient (i.e., the higher the percentage) the conversion is, the less expensive the process will be. For oxygen-containing carbon sources (i.e. glucose and other carbohydrate based sources), the oxygen must be released in the form of carbon dioxide. For every 2 oxygen atoms released, a carbon atom is also released leading to a maximal theoretical metabolic efficiency of ˜34% (w/w) (for fatty acid derived products). This figure, however, changes for other hydrocarbon products and carbon sources. Typical efficiencies in the literature are less than about 5%. Engineered microorganisms which produce hydrocarbon products can have greater than about 1, 3, 5, 10, 15, 20, 25, and 30% efficiency. In some embodiments, microorganisms will exhibit an efficiency of about 10% to about 25%. In other embodiments, such microorganisms will exhibit an efficiency of about 25% to about 30%, and in other examples such microorganisms will exhibit greater than about 30% efficiency.

In some embodiments, where the final product is released from the cell, a continuous process can be employed. In this approach, a reactor with organisms producing fatty acid derivatives can be assembled in multiple ways. In one example, a portion of the media is removed and let to sit. Fatty acid derivatives are separated from the aqueous layer, which will in turn, be returned to the fermentation chamber.

In one example, the fermentation chamber will enclose a fermentation that is undergoing a continuous reduction. In this instance, a stable reductive environment would be created. The electron balance would be maintained by the release of carbon dioxide (in gaseous form). Efforts to augment the NAD/H and NADP/H balance can also facilitate in stabilizing the electron balance.

The availability of intracellular NADPH can also be enhanced by engineering the production host to express an NADH:NADPH transhydrogenase. The expression of one or more NADH:NADPH transhydrogenases converts the NADH produced in glycolysis to NADPH which enhances the production of fatty acid derivatives.

Disclosed herein is a system for continuously producing and exporting fatty acid derivatives out of recombinant host microorganisms via a transport protein. Many transport and efflux proteins serve to excrete a large variety of compounds and can be evolved to be selective for a particular type of fatty acid derivatives. Thus, in some embodiments an exogenous DNA sequence encoding an ABC transporter will be functionally expressed by the recombinant host microorganism so that the microorganism exports the fatty acid derivative into the culture medium. In one example, the ABC transporter is an ABC transporter from Caenorhabditis elegans, Arabidopsis thalania, Alkaligenes eutrophus, or Rhodococcus erythropolis (locus AAN73268). In another example, the ABC transporter is an ABC transporter chosen from CER5 (locuses At1g51500 or AY734542), AtMRP5, AmiS2, and AtPGP1. In some examples, the ABC transporter is CER5. In yet another example, the CER5 gene is from Arabidopsis (locuses At1g51500, AY734542, At3g21090 and At1g51460).

The transport protein, for example, can also be an efflux protein selected from: AcrAB, TolC, and AcrEF from E. coli, or Tll1618, Tll1619, and Tll0139 from Thermosynechococcus elongatus BP-1.

In addition, the transport protein can be, for example, a fatty acid transport protein (FATP) selected from Drosophila melanogaster, Caenorhabditis elegans, Mycobacterium tuberculosis, or Saccharomyces cerevisiae or any one of the mammalian FATP's. Production hosts can also be chosen for their endogenous ability to release fatty acid derivatives. The efficiency of product production and release into the fermentation broth can be expressed as a ratio of intracellular product to extracellular product. In some examples, the ratio can be 5:1, 4:1, 3:1, 2:1, 1:1, 1:2, 1:3, 1:4, or 1:5.

The production host can be additionally engineered to express recombinant cellulosomes, such as those described in PCT application number PCT/US2007/003736, which will allow the production host to use cellulosic material as a carbon source. For example, the production host can be additionally engineered to express invertases (EC 3.2.1.26) so that sucrose can be used as a carbon source.

Similarly, the production host can be engineered using the teachings described in U.S. Pat. Nos. 5,000,000, 5,028,539, 5,424,202, 5,482,846, and 5,602,030 to Ingram et al. so that the production host can assimilate carbon efficiently and use cellulosic materials as carbons sources.

### Post Production Processing

The fatty acid derivatives or fatty esters produced during production can be separated from the production media. Any technique known for separating fatty acid derivatives or fatty esters from aqueous media can be used. One exemplary separation process provided herein is a two phase (bi-phasic) separation process. This process involves processing the genetically engineered production hosts under conditions sufficient to produce a fatty acid derivative (e.g., a fatty ester), allowing the derivative to collect in an organic phase and separating the organic phase from the aqueous production broth. This method can be practiced in both a batch and continuous production setting.

Bi-phasic separation uses the relative immisiciblity of fatty acid derivatives to facilitate separation. Immiscible refers to the relative inability of a compound to dissolve in water and is defined by the compound's partition coefficient. The partition coefficient, P, is defined as the equilibrium concentration of a compound in an organic phase (in a bi-phasic system the organic phase is usually the phase formed by the fatty acid derivative) during the production process. However, in some examples an organic phase can be provided (e.g., a layer of octane to facilitate product separation)) divided by the concentration at equilibrium in an aqueous phase (i.e., production broth). When describing a two phase system the P is usually discussed in terms of log P. A compound with a log P of 1 would partition 10:1 to the organic phase, while a compound of log P of 0.1 would partition 1:10 to the organic phase. One or ordinary skill in the art will appreciate that by choosing a production broth and the organic phase such that the fatty acid derivative being produced has a high log P value, the fatty acid derivative will separate into the organic phase, even at very low concentrations in the production vessel.

The fatty acid derivatives produced by the methods described herein will be relatively immiscible in the production broth, as well as in the cytoplasm. Therefore, the fatty acid derivative will collect in an organic phase either intracellularly or extracellularly. The collection of the products in an organic phase will lessen the impact of the fatty acid derivative on cellular function and will allow the production host to produce more product. Stated another way, the concentration of the fatty acid derivative will not have as significant of an impact on the host cell.

The fatty esters produced as described herein allow for the production of homogeneous compounds wherein at least about 60%, 70%, 80%, 90%, 95%, 98%, 99%, or 100% by volume of the fatty esters produced will have carbon chain lengths that vary by less than about 4 carbons or less than about 2 carbons. These compounds can also be produced so that they have a relatively uniform degree of saturation, for example at least about 60%, 70%, 80%, 90%, 95%, 98%, 99%, or 100% by volume of the fatty esters will be mono-, di-, or tri-unsaturated. These compounds can be used directly as fuels, personal care additives, or nutritional supplements. These compounds can also be used as feedstock for subsequent reactions, for example transesterification, hydrogenation, catalytic cracking via either hydrogenation, pyrolisis, or both or epoxidations reactions, to make other products. The fatty esters can also be concentrated such that the composition of which they are part will comprise at least about 80% fatty esters, for example, the percent fatty ester can be about 80-85, 85-90, 90-95, 95-99% or more.

In some embodiments, in order to be used as a biofuel, the fatty ester composition can be further processed. In some embodiments, the fatty ester composition can be isolated from the broth and the cells. In addition, the fatty ester composition can be purified to remove excess water. In some embodiments, fine solids can be removed that might affect injection nozzles or prefilters in engines. In some embodiments, the fatty ester composition can also be processed to remove species that have poor volatility and would lead to deposit formation. In some embodiments, traces of sulfur compounds that may be present are removed. In some embodiments, the above can be achieved via one or more of the following: washing, adsorption, distillation, filtration, centrifugation, settling, and coalescence.

In some embodiments, during processing, impurities in the alcohol can enter the fermentation off gas. Off gas treatment steps can be used as appropriate depending on the impurity.

### Reduced Impurities

In some embodiments, the fatty acid derivatives described herein can be useful for making biofuels. In some embodiments, these fatty acid derivatives are made directly from fatty acids. Accordingly, in some embodiments, fuels comprising the disclosed fatty acid derivatives can contain less of some types of impurities that are normally associated with biofuels derived from triglycerides, such as fuels derived from vegetable oils and fats.

The crude fatty acid derivative biofuels described herein (prior to mixing the fatty acid derivative with other fuels, such as traditional fuels) will contain less transesterification catalyst than petrochemical diesel or biodiesel. For example, the fatty acid derivative can contain less than about 2%, 1.5%, 1%, 0.5%, 0.3%, 0.1%, 0.05%, or 0% by volume of a transesterification catalyst or an impurity resulting from a transesterification catalyst. Transesterification catalysts include, for example, hydroxide catalysts, such as NaOH, KOH, LiOH, and acidic catalysts, such as mineral acid catalysts and Lewis acid catalysts. Catalysts and impurities resulting from transesterification catalysts include, without limitation, tin, lead, mercury, cadmium, zinc, titanium, zirconium, hafnium, boron, aluminum, phosphorus, arsenic, antimony, bismuth, calcium, magnesium, strontium, uranium, potassium, sodium, lithium, and combinations thereof.

Similarly, the crude fatty acid derivative biofuels described herein (prior to mixing the fatty acid derivative with other fuels such as petrochemical diesel or biodiesel) will contain less glycerol (or glycerin) than bio-fuels made from triglycerides. For example, the fatty acid derivative can contain less than about 2%, 1.5%, 1%, 0.5%, 0.3%, 0.1%, 0.05%, or 0% glycerol.

The crude biofuel derived from fatty acid derivatives will also contain less free alcohol (i.e., alcohol that is used to create the ester) than biodiesel made from triglycerides. This is, in part, due to the efficiency of utilization of the alcohol by the production host. For example, the fatty acid derivative will contain less than about 2%, 1.5%, 1%, 0.5%, 0.3%, 0.1%, 0.05%, or 0% free alcohol.

Biofuel derived from the disclosed fatty acid derivatives can be additionally characterized by its low concentration of sulfur compared to petroleum derived diesel. For example, biofuel derived from fatty acid derivatives can have less than about 2%, 1.5%, 1%, 0.5%, 0.3%, 0.1%, 0.05%, or 0% sulfur.

In some embodiments, while the biofuel, fatty ester, or fatty ester derivative has less of one or more of the above impurities, it has more of another impurity. For example, the biofuel, fatty ester, or fatty acid derivative can have additional impurities from those of unrefined or impure alcohols (e.g., ethanol) as noted above. Thus, in some embodiments, the biofuel, fatty ester, or fatty acid derivative can have more of some types of impurities (e.g., those present in an impure alcohol) and less of the impurities discussed within this section.

### Fuel Compositions

The fatty esters and combinations thereof described herein can be used as a fuel. One of ordinary skill in the art will appreciate that depending upon the intended purpose of the fuel, different fatty esters can be produced and used. For example, for automobile fuel that is intended to be used in cold climates, a branched fatty ester can be desirable. Using the teachings provided herein, branched hydrocarbons, fatty esters, and alcohols can be made. Using the methods described herein, fuels comprising relatively heterogenous fatty acid derivatives that have desired fuel qualities can be produced. Such fuels can be characterized by carbon fingerprinting or their lack of impurities when compared to petroleum derived fuels or biodiesel derived from triglycerides. Moreover, the fatty ester based fuels can be combined with other fuels or fuel additives to produce fuels having desired properties.

In some embodiments, the fatty ester composition comprises a variety of fatty esters that can vary in Aₙ and Bₙ length, saturation level, and ratios between the different species. Thus, in some embodiments, Bₙ can be a 6, 7, 8, 9, 10, 11, 12, 13, 14, 15, 16, 17, 18, 19, 20, 21, 22, 23, 24, 25, 26, 27, 28, 29, or 30 carbon chain which can have 1, 2, 3, 4, 5, 6, 7, 8, 9, 10, 11, 12, 13, 14, 15, 16, 17, 18, 19, 20, 21, 22, 23, or 24 double bonds. 1-24 of those double bonds can be located following carbon 1, 2, 3, 4, 5, 6 7, 8, 9, 10, 11, 12, 13, 14, 15, 16, 17, 18, 19, 20, 21, 22, 23, 24, 25, 26, 27, 28, or 29. Aₙ can be a 1, 2, 3, 4, 5, or 6 carbon chain having 1, 2, 3, 4, or 5 double bonds. 1-5 of those double bonds can be located following carbon 1, 2, 3, 4, or 5. One or more of these AₙCOOBₙ species (each different species denoted as A₁COOB₁, A₂COOB₂, A₃COOB₃, etc.) can make up some fraction of the fatty ester composition. Thus, in some embodiments, one or more of the above species makes up at least about 1%, 5%, 10%, 15%, 20%, 25%, 30%, 35%, 40%, 50%, 60%, 70%, 80%, 90%, or 95% by volume of the fatty ester composition. In some embodiments, the fatty ester composition is at least about 50 to about 95 wt % C_{16:1} ethyl ester, at least about 50 to about 95 wt % C_{18:1} ethyl ester, at least about 50 to about 95 wt % C_{16:0} ethyl ester, and/or at least about 50 to about 95 wt % C_{18:0} ethyl ester. In some embodiments, the fatty ester composition is at least about 50 to about 100 wt % C_{16:1} ethyl ester, at least about 50 to about 100 wt % C_{18:1} ethyl ester, at least about 50 to about 100 wt % C_{16:0} ethyl ester, and/or at least about 50 to about 100 wt % C_{18:0} ethyl ester. In some embodiments, the fatty ester composition is at least about 50 to about 95 wt % C_{16:1} ester, at least about 50 to about 95 wt % C_{18:1} ester, at least about 50 to about 95 wt % C_{16:0} ester, and/or at least about 50 to about 95 wt % C_{18:0} ester. In some embodiments, the fatty ester composition is at least about 50 to about 100 wt % C_{16:1} ester, at least about 50 to about 100 wt % C_{18:1} ester, at least about 50 to about 100 wt % C_{16:0} ester, and/or at least about 50 to about 100 wt % C_{18:0} ester. In some embodiments, the fatty ester composition is at least about 50 to about 95 wt % C_{16:1} methyl ester, at least about 50 to about 95 wt % C_{18:1} methyl ester, at least about 50 to about 95 wt % C_{16:0} methyl ester, and/or at least about 50 to about 95 wt % C_{18:0} methyl ester. In some embodiments, the fatty ester composition is at least about 50 to about 100 wt % C_{16:1} methyl ester, at least about 50 to about 100 wt % C_{18:1} methyl ester, at least about 50 to about 100 wt % C_{16:0} methyl ester, and/or at least about 50 to about 100 wt % C_{18:0} methyl ester. In some embodiments, the fatty ester composition comprises about 20%, 30%, 40%, 50%, 60%, 70%, 80%, 85%, 90%, or 95% fatty ester that has a Bₙ carbon chain that is 8:0, 10:0, 12:0, 14:0, 14:1, 16:0, 16:1, 18:0, 18:1, 18:2, 18:3, 20:0, 20:1, 20:2, 20:3, 22:0, 22:1, or 22:3.

### Additives

In some embodiments, fuel additives are used to enhance the performance of a fuel or engine. For example, fuel additives can be used to alter the freezing/gelling point, cloud point, lubricity, viscosity, oxidative stability, ignition quality, octane level, and flash point. In the United States, all fuel additives must be registered with the Environmental Protection Agency (EPA). Companies that sell fuel additives and the name of the fuel additive are publicly available on the EPA's website or also by contacting the EPA. One of ordinary skill in the art will appreciate that the fatty acid derivatives described herein can be mixed with one or more such additives to impart a desired quality.

One of ordinary skill in the art will also appreciate that the fatty acid derivatives described herein can be mixed with other fuels, such as biodiesel derived from triglycerides, various alcohols, such as ethanol and butanol, and petroleum derived products, such as diesel or gasoline. In some examples, a fatty acid derivative, such as C16:1 ethyl ester or C18:1 ethyl ester, is produced which has a low gel point. This low gel point fatty acid derivative is mixed with biodiesel made from triglycerides to lessen the overall gelling point of the fuel. Similarly, a fatty acid derivative, such as C16:1 ethyl ester or C18:1 ethyl ester, can be mixed with petroleum derived diesel to provide a mixture that is at least and often greater than 5% biodiesel. In some examples, the mixture includes at least about 20% or greater of the fatty acid derivative.

For example, a biofuel composition can be made that includes at least about 20%, 30%, 40%, 50%, 60%, 70%, 80%, 85%, 90% or 95% by volume of a fatty acid derivative and/or fatty ester that includes a carbon chain that is 8:0, 10:0, 12:0, 14:0, 14:1, 16:0, 16:1, 18:0, 18:1, 18:2, 18:3, 20:0, 20:1, 20:2, 20:3, 22:0, 22:1 or 22:3. Such biofuel compositions can additionally include at least one additive selected from a cloud point lowering additive that can lower the cloud point to less than about 5° C., or 0° C., a surfactant, or a microemulsion, at least about 5%, 10%, 15%, 20%, 30%, 40%, 50%, 60%, 70% or 80%, 85%, 90%, or 95% diesel fuel from triglycerides, petroleum derived gasoline or diesel.

In some embodiments, the above method or composition can further include the addition of one or more fuel additives. As noted above, in some embodiments, additional amounts of a second (or more) fatty ester can be added to the resulting fatty ester mixture. In some embodiments, the additional fatty ester is different from any of the fatty esters in the resulting fatty ester mixture produced by the production process. In some embodiments, the additional fatty ester is the same as one of the fatty esters present in the resulting fatty ester mixture, but the additional fatty ester can alter the amount of the fatty ester present in the resulting fatty ester mixture.

As will be appreciated by one of skill in the art, any of the above fatty esters and fatty ester compositions can be converted into a biofuel, or more specifically biodiesel, if desired. Thus, the corresponding biofuels and biodiesels are also provided herein.

### ADDITIONAL EMBODIMENTS

In some embodiments, an additional advantage of a production host system is the ability to produce primarily or only saturated and monounsaturated fatty esters. In contrast, plant oils are rich in di- and tri-unsaturated FAs, which are less stable to oxygen, resulting in significant handling and storage constraints.

In some embodiments, the method comprises employing methanol and at least one different alcohol having a different number of carbon atoms from methanol, wherein the mixture substantially lacks propanol. Using this mixture, one can produce fatty esters by providing the mixture to a fatty ester production host. In some embodiments, the use of methanol results in a total amount of fatty ester produced that is greater than an amount of fatty ester that is produced when the methanol is replaced with a different alcohol. In some embodiments, the amount of free fatty acids that results from the method is less than an amount of free fatty acid produced when the at least one different alcohol is used without methanol.

In some embodiments, the method comprises selecting methanol as a first alcohol for an alcohol mixture, selecting a second alcohol for the alcohol mixture, providing the alcohol mixture to a fatty ester production host, and converting the alcohols of the alcohol mixture to a fatty ester composition using the fatty ester production host. The presence of methanol in the alcohol mixture results in a fatty ester where A₁ is an alkyl group of 1 carbon in length and the fatty ester composition is biased to include more fatty esters having Bₙ selected from the group consisting of C16, 17, C18, and any combination thereof, in comparison to a method wherein the only alcohol is ethanol.

In some embodiments, a method of producing a fatty ester composition is provided that comprises selecting ethanol as a first alcohol for an alcohol mixture, selecting a second alcohol for the alcohol mixture, providing the alcohol mixture to a fatty ester production host, and converting the alcohols of the alcohol mixture to a fatty ester composition using the fatty ester production host. In some embodiments, the fatty ester composition is biased to include more fatty esters having Bₙ selected from the group consisting of C12, 13, C14, and any combination thereof, in comparison to a method wherein the only alcohol is methanol.

In some embodiments, the combined fatty esters will include at least about 50 to about 100 wt % C_{16:1} ethyl ester, at least about 50 to about 100 wt % C_{18:1} ethyl ester, at least about 50 to about 100 wt % C_{16:0} ethyl ester, and/or at least about 50 to about 100% C_{18:0} ethyl ester. In some embodiments, the product is at least about 50 to about 95 wt % C_{16:1} ethyl ester, at least about 50 to about 95 wt % C_{18:1} ethyl ester, at least about 50 to about 95 wt % C_{16:0} ethyl ester, and/or at least about 50 to about 95% C_{18:0} ethyl ester. In some embodiments, the combined fatty esters will include at least about 50 to about 100 wt % C_{16:1} ester, at least about 50 to about 100 wt % C_{18:1} ester, at least about 50 to about 100 wt % C_{16:0} ester, and/or at least about 50 to about 100% C_{18:0} ester. In some embodiments, the product is at least about 50 to about 95 wt % C_{16:1} ester, at least about 50 to about 95 wt % C_{18:1} ester, at least about 50 to about 95 wt % C_{16:0} ester, and/or at least about 50 to about 95% C_{18:0} ester. In some embodiments, the combined fatty esters will include at least about 50 to about 100 wt % C_{16:1} methyl ester, at least about 50 to about 100 wt % C_{18:1} methyl ester, at least about 50 to about 100 wt % C_{16:0} methyl ester, and/or at least about 50 to about 100% C_{18:0} methyl ester. In some embodiments, the product is at least about 50 to about 95 wt % C_{16:1} methyl ester, at least about 50 to about 95 wt % C_{18:1} methyl ester, at least about 50 to about 95 wt % C_{16:0} methyl ester, and/or at least about 50 to about 95% C_{18:0} methyl ester.

### EXAMPLES

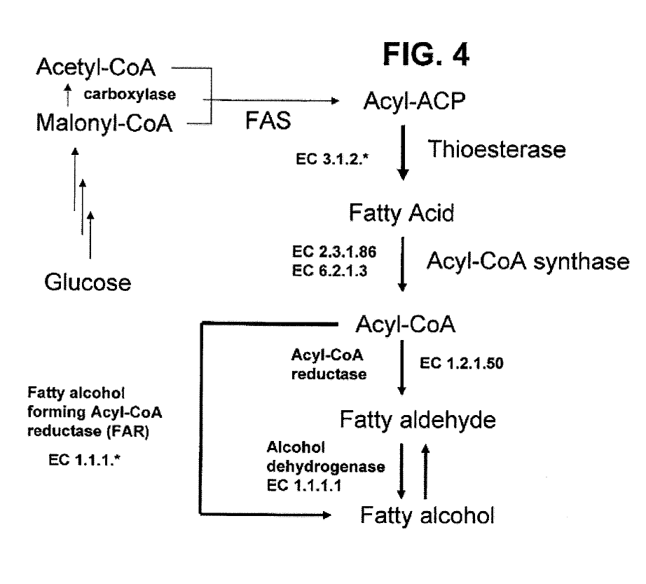

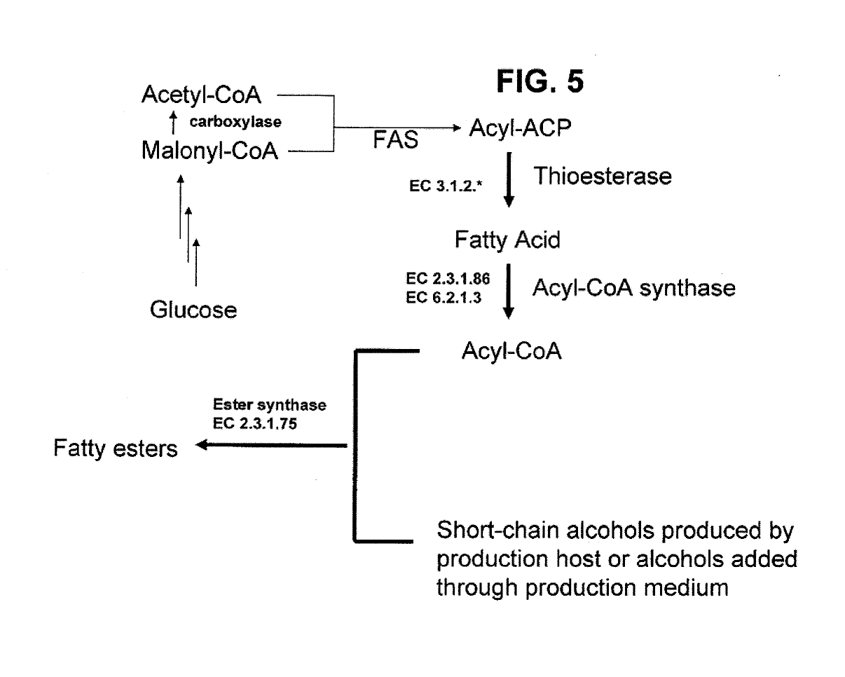

The examples provided herein illustrate the engineering of production hosts to produce specific fatty acid derivatives. Exemplary biosynthetic pathway involved in the production of fatty acid derivatives and fatty esters are illustrated in the figures. For example, FIG. 2 is a diagram of the FAS pathway showing the enzymes directly involved in the synthesis of acyl-ACP. To increase the production of fatty acid derivatives, such as waxes, fatty esters, fatty alcohols, and hydrocarbons one or more of the enzymes in FIG. 2 can be over expressed or mutated to reduce feedback inhibition to increase the amount of acyl-ACP produced. Additionally, enzymes that metabolize the intermediates to make non-fatty acid based products (side reactions) can be functionally deleted or attenuated to increase the flux of carbon through the fatty acid biosynthetic pathway. In the examples below, many production hosts are described that have been modified to increase fatty acid production. FIG. 3, FIG. 4, and FIG. 5 show biosynthetic pathways that can be engineered to make fatty alcohols and fatty esters, respectively. As illustrated in FIG. 3, the conversion of each substrate (e.g., acetyl-CoA, malonyl-CoA, acyl-ACP, fatty acid, and acyl-CoA) to each product (e.g., acetyl-CoA, malonyl-CoA, acyl-ACP, fatty acid, and acyl-CoA) can be accomplished using several different polypeptides that are members of the enzyme classes indicated.

The examples below describe microorganisms that have been engineered or can be engineered to produce specific fatty alcohols, waxes, fatty esters, and hydrocarbons.

### Example 1

### Production Host Construction

The present example outlines various production hosts and methods of making them. An exemplary production host is LS9001. LS9001 was produced by modifying C41(DE3) from Overexpress (Saint Beausine, France) to knock-out the fadE gene (acyl-CoA dehydrogenase).

Briefly, the fadE knock-out strain of E. coli was made using primers YafV_NotI and Ivry_O1 to amplify about 830 bp upstream of fadE and primers Lpcaf_ol and LpcaR_Bam to amplify about 960 bp downstream of fadE. Overlap PCR was used to create a construct for in-frame deletion of the complete fadE gene. The fadE deletion construct was cloned into the temperature-sensitive plasmid pKOV3, which contained a sacB gene for counterselection, and a chromosomal deletion of fadE was made according to the method of Link et al., J. Bact. 179:6228-6237, 1997. The resulting strain was not capable of degrading fatty acids and fatty acyl-CoAs. This knock-out strain is herein designated as E. coli (DE3, ΔfadE).

Another fadE deletion strain, MG1655 was constructed exactly according to Baba et al, Mol Syst Bio 2:1-11, 2006 and used to produce fatty alkyl esters. This E. coli strain is designated as MG1655 (ΔfadE).

An additional production host that is made included the following adjustments: fabH/fabD/fabG/acpP/fabF (encoding enzymes involved in fatty acid biosynthesis) from E. coli, Nitrosomonas europaea (ATCC 19718), Bacillus subtilis, Lactobacillus plantarum Saccharomyces cerevisiae, Streptomyces spp, Ralstonia, Rhodococcus, Corynebacteria, Brevibacteria, Mycobacteria, and oleaginous yeast.

Similarly, production hosts were engineered to express accABCD (encoding acetyl co-A carboxylase) from Lactobacillus plantarum in the E. coli host with fadE deleted.

In some production hosts, genes were knocked out or attenuated using the method of Link, et al., J. Bacteriol. 179:6228-6237, 1997. Genes that were knocked out or attenuated include ldhA (encoding lactate dehydrogenase, accession NP_415898, EC: 1.1.1.28); pta (encoding phosphotransacetylase, accessions: NP_416800, EC: 2.3.1.8); poxB (encoding pyruvate oxidase, accessions: NP_415392, EC: 1.2.2.2); ackA (encoding acetate kinase, accessions: NP_416799, EC: 2.7.2.1); fabR (encoding a transcription dual regulator, accession number U00096.2) and combinations thereof.

Additional gene deletions may benefit to optimum production of fatty esters are listed the Table 9.

TABLE 9

| Enzymatic activity | EC number | E. coli gene |
|---|---|---|
| Acyl-ACP synthase | 6.2.1.20, 2.3.1.40 | aaS |
| Lactate dehydrogenase | none | dld |
| Lactate dehydrogenase | 1.1.2.4 | lld |
| Ethanol dehydrogenase | 1.1.1.1 | adhP |

For the commercial production of fatty acid derivatives via fermentation, the production host internal regulatory pathways were optimized to produce more of the desired products. In many instances, this regulation is diminished by overexpressing certain enzymes.

Additional examples of certain enzymes that can be overexpressed in various embodiments are shown in Table 10.

TABLE 10
Additional genes that can be optimized for fatty acid derivative production

| | | Example of E. coli |
|---|---|---|
| | EC | gene(s) (or other |
| Enzymatic Activity | Number | microorganism) |
| Pantetheine-phosphate adenylyltransferase | 2.7.7.3 | coaD |
| Dephospho-CoA kinase | 2.7.1.24 | coaE |
| Pantetheinate kinase | 2.7.1.33 | coaA(panK) |
| Biotin-[acetyl-CoA-carboxylase] ligase | 6.3.4.15 | birA |
| Carbonic anhydrase | 4.2.1.1 | cynT, can(yadF) |
| Apo-[acyl carrier protein] | None | acpP |
| Holo-[acyl-carrier-protein] synthase | 2.7.8.7 | acpS, acpT |
| Pyruvate dehydogenase complex | 1.2.4.1 | aceF |
| | 2.3.1.12 | aceE |
| | 1.8.1.4 | lpd |
| NAD Kinase | 2.7.1.23 | nadK (yfjB) |
| Pyruvate-ferredoxin oxidoreductase | 1.2.7.1 | porA (Desulfovobrio |
| | | vulgaris DP4) |

### Example 2

### Additional Production Hosts

The present example outlines additional modifications that can be made to various production hosts.

The following plasmids were constructed for the expression of various proteins that are used in the synthesis of fatty acid derivatives. The constructs were made using standard molecular biology methods. The cloned genes were put under the control of IPTG-inducible promoters (e.g., T7, tac, or lac promoters).

The ′tesA gene (thioesterase A gene accession NP_415027 without leader sequence (Cho and Cronan, J. Biol. Chem., 270:4216-9, 1995, EC: 3.1.1.5, 3.1.2.-)) of E. coli was cloned into NdeI/AvrII digested pETDuet-1 (pETDuet-1 described herein is available from Novagen, Madison, Wis.). Genes encoding for FatB-type plant thioesterases (TEs) from Umbellularia californica, Cuphea hookeriana, and Cinnamonum camphorum (accessions: UcFatB1=AAA34215, ChFatB2=AAC49269, ChFatB3=AAC72881, CcFatB=AAC49151) were individually cloned into three different vectors: (i) NdeI/AvrII digested pETDuet-1; (ii)XhoI/HindIII digested pBluescript KS+ (Stratagene, La Jolla, Calif., to create N-terminal lacZ::TE fusion proteins); and (iii) XbaI/HindIII digested pMAL-c2X (New England Lab, Ipswich, Mass.) (to create n-terminal malE::TE fusions). The fadD gene (encoding acyl-CoA synthase) from E. coli was cloned into a NcoI/HindIII digested pCDFDuet-1 derivative, which contained the acr1 gene (acyl-CoA reductase) from Acinetobacter baylyi ADP1 within its NdeI/AvrII sites. Table 11 provides a summary of the plasmids generated to make several exemplary production hosts.

The chosen expression plasmids contained compatible replicons and antibiotic resistance markers to produce a four-plasmid expression system.

TABLE 11
Summary of plasmids used in production hosts

| | Source Organism | Accession No., |
|---|---|---|
| Plasmid | Gene Product | EC number |
| pETDuet-1-tesA | E. coli | Accessions: NP_415027, |
| | TesA (without leader | EC: 3.1.1.5, 3.1.2.— |
| | sequence) | |
| pETDuet-1-TEuc | Umbellularia californica | Q41635 |
| pBluescript-TEuc | UcFatB1 | |
| pMAL-c2X-TEuc | | AAA34215 |
| pETDuet-1-Tech | Cuphea hookeriana | ABB71581 |
| pBluescript-TEch | ChFatB2 | AAC49269 |
| pMAL-c2X-Tech | ChFatB3 | AAC72881 |
| pETDuet-1-TEcc | Cinnamonum camphorum | |
| pBluescript-TEcc | CcFabB | AAC49151 |
| TEci | | |
| pETDuet-1- | Arabidopsis thaliana | NP_189147 |
| atFatA3 | | |
| pETDuet-1- | Helianthus annuus | AAL769361 |
| HaFatA1 | | |
| pCDFDuet-1- | fadD from E. coli an acr1 | fadD: Accessions |
| fadD-acr1 | from Acinetobacter baylyi | NP_416319, EC 6.2.1.3 |
| | ADP1 | acr1: Accessions |
| | | YP_047869 |

One of ordinary skill in the art will appreciate that different plasmids and genomic modifications can be used to achieve similar strains to those noted in this example.

In some embodiments, LS9001 can be co-transformed with: (i) any of the TE-expressing plasmids; (ii) the FadD-expressing plasmid, which also expresses Acr1; and (iii) ester synthase expression plasmid.

As will be clear to one of skill in the art, when LS9001 is induced with IPTG, the resulting strain will produce increased concentrations of fatty alcohols from carbon sources such as glucose.

### Example 3

### Medium Chain Fatty Esters

Alcohol acetyl transferases (AATs, EC 2.3.1.84), which is responsible for acyl acetate production in various plants, can be used to produce medium chain length fatty esters, such as octyl octanoate, decyl octanoate, decyl decanoate, and the like. An AAT gene can be inserted into one of the production hosts described herein by by the methods noted in the above examples.

As will be appreciated by one of skill in the art, fatty esters, synthesized from medium chain alcohol (such as C₆ and C₈) and medium chain acyl-CoA (or fatty acids, such as C₆ and C₈) have a relativly low melting point. For example, hexyl hexanoate has a melting point of −55° C. and octyl octanoate has a melting point of −18° C. to −17° C. The low melting points of these compounds make them good candidates for use as biofuels.

### Example 4

### Production and Release of Fatty Ethyl Ester from Production Host

The present example outlines how to produce a fatty ester by using a LS9001 production host.

The LS9001 strain was transformed with plasmids carrying an ester synthase gene from A. baylyi ADP1 (plasmid pHZ1.43), a thioesterase gene from Cuphea hookeriana (plasmid pMAL-c2X-TEch), and a fadD gene from E. coli (plasmid pCDFDuet-1-fadD).

Plasmid pHZ1.43 carrying the wax synthase (WSadp1, accessions AA017391, EC 2.3.175) was constructed as follows. First the gene for WSadp1 was amplified with the following primers using genomic DNA sequence from A. baylyi ADP1 as the template: (1) WSadp1_NdeI, 5′-TCATATGCGCCCATTACATCCG-3′ and (2) WSadp1_Avr, 5′-TCCTAGGAGGGCTAATTTAGCCCTTTAGTT-3′. Then PCR product was digested with NdeI and AvrII and cloned into pCOALDeut-1 to give pHZ1.43

This recombinant strain was grown at 25° C. in 3 mL M9 medium with 50 mg/L kanamycin, 100 mg/L carbenicillin, and 100 mg/L of spectinomycin. After IPTG induction, the media was adjusted to a final concentration of 1% ethanol and 2% glucose.

The culture was allowed to grow for 40 hours after IPTG induction. The cells were separated from the spent medium by centrifugation at 3500×g for 10 minutes. The cell pellet was re-suspended with 3 mL of M9 medium. The cell suspension and the spent medium were then extracted with 1 volume of ethyl acetate. The resulting ethyl acetate phases from the cell suspension and the supernatant were subjected to GC-MS analysis.

The C₁₆ ethyl ester was the most prominent ester species (as expected for this thioesterase, see Table 3), and 20% of the fatty ester produced was released from the cell (see FIG. 6). A control E. coli strain C41(DE3, ΔfadE) containing pCOLADuet-1 (empty vector for the ester synthase gene), pMAL-c2X-TEuc (containing fatB from U. california) and pCDFDuet-1-fadD (fadD gene from E. coli) failed to produce detectable amounts of fatty ethyl esters. The fatty esters were quantified using commercial palmitic acid ethyl ester as the reference.

Fatty esters were also made using the methods described herein except that methanol or isopropanol was added to the production broth. The predicted fatty esters were produced.

### Example 5

### Alternative Production Hosts and Uses Thereof

The present example examines the influence of various thioesterases on the composition of fatty-ethyl esters produced in recombinant E. coli strains.

The thioesterases FatB3 (C. hookeriana), ′TesA (E. coli), and FatB (U. california) were expressed simultaneously with ester synthase (A. baylyi). A plasmid, pHZ1.61, was constructed by replacing the NotI-AvrII fragment (carrying the acr1 gene) with the NotI-AvrII fragment from pHZ1.43 so that fadD and the ADP1 ester synthase were in one plasmid and both coding sequences were under the control of separate T7 promoters. The construction of pHZ1.61 made it possible to use a two plasmid system instead of the three plasmid system. pHZ1.61 was then co-transformed into E. coli C41(DE3, ΔfadE) with one of the various plasmids carrying the different thioesterase genes stated above.

The total fatty ethyl esters (in both the supernatant and intracellular fatty ethyl fluid) produced by these transformants were evaluated using the technique described herein. The yields and the composition of fatty ethyl esters are summarized in Table 12. In regard to Table 9, the following is noted: ′TesA, pETDuet-1-′tesA; chFatB3, pMAL-c2X-TEch; ucFatB, pMAL-c2X-TEuc; pMAL, pMAL-c2X, the empty vector for thioesterase genes used in the study.

TABLE 12

| Thioesterases | C₂C₁₀ | C₂C_{12:1} | C₂C₁₂ | C₂C_{14:1} | C₂C₁₄ | C₂C_{16:1} | C₂C₁₆ | C₂C_{18:1} | Total |
|---|---|---|---|---|---|---|---|---|---|
| ‘TesA | 0.0 | 0.0 | 6.5 | 0.0 | 17.5 | 6.9 | 21.6 | 18.1 | 70.5 |
| ChFatB3 | 0.0 | 0.0 | 0.0 | 0.0 | 10.8 | 12.5 | 11.7 | 13.8 | 48.8 |
| ucFatB | 6.4 | 8.5 | 25.3 | 14.7 | 0.0 | 4.5 | 3.7 | 6.7 | 69.8 |
| pMAL-c2x | 0.0 | 0.0 | 0.0 | 0.0 | 5.6 | 0.0 | 12.8 | 7.6 | 26.0 |

### Example 6

### Production Host Construction

The present example outlines various genes that can be manipulated in a production host as well as providing additional production hosts.

Table 13 identifies the homologues of many of the genes described herein that are expressed in microorganisms that produce biodiesels, fatty alcohols, and hydrocarbons. To increase fatty acid production and, therefore, hydrocarbon production in production hosts such as those identified in Table 13, heterologous genes can be expressed, such as those from E. coli.

Any one or more of the genes listed in Table 13 can be manipulated (e.g., added, attenuated, overexpressed, or removed) in any desired production host (including those in Table 13). The genes that are endogenous to the micoorganisms provided in Table 13 can be expressed, over-expressed, or attenuated using the methods described herein. In addition, the genes that are described in Table 13 can be expressed, overexpressed, removed, or attenuated in a production host that endogenously produce hydrocarbons to allow for the production of specific hydrocarbons with defined carbon chain length, saturation points, and branch points. The resulting production hosts can be used as described herein.

TABLE 13
Hydrocarbon production hosts

| Organism | Gene Name | Accession No./Seq ID/Loci | EC No. |
|---|---|---|---|
| Desulfovibrio desulfuricans | accA | YP_388034 | 6.4.1.2 |
| G20 | | | |
| Desulfovibrio desulfuricans | accC | YP_388573/YP_388033 | 6.3.4.14, 6.4.1.2 |
| G22 | | | |
| Desulfovibrio desulfuricans | accD | YP_388034 | 6.4.1.2 |
| G23 | | | |
| Desulfovibrio desulfuricans | fabH | YP_388920 | 2.3.1.180 |
| G28 | | | |
| Desulfovibrio desulfuricans | fabD | YP_388786 | 2.3.1.39 |
| G29 | | | |
| Desulfovibrio desulfuricans | fabG | YP_388921 | 1.1.1.100 |
| G30 | | | |
| Desulfovibrio desulfuricans | acpP | YP_388922/YP_389150 | 3.1.26.3, |
| G31 | | | 1.6.5.3, 1.6.99.3 |
| Desulfovibrio desulfuricans | fabF | YP_388923 | 2.3.1.179 |
| G32 | | | |
| Desulfovibrio desulfuricans | gpsA | YP_389667 | 1.1.1.94 |
| G33 | | | |
| Desulfovibrio desulfuricans | ldhA | YP_388173/YP_390177 | 1.1.1.27, |
| G34 | | | 1.1.1.28 |
| Erwinia (micrococcus) | accA | 942060-943016 | 6.4.1.2 |
| amylovora | | | |
| Erwinia (micrococcus) | accB | 3440869-3441336 | 6.4.1.2 |
| amylovora | | | |
| Erwinia (micrococcus) | accC | 3441351-3442697 | 6.3.4.14, 6.4.1.2 |
| amylovora | | | |
| Erwinia (micrococcus) | accD | 2517571-2516696 | 6.4.1.2 |
| amylovora | | | |
| Erwinia (micrococcus) | fadE | 1003232-1000791 | 1.3.99.— |
| amylovora | | | |
| Erwinia (micrococcus) | plsB(D311E) | 333843-331423 | 2.3.1.15 |
| amylovora | | | |
| Erwinia (micrococcus) | aceE | 840558-843218 | 1.2.4.1 |
| amylovora | | | |
| Erwinia (micrococcus) | aceF | 843248-844828 | 2.3.1.12 |
| amylovora | | | |
| Erwinia (micrococcus) | fabH | 1579839-1580789 | 2.3.1.180 |
| amylovora | | | |
| Erwinia (micrococcus) | fabD | 1580826-1581749 | 2.3.1.39 |
| amylovora | | | |
| Erwinia (micrococcus) | fabG | CAA74944 | 1.1.1.100 |
| amylovora | | | |
| Erwinia (micrococcus) | acpP | 1582658-1582891 | 3.1.26.3, |
| amylovora | | | 1.6.5.3, 1.6.99.3 |
| Erwinia (micrococcus) | fabF | 1582983-1584221 | 2.3.1.179 |
| amylovora | | | |
| Erwinia (micrococcus) | gpsA | 124800-125810 | 1.1.1.94 |
| amylovora | | | |
| Erwinia (micrococcus) | ldhA | 1956806-1957789 | 1.1.1.27, |
| amylovora | | | 1.1.1.28 |
| Kineococcus radiotolerans | accA | ZP_00618306 | 6.4.1.2 |
| SRS30216 | | | |
| Kineococcus radiotolerans | accB | ZP_00618387 | 6.4.1.2 |
| SRS30216 | | | |
| Kineococcus radiotolerans | accC | ZP_00618040/ | 6.3.4.14, 6.4.1.2 |
| SRS30216 | | ZP_00618387 | |
| Kineococcus radiotolerans | accD | ZP_00618306 | 6.4.1.2 |
| SRS30216 | | | |
| Kineococcus radiotolerans | fadE | ZP_00617773 | 1.3.99.— |
| SRS30216 | | | |
| Kineococcus radiotolerans | plsB(D311E) | ZP_00617279 | 2.3.1.15 |
| SRS30216 | | | |
| Kineococcus radiotolerans | aceE | ZP_00617600 | 1.2.4.1 |
| SRS30216 | | | |
| Kineococcus radiotolerans | aceF | ZP_00619307 | 2.3.1.12 |
| SRS30216 | | | |
| Kineococcus radiotolerans | fabH | ZP_00618003 | 2.3.1.180 |
| SRS30216 | | | |
| Kineococcus radiotolerans | fabD | ZP_00617602 | 2.3.1.39 |
| SRS30216 | | | |
| Kineococcus radiotolerans | fabG | ZP_00615651 | 1.1.1.100 |
| SRS30216 | | | |
| Kineococcus radiotolerans | acpP | ZP_00617604 | 3.1.26.3, |
| SRS30216 | | | 1.6.5.3, 1.6.99.3 |
| Kineococcus radiotolerans | fabF | ZP_00617605 | 2.3.1.179 |
| SRS30216 | | | |
| Kineococcus radiotolerans | gpsA | ZP_00618825 | 1.1.1.94 |
| SRS30216 | | | |
| Kineococcus radiotolerans | ldhA | ZP_00618879 | 1.1.1.28 |
| SRS30216 | | | |
| Rhodospirillum rubrum | accA | YP_425310 | 6.4.1.2 |
| Rhodospirillum rubrum | accB | YP_427521 | 6.4.1.2 |
| Rhodospirillum rubrum | accC | YP_427522/YP_425144/YP_427028/ | 6.3.4.14, 6.4.1.2 |
| | | YP_426209/ | |
| | | YP_427404 | |
| Rhodospirillum rubrum | accD | YP_428511 | 6.4.1.2 |
| Rhodospirillum rubrum | fadE | YP_427035 | 1.3.99.— |
| Rhodospirillum rubrum | aceE | YP_427492 | 1.2.4.1 |
| Rhodospirillum rubrum | aceF | YP_426966 | 2.3.1.12 |
| Rhodospirillum rubrum | fabH | YP_426754 | 2.3.1.180 |
| Rhodospirillum rubrum | fabD | YP_425507 | 2.3.1.39 |
| Rhodospirillum rubrum | fabG | YP_425508/YP_425365 | 1.1.1.100 |
| Rhodospirillum rubrum | acpP | YP_425509 | 3.1.26.3, |
| | | | 1.6.5.3, 1.6.99.3 |
| Rhodospirillum rubrum | fabF | YP_425510/YP_425510/ | 2.3.1.179 |
| | | YP_425285 | |
| Rhodospirillum rubrum | gpsA | YP_428652 | 1.1.1.94 |
| | | | 1.1.1.27 |
| Rhodospirillum rubrum | ldhA | YP_426902/YP_428871 | 1.1.1.28 |
| Vibrio furnissii | accA | 1, 16 | 6.4.1.2 |
| Vibrio furnissii | accB | 2, 17 | 6.4.1.2 |
| Vibrio furnissii | accC | 3, 18 | 6.3.4.14, 6.4.1.2 |
| Vibrio furnissii | accD | 4, 19 | 6.4.1.2 |
| Vibrio furnissii | fadE | 5, 20 | 1.3.99.— |
| Vibrio furnissii | plsB(D311E) | 6, 21 | 2.3.1.15 |
| Vibrio furnissii | aceE | 7, 22 | 1.2.4.1 |
| Vibrio furnissii | aceF | 8, 23 | 2.3.1.12 |
| Vibrio furnissii | fabH | 9, 24 | 2.3.1.180 |
| Vibrio furnissii | fabD | 10, 25 | 2.3.1.39 |
| Vibrio furnissii | fabG | 11, 26 | 1.1.1.100 |
| Vibrio furnissii | acpP | 12, 27 | 3.1.26.3, |
| | | | 1.6.5.3, 1.6.99.3 |
| Vibrio furnissii | fabF | 13, 28 | 2.3.1.179 |
| Vibrio furnissii | gpsA | 14, 29 | 1.1.1.94 |
| Vibrio furnissii | ldhA | 15, 30 | 1.1.1.27, |
| | | | 1.1.1.28 |
| Stenotrophomonas maltophilia | accA | ZP_01643799 | 6.4.1.2 |
| R551-3 | | | |
| Stenotrophomonas maltophilia | accB | ZP_01644036 | 6.4.1.2 |
| R551-3 | | | |
| Stenotrophomonas maltophilia | accC | ZP_01644037 | 6.3.4.14, 6.4.1.2 |
| R551-3 | | | |
| Stenotrophomonas maltophilia | accD | ZP_01644801 | 6.4.1.2 |
| R551-3 | | | |
| Stenotrophomonas maltophilia | fadE | ZP_01645823 | 1.3.99.— |
| R551-3 | | | |
| Stenotrophomonas maltophilia | plsB(D311E) | ZP_01644152 | 2.3.1.15 |
| R551-3 | | | |
| Stenotrophomonas maltophilia | aceE | ZP_01644724 | 1.2.4.1 |
| R551-3 | | | |
| Stenotrophomonas maltophilia | aceF | ZP_01645795 | 2.3.1.12 |
| R551-3 | | | |
| Stenotrophomonas maltophilia | fabH | ZP_01643247 | 2.3.1.180 |
| R551-3 | | | |
| Stenotrophomonas maltophilia | fabD | ZP_01643535 | 2.3.1.39 |
| R551-3 | | | |
| Stenotrophomonas maltophilia | fabG | ZP_01643062 | 1.1.1.100 |
| R551-3 | | | |
| Stenotrophomonas maltophilia | acpP | ZP_01643063 | 3.1.26.3 |
| R551-3 | | | 1.6.5.3, 1.6.99.3 |
| Stenotrophomonas maltophilia | fabF | ZP_01643064 | 2.3.1.179 |
| R551-3 | | | |
| Stenotrophomonas maltophilia | gpsA | ZP_01643216 | 1.1.1.94 |
| R551-3 | | | |
| Stenotrophomonas maltophilia | ldhA | ZP_01645395 | 1.1.1.28 |
| R551-3 | | | |
| Synechocystis sp. PCC6803 | accA | NP_442942 | 6.4.1.2 |
| Synechocystis sp. PCC6803 | accB | NP_442182 | 6.4.1.2 |
| Synechocystis sp. PCC6803 | accC | NP_442228 | 6.3.4.14, |
| | | | 6.4.1.2 |
| Synechocystis sp. PCC6803 | accD | NP_442022 | 6.4.1.2 |
| Synechocystis sp. PCC6803 | fabD | NP_440589 | 2.3.1.39 |
| Synechocystis sp. PCC6803 | fabH | NP_441338 | 2.3.1.180 |
| Synechocystis sp. PCC6803 | fabF | NP_440631 | 2.3.1.179 |
| Synechocystis sp. PCC6803 | fabG | NP_440934 | 1.1.1.100, |
| | | | 3.1.26.3 |
| Synechocystis sp. PCC6803 | fabZ | NP_441227 | 4.2.1.60 |
| Synechocystis sp. PCC6803 | fabl | NP_440356 | 1.3.1.9 |
| Synechocystis sp. PCC6803 | acp | NP_440632 | |
| Synechocystis sp. PCC6803 | fadD | NP_440344 | 6.2.1.3 |
| Synechococcus elongates | accA | YP_400612 | 6.4.1.2 |
| PCC7942 | | | |
| Synechococcus elongates | accB | YP_401581 | 6.4.1.2 |
| PCC7942 | | | |
| Synechococcus elongates | accC | YP_400396 | 6.3.4.14, |
| PCC7942 | | | 6.4.1.2 |
| Synechococcus elongates | accD | YP_400973 | 6.4.1.2 |
| PCC7942 | | | |
| Synechococcus elongates | fabD | YP_400473 | 2.3.1.39 |
| PCC7942 | | | |
| Synechococcus elongates | fabH | YP_400472 | 2.3.1.180 |
| PCC7942 | | | |
| Synechococcus elongates | fabF | YP_399556 | 2.3.1.179 |
| PCC7942 | | | |
| Synechococcus elongates | fabG | YP_399703 | 1.1.1.100, |
| PCC7942 | | | 3.1.26.3 |
| Synechococcus elongates | fabZ | YP_399947 | 4.2.1.60 |
| PCC7942 | | | |
| Synechococcus elongates | fabl | YP_399145 | 1.3.1.9 |
| PCC7942 | | | |
| Synechococcus elongates | acp | YP_399555 | |
| PCC7942 | | | |
| Synechococcus elongates | fadD | YP_399935 | 6.2.1.3 |
| PCC7942 | | | |

In regard to the information in Table 13, Accession Numbers are from GenBank, Release 159.0 as of Apr. 15, 2007, EC Numbers are from KEGG, Release 42.0 as of April 2007 (plus daily updates up to and including May. 9, 2007), results for Erwinia amylovora strain Ea273 are taken from the Sanger sequencing center, completed shotgun sequence as of May 9, 2007, positions for Erwinia represent locations on the Sanger psuedo-chromosome, sequences from Vibrio furnisii M1 are from the LS9 VFM1 pseudochromosome, v2 build, as of Sep. 28, 2006, and include the entire gene, and may also include flanking sequence.

### Example 7

### Additional Exemplary Production Hosts

The present example provides additional alternative productions host.

Various production hosts, and specific gene combinations or manipulations are provided in Table 14. The specific combinations of genes added to the production hosts can be achieved using the methods described herein and the production hosts can be used as described in the examples above.

The various production hosts provide two biosynthetic pathways for producing fatty acids, fatty alcohols, and esters.

Production hosts 1 and 2 in Table 14 both produce fatty acids. Production host 1 can be used to produce fatty acids. Production host 1 is a production host cell that is engineered to have the synthetic enzymatic activities indicated by the “x” marks in the rows which identify the genes (see “x” identifying acetyl-CoA carboxylase, thio-esterase, and acyl-CoA synthase activity). Production host cells can be selected from bacteria, yeast, and fungi. These genes can also be transformed into a production host cell that is modified to contain one or more of the genetic manipulations described in Example 1. As provided in Table 14 additional production hosts can be created using the indicated exogenous genes.

TABLE 14
Combination of genes useful for making genetically engineered production hosts

| | | Genes/gene | Fatty acids | | Fatty esters | |
|---|---|---|---|---|---|---|
| | | accession | Prod. | Prod. | Prod. | Prod. |
| Peptide | Sources of genes | number | host 1 | host 2 | host 1 | host 2 |
| acetyl-CoA carboxylase | E. coli | accABCD | X | X | X | X |
| thio-esterase | E. coli | tesA | X | | X | X |
| | E. coli | tesB/ | | | | |
| | | NC_000913 | | | | |
| | Cinnamomum | ccFatB | | | | |
| | camphora | | | | | |
| | Umbellularia | umFatB | | X | | |
| | californica | | | | | |
| | Cuphea | chFatB2 | | | | |
| | hookeriana | | | | | |
| | Cuphea | chFatB3 | | | | |
| | hookeriana | | | | | |
| | Cuphea | chFatA | | | | |
| | hookerian | | | | | |
| | Arabidopsis | AtFatA1 | | | | |
| | thaliana | | | | | |
| | Arabidopsis | AtFatB1 | | | | |
| | thaliana | [M141T] | | | | |
| acyl-CoA synthase | E. coli | fadD | X | X | X | X |
| | Stenotrophomonas | fadD | | | | |
| | maltophilia R551-3 | homolog/ | | | | |
| | | ZP_01644857 | | | | |
| Ester synthase/alcohol | Fundibacter | WST9 | | | | |
| acyl-transferase | jadensis DSM | | | | | |
| | 12178 | | | | | |
| | Alcanivora | atfA1/ | | | | |
| | borkumensis | accession | | | | |
| | | NC_00826.1 | | | | |
| | Alcanivora | atfA2/ | | | | |
| | borkumensis | accession | | | | |
| | | NC_00826.1 | | | | |
| | Marinobacter- | WS1/ | | | | |
| | hydrocarbonoclasticus | (EF219276.1) | | | | |
| | Marinobacter- | WS2/ | | | | |
| | hydrocarbonoclasticus | EF219377.1 | | | | |
| | Acinetobacter | WSadp1 | | | | X |
| | baylyl ADP1 | | | | | |
| | Mus | mWS | | | | |
| | musculus | | | | | |
| | Homo | hWS | | | | |
| | sapiens | | | | | |
| | Fragaria × | SAAT | | | | |
| | ananassa | | | | | |
| | Malus × | MpAAT | | | | |
| | domestica | | | | | |
| | Simmondsia | JjWS | | | | |
| | chinensis | (AAD38041) | | | | |
| Transport protein | Acinetobacter | unknown | | | X | X |
| | sp. HO1-N | | | | | |

### Example 8

### Production

The present example describes one example for part of a production process.

Production hosts are engineered to express umuC and umuD from E. coli in pBAD24 under the prpBCDE promoter system through de novo synthesis of this gene with the appropriate end-product production genes. For small scale hydrocarbon product production, E. coli BL21(DE3) cells harbouring pBAD24 (with ampicillin resistance and the end-product synthesis pathway) as well as pUMVC1 (with kanamycin resistance and the acetyl CoA/malonyl CoA over-expression system) are incubated overnight at at 37° C. shaken at >200 rpm 2 L flasks in 500 ml LB medium supplemented with 75 micrograms/mL ampicillin and 50 micrograms/ml kanamycin until cultures reached an OD₆₀₀ of >0.8. Upon achieving an OD₆₀₀ of >0.8, cells are supplemented with 25 mM sodium proprionate (pH 8.0) to activate the engineered gene systems for production as well as to stop cellular proliferation (through activation of UmuC and UmuD proteins). Induction is performed for 6 hours at 30° C. After incubation, production media is examined for product using GC-MS (as described in the following example).

For large scale product production, the engineered microorganisms can be grown in 10 L, 100 L, 10×10⁵ L or larger batches and manipulated to express desired products based on the specific genes encoded in plasmids as appropriate.

E. coli BL21(DE3) cells harbouring pBAD24 (with ampicillin resistance and the end-product synthesis pathway) as well as pUMVC1 (with kanamycin resistance and the acetyl-CoA/malonyl-CoA over-expression system) are incubated from a 500 mL seed culture for 10 L fermentations (5 L for 100 L fermentations) in LB media (glycerol free) at 37° C. shaken at >200 rpm until cultures reached an OD600 of >0.8 (typically 16 hours) incubated with 50 micrograms/mL kanamycin and 75 micrograms/mL ampicillin. The production media is supplemented to maintain a 25 mM sodium proprionate (pH 8.0) to activate the engineered in gene systems for production as well as to stop cellular proliferation (through activation of umuC and umuD proteins). Media is continuously supplemented with glucose to maintain a concentration of 90 g/100 mL. After the first hour of induction, aliquots of no more than 10% of the total volume are removed each hour and allowed to sit unaggitated so as to allow the hydrocarbon product to rise to the surface and undergo a spontaneous phase separation. The hydrocarbon component is then collected and the aqueous phase returned to the reaction chamber. The reaction chamber is operated continuously. When the OD₆₀₀ drops below 0.6, the cells are replaced with a new batch grown from a seed culture.

While the above example outlines one embodiment for how the production process can occur, as will be appreciated by one of skill in the art, additional processing or refinement can occur to the product. In some embodiments, such as in fatty ester production, subsequent to isolation the fatty esters can be washed briefly in 1 M HCl to split the ester bond, and returned to pH 7 with extensive washing with distilled water. In some embodiments, the product can be purified to remove excess water. In some embodiments, fine solids can be removed that might affect injection nozzles or prefilters in engines. In some embodiments, the bioester can also be processed to remove species that have poor volatility and would lead to deposit formation. Traces of sulfur compounds that may be present can be removed. It will be appreciated that steps for removing substances from the product can include one or more of washing, adsorption, distillation, filtration, centrifugation, settling, or coalescence.

### Example 9

### Product Characterization

The present example outlines an embodiment for characterizing a product of a production host.

To characterize and quantify, fatty esters, gas chromatography (GC) coupled with electron impact mass spectra (MS) detection can be used. Fatty esters can be dissolved in an appropriate volatile solvent, such as ethyl acetate before GC-MS analysis.

The samples can be analyzed on a 30 m DP-5 capillary column using the following method. After a 1 μL splitless injection onto the GC/MS column, the oven can be held at 100° C. for 3 minutes. The temperature can be ramped up to 320° C. at a rate of 20° C./minute. The oven can be held at 320° C. for an additional 5 minutes. The flow rate of the carrier gas helium can be 1.3 mL/minute. The MS quadrapole can be scanned from 50 to 550 m/z. Retention times and fragmentation patterns of product peaks can be compared with authentic references to confirm peak identity.

Quantification can be carried out by injecting various concentrations of the appropriate authentic references using the GC/MS method described above. This information can be used to generate a standard curve with response (total integrated ion count) versus concentration.

### Example 10

### Mixed Alcohols in Fatty Ester Production

The present example demonstrates how a mixed fatty ester product (where the population of fatty esters include at least two different A groups) can be made via a mixed alcohol starting mixture. In addition, the present example demonstrates the ability of a production host to utilize alcohols other than ethanol to produce various fatty esters and to do so simultaneously.

M9 minimal media (6 g/L Na2HPO4, 3 g/L KH2PO4, 0.5 g/L NaCl, 1 g/L NH4Cl, 1 mg/L thiamine, 1 mM MgSO4, 0.1 mM CaCl2, 20 g/L glucose) productions were carried out using E. coli strain C41 (DE3 ΔfadE) carrying the plasmid pACYCop-adp1WS under transcriptional control of the trc promoter (pTrcHisA2 plasmid (Invitrogen)) as the production host. Cells were cultured using the standard M9 fermentation protocol. In brief, a single colony or a scraping from a frozen glycerol stock is used to inoculate an LB+appropriate antibiotics overnight pre-seed culture. Using a 1:100 dilution of the pre-seed culture, an LB+antibiotics seed culture is inoculated. The seed culture is allowed to grow at 37° C. with shaking until OD₆₀₀ is between 1.0 and 2.0. 2 mL of the seed culture is then used to inoculate a 20 mL M9 media culture in a 125 mL shake flask. These cultures were allowed to grow at 37° C. with shaking until the OD₆₀₀=1.0 at which point the cells are induced with IPTG at a final concentration of 1 mM and fed 2% final volume pure alcohol or a mixture of different alcohols. Fermentation was carried out for an additional 20 hours post-induction at the desired temperature before extraction with ethyl acetate for GC/MS analysis. For this example, cells were fed either 2% final total volume of ethanol, a mixture of three alcohols (equal parts methanol, ethanol, isopropanol), or a mixture of four alcohols (equal parts methanol, ethanol, isopropanol, and propanol) at induction. The production host was allowed to process the alcohol(s) and media contents for an additional 20 hours post-induction at 30° C. before extracting with ethyl acetate.

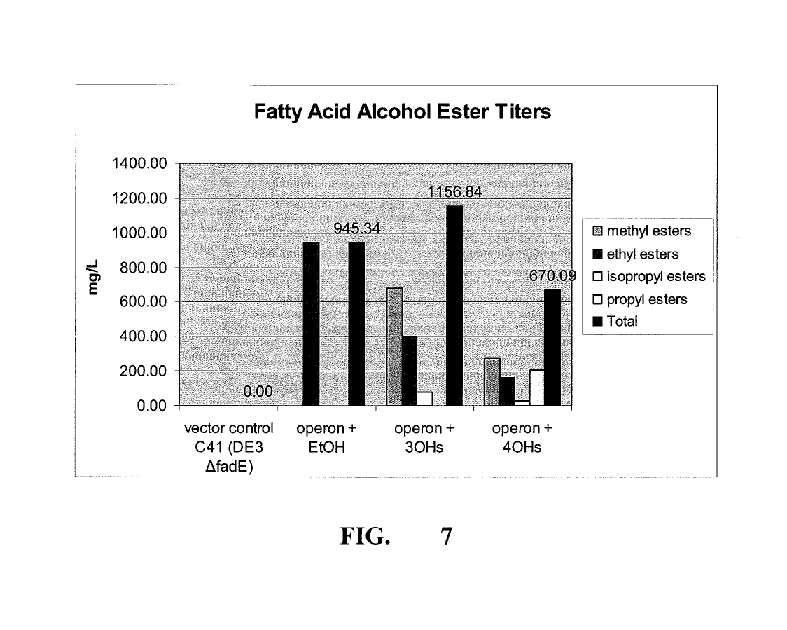

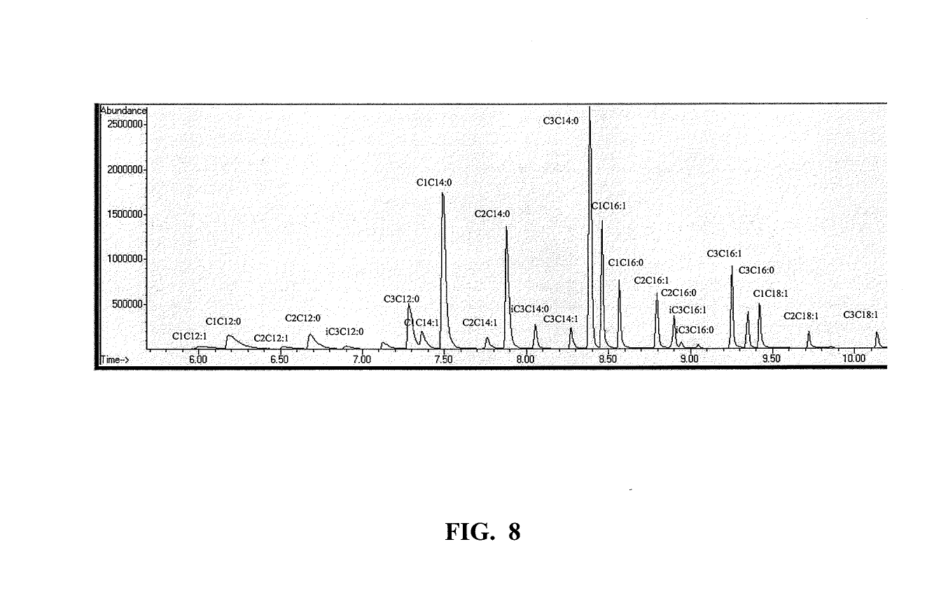

When analyzed by GC/MS, the fatty esters corresponding to all four alcohols tested could be identified. The total titer of cultures fed the 3-alcohol mixture (1156.84 mg/L) was higher than those fed only ethanol (945.34 mg/L). Cells fed all four alcohols had the lowest overall titer (670.09 mg/L). For both sets of cultures fed the alcohol mixtures, methyl esters were the most abundant fatty esters, followed by the ethyl esters or propyl esters. The results of the fatty esters produced are displayed in FIG. 7. The results of the GC/MS analysis are shown in FIG. 8 and in Tables 15-19.

TABLE 15

| | Strain | | | | | | | |
|---|---|---|---|---|---|---|---|---|
| | C1C12:1 | C1C12:0 | C2C12:1 | C2C12:0 | iC3C12:0 | C3C12:0 | C1C14:1 | C1C14:0 |
| vector | 0.00 | 0.00 | 0.00 | 0.00 | 0.00 | 0.00 | 0.00 | 0.00 |
| control | | | | | | | | |
| ethanol fed | 0.00 | 0.00 | 42.75 | 156.95 | 0.00 | 0.00 | 0.00 | 0.00 |
| methanol, | 25.15 | 100.96 | 18.49 | 66.38 | 15.32 | 0.00 | 53.44 | 249.42 |
| ethanol, | | | | | | | | |
| isopropanol | | | | | | | | |
| fed | | | | | | | | |
| methanol, | 8.28 | 30.54 | 5.72 | 24.36 | 5.26 | 41.27 | 19.90 | 106.33 |
| ethanol, | | | | | | | | |
| isopropanol, | | | | | | | | |
| propanol fed | | | | | | | | |

TABLE 16

| | Strain | | | | | | | |
|---|---|---|---|---|---|---|---|---|
| | C2C14:1 | C2C14:0 | iC3C14:0 | C3C14:0 | C1C16:1 | C1C16:0 | C2C16:1 | C2C16:0 |
| vector | 0.00 | 0.00 | 0.00 | 0.00 | 0.00 | 0.00 | 0.00 | 0.00 |
| control | | | | | | | | |
| ethanol fed | 74.13 | 355.10 | 0.00 | 0.00 | 0.00 | 0.00 | 172.46 | 84.11 |
| methanol, | 31.65 | 161.96 | 43.20 | 0.00 | 141.10 | 63.50 | 67.68 | 31.11 |
| ethanol, | | | | | | | | |
| isopropanol | | | | | | | | |
| fed | | | | | | | | |
| methanol, | 10.46 | 71.27 | 15.69 | 101.38 | 55.18 | 31.06 | 25.96 | 16.31 |
| ethanol, | | | | | | | | |
| isopropanol, | | | | | | | | |
| propanol fed | | | | | | | | |

TABLE 17

| | Strain | | | | | | | Total |
|---|---|---|---|---|---|---|---|---|
| | iC3C16:1 | iC3C16:0 | C3C16:1 | C3C16:0 | C1C18:1 | C2C18:1 | C3C18:1 | (mg/L) |
| vector | 0.00 | 0.00 | 0.00 | 0.00 | 0.00 | 0.00 | 0.00 | 0.00 |
| control | | | | | | | | |
| ethanol fed | 0.00 | 0.00 | 0.00 | 0.00 | 0.00 | 59.84 | 0.00 | 945.34 |
| methanol, | 12.92 | 6.30 | 0.00 | 0.00 | 47.94 | 20.32 | 0.00 | 1156.84 |
| ethanol, | | | | | | | | |
| isopropanol | | | | | | | | |
| fed | | | | | | | | |
| methanol, | 4.20 | 2.45 | 35.58 | 18.27 | 22.47 | 9.36 | 8.78 | 670.09 |
| ethanol, | | | | | | | | |
| isopropanol, | | | | | | | | |
| propanol fed | | | | | | | | |

TABLE 18

| | Final | |
|---|---|---|
| Strain | OD₆₀₀ | Total/OD |
| vector | 2.98 | 0.00 |
| control | | |
| ethanol fed | 4.57 | 206.86 |
| methanol, | 4.79 | 241.34 |
| ethanol, | | |
| isopropanol | | |
| fed | | |
| methanol, | 3.60 | 185.97 |
| ethanol, | | |
| isopropanol, | | |
| propanol fed | | |

As will be appreciated by one of skill in the art, the results noted in Example 10 indicate that using a mixture of alcohols can boost the overall fatty ester titer over using ethanol alone (see 1156.84 mg/L of total (methanol, ethanol, and isopropyl) in FIG. 7 compared to 945.34 mg/L total (ethanol alone)). In addition, the sum of the fatty acid methyl ester (FAME) and fatty acid ethyl ester (FAEE) titers was higher than the total FAEE titer for cells fed ethanol only (1079.11 mg/L vs 945.34 mg/L). Thus, it is apparent that the addition of methanol to a fatty ester production process can result in a synergistic and unexpected increase in the output of fatty esters.

### Example 11

### Mixed Alcohols in Fatty Ester Production

The present example further examines the characteristics of fatty ester products resulting from using two starting alcohols (ethanol and methanol).

The experiments were carried out using strain MG1655 (ΔfadE) carrying the plasmid pCLop-atfA1. Cells were cultured in Hu-9, a minimal media based on M9 supplemented with uracil (20 ug/mL) and trace minerals (27 mg/L FeCl3-6H2O, 2 mg/L ZnCl-4H2O, 2 mg/L CaCl2-6H2O, 2 mg/L Na2MoO4-2H2O, 1.9 mg/L CuSO4-5H2O, 0.5 mg/L H3BO3, 100 mL/L concentrated HCl). The standard M9 fermentation protocol was followed.

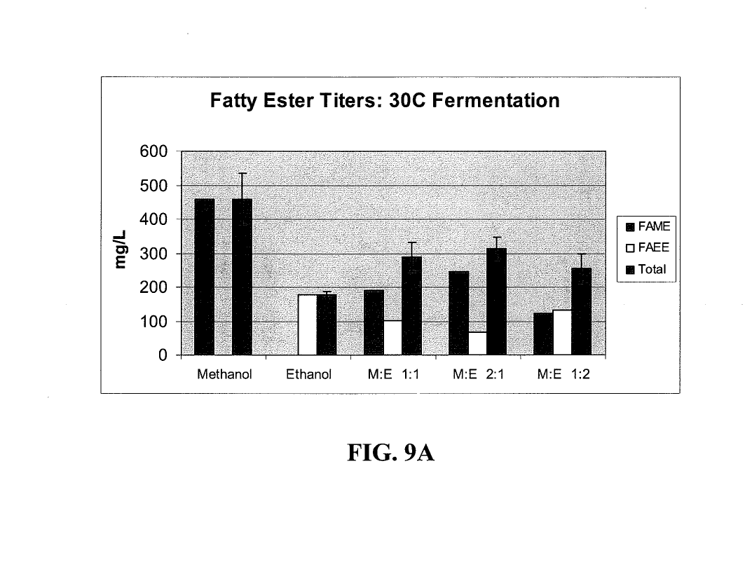

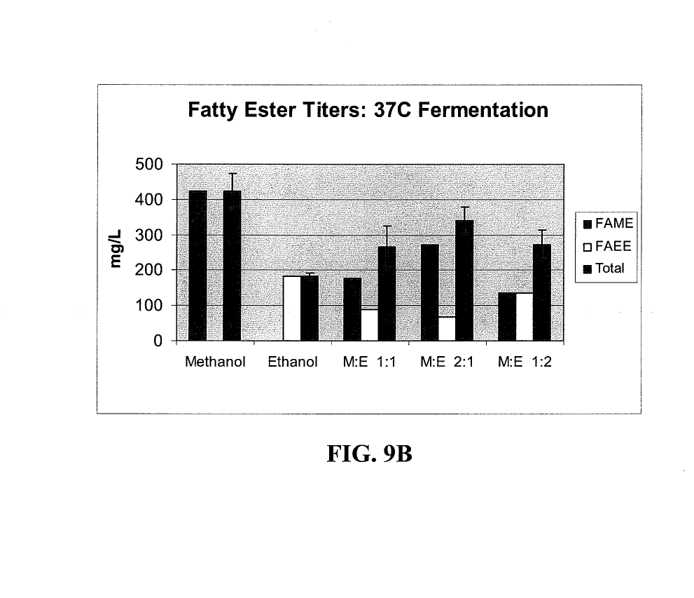

At induction, cells were fed a 2% total final volume of methanol alone, ethanol alone, or a mixture of the two in different ratios. The fatty ester production host was allowed to process the alcohol mixture for an additional 20 hours as above. Two different process temperatures were examined either 30° C. or 37° C. The fatty ester products were analyzed via GC-MS and the results are shown in FIGS. 9A and 9B and in Table 19.

The GC/MS data show that feeding methanol alone produced the highest overall titer (460 mg/L and 424 mg/L for the 30° C. and 37° C., respectively) while ethanol alone the lowest (178 mg/L and 183 mg/L). Feeding a mixture of the two alcohols resulted in titers falling between the fatty ester titers observed for the single alcohol feedings. At both fermentation temperatures, cells fed alcohol mixtures having either more methanol than ethanol or equal parts of both produced more FAMEs than FAEEs. Only when cells were fed a higher ratio of ethanol did they produce roughly equal parts FAMEs and FAEEs.

TABLE 19

| | |
|---|---|
| 30° C. | 37° C. |

| | Total | | Total | |
|---|---|---|---|---|
| | Titer | % vs | Titer | % vs |
| | (mg/L) | EtOH | (mg/L) | EtOH |
| Methanol | 459.95 | 258% | 424.28 | 231% |
| Ethanol | 178.28 | 100% | 183.42 | 100% |
| M:E 1:1 | 289.51 | 162% | 267.26 | 146% |
| M:E 2:1 | 314.50 | 176% | 341.57 | 186% |
| M:E 1:2 | 255.78 | 143% | 271.21 | 148% |

The data in Table 19 show the total titers of methyl and ethyl esters for the 30° C. and 37° C. fermentations. Table 19 also displays the percent ratios of total fatty esters when compared to the total titer produced by cells fed ethanol only.

Additional data regarding the results is presented in Tables 20-23 (with Tables 20 and 21 showing the results from 30° C. process and Tables 22 and 23 showing the results from the 37° C. process):

TABLE 20

| | C1C11 | C1C12 | C2C12 | C1C13 | C2C13 | C1C14:1 | C1C14:0 | C2C14:1 | C2C14:0 | C1C15 | C2C15 | C1C16:1 |
|---|---|---|---|---|---|---|---|---|---|---|---|---|
| Methanol | 11.35 | 66.29 | 0.00 | 4.17 | 0.00 | 18.26 | 212.32 | 0.00 | 0.00 | 5.25 | 0.00 | 58.52 |
| Ethanol | 0.00 | 0.00 | 34.83 | 0.00 | 1.33 | 0.00 | 0.00 | 7.50 | 82.35 | 0.00 | 1.95 | 0.00 |
| M:E 1:1 | 2.64 | 33.80 | 17.96 | 0.74 | 1.12 | 6.23 | 88.18 | 4.69 | 44.37 | 1.06 | 1.85 | 21.60 |
| M:E 2:1 | 3.45 | 40.42 | 11.30 | 0.97 | 0.89 | 25.04 | 103.61 | 3.48 | 29.12 | 1.35 | 1.52 | 27.71 |
| M:E 1:2 | 1.81 | 21.55 | 23.62 | 0.28 | 1.34 | 4.09 | 58.05 | 5.71 | 59.27 | 0.68 | 2.22 | 14.65 |

TABLE 21

| | | | | | | | | | | Ratio of total FAEE |
|---|---|---|---|---|---|---|---|---|---|---|
| | | | | | | | | | | with respect to titers |
| | | | | | | | | | | from ethanol |
| | C1C16:0 | C2C16:1 | C2C16:0 | C1C18:1 | C2C18:1 | C2C18:0 | Total | Total FAME | Total FAEE | feeding alone |
| Methanol | 46.42 | 0.00 | 0.00 | 37.38 | 0.00 | 0.00 | 459.95 | 459.95 | 0.00 | 2.58 |
| Ethanol | 0.00 | 18.35 | 16.96 | 0.00 | 14.04 | 0.97 | 178.28 | 0.00 | 178.28 | 1.00 |
| M:E 1:1 | 19.79 | 10.88 | 10.03 | 15.44 | 8.12 | 0.99 | 289.51 | 189.48 | 100.03 | 1.62 |
| M:E 2:1 | 25.19 | 7.72 | 6.83 | 19.42 | 5.58 | 0.91 | 314.50 | 247.16 | 67.34 | 1.76 |
| M:E 1:2 | 12.49 | 14.63 | 12.90 | 10.41 | 11.13 | 0.94 | 255.78 | 124.01 | 131.77 | 1.43 |

TABLE 22

| | C1C11 | C1C12 | C2C12 | C1C13 | C2C13 | C1C14:1 | C1C14:0 | C2C14:1 | C2C14:0 | C1C15 | C2C15 | C1C16:1 |
|---|---|---|---|---|---|---|---|---|---|---|---|---|
| Methanol | 4.81 | 46.02 | 0.00 | 4.02 | 0.00 | 9.01 | 220.48 | 0.00 | 0.00 | 6.03 | 0.00 | 34.87 |
| Ethanol | 0.00 | 0.00 | 30.47 | 0.00 | 0.75 | 0.00 | 0.00 | 4.70 | 94.64 | 0.00 | 1.11 | 0.00 |
| M:E 1:1 | 3.03 | 23.77 | 12.84 | 1.65 | 0.52 | 4.35 | 87.59 | 2.85 | 45.28 | 2.14 | 0.92 | 14.35 |
| M:E 2:1 | 3.85 | 34.65 | 9.39 | 2.21 | 0.51 | 6.11 | 139.10 | 2.42 | 34.50 | 2.90 | 0.99 | 21.58 |
| M:E 1:2 | 2.89 | 18.99 | 20.68 | 1.55 | 0.67 | 3.66 | 65.75 | 3.88 | 68.84 | 2.14 | 1.30 | 10.69 |

TABLE 23

| | | | | | | | | | % vs |
|---|---|---|---|---|---|---|---|---|---|
| | C1C16:0 | C2C16:1 | C2C16:0 | C1C18:1 | C2C18:1 | Total | FAME | FAEE | EtOH |
| Methanol | 63.25 | 0.00 | 0.00 | 35.79 | 0.00 | 424.28 | 424.28 | 0.00 | 2.31 |
| Ethanol | 0.00 | 13.80 | 22.97 | 0.00 | 14.97 | 183.42 | 0.00 | 183.42 | 1.00 |
| M:E 1:1 | 25.17 | 7.39 | 12.51 | 15.19 | 7.69 | 267.26 | 177.24 | 90.02 | 1.46 |
| M:E 2:1 | 38.76 | 5.64 | 9.62 | 23.29 | 6.05 | 341.57 | 272.45 | 69.12 | 1.86 |
| M:E 1:2 | 18.24 | 10.47 | 18.12 | 11.81 | 11.55 | 271.21 | 135.71 | 135.50 | 1.48 |

As demonstrated in Examples 10 and 11 above, in some embodiments, using an alcohol mixture containing methanol can be preferable to pure ethanol for the production of fatty esters, especially for fatty esters for biodiesel. For both ester synthases (WSadp1 and AtfA1) tested, methanol appeared to be the preferred substrate over ethanol, as indicated by the higher titers of FAMES vs FAEEs. Moreover, feeding methanol mixed with ethanol resulted in an increase in total fatty ester production by both strains tested.

### Example 12

### Methanol Biases the Fatty Ester Product to Longer B Sides

The present example demonstrates that the use of methanol in alcohol mixtures for the production of fatty esters can bias the fatty ester products in favor of longer B sides. The product from the 30° C. experiment noted in Example 11 was examined for the types of acyl chains (B sides) present in the fatty ester due to the use of a mixture of starting alcohols.

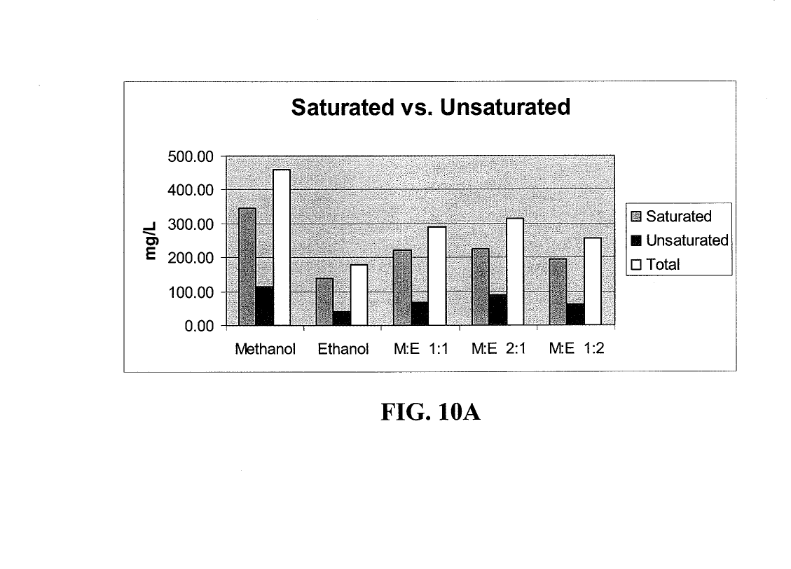

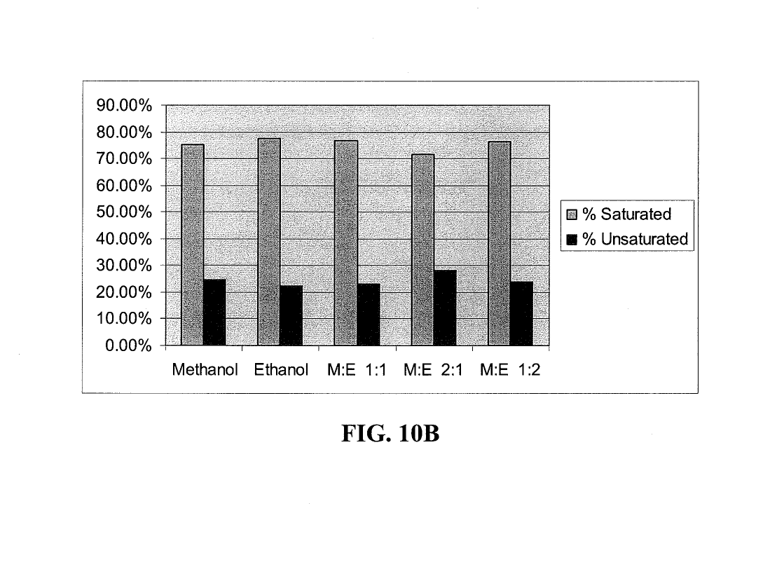

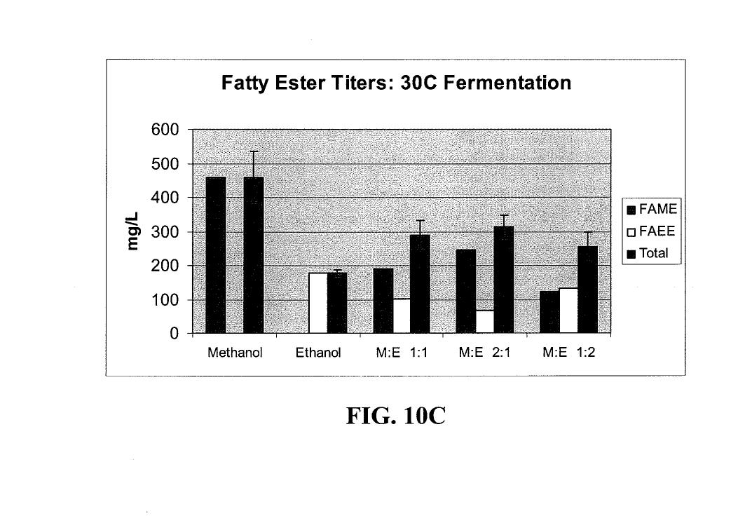

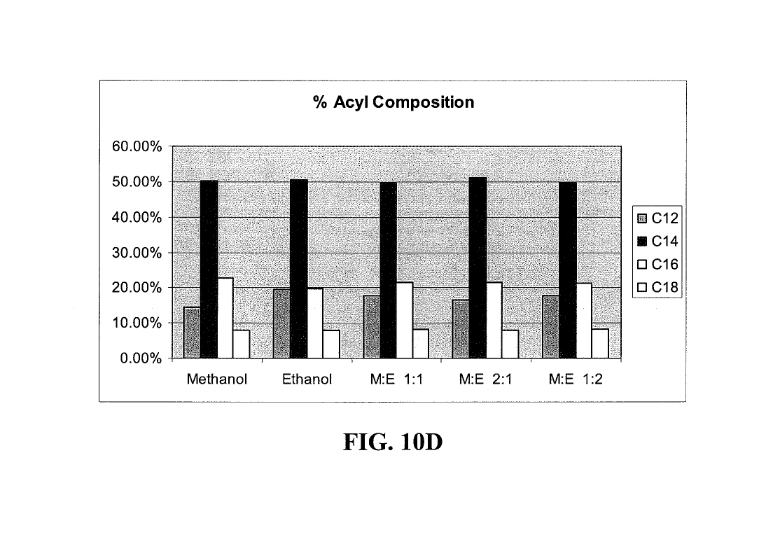

The results are presented in FIGS. 10A-10D and Tables 24-27. As can be seen in the data in the tables and FIGS. 10A-10D, the presence of methanol appears to bias the resulting product towards longer chain fatty esters (e.g., there is more C16), while the presence of ethanol results in higher levels of shorter chain fatty esters (more C12).

TABLE 24

| | C1C12:0 | C2C12:0 | C1C14:1 | C1C14:0 | C2C14:1 | C2C14:0 |
|---|---|---|---|---|---|---|
| Methanol | 14.41% | 0.00% | 3.97% | 46.16% | 0.00% | 0.00% |
| Ethanol | 0.00% | 19.54% | 0.00% | 0.00% | 4.21% | 46.19% |
| M:E 1:1 | 11.68% | 6.20% | 2.15% | 30.46% | 1.62% | 15.33% |
| M:E 2:1 | 12.85% | 3.59% | 7.96% | 32.94% | 1.11% | 9.26% |
| M:E 1:2 | 8.43% | 9.23% | 1.60% | 22.69% | 2.23% | 23.17% |

TABLE 25

| | C2C14:0 | C1C16:1 | C1C16:0 | C2C16:1 | C2C16:0 | C1C18:1 | C2C18:1 |
|---|---|---|---|---|---|---|---|
| Methanol | 0.00% | 12.72% | 10.09% | 0.00% | 0.00% | 8.13% | 0.00% |
| Ethanol | 46.19% | 0.00% | 0.00% | 10.29% | 9.51% | 0.00% | 7.87% |
| M:E 1:1 | 15.33% | 7.46% | 6.84% | 3.76% | 3.47% | 5.33% | 2.80% |
| M:E 2:1 | 9.26% | 8.81% | 8.01% | 2.45% | 2.17% | 6.18% | 1.77% |
| M:E 1:2 | 23.17% | 5.73% | 4.88% | 5.72% | 5.04% | 4.07% | 4.35% |

TABLE 26

| | C12 | C14 | C16 | C18 |
|---|---|---|---|---|
| Methanol | 14.41% | 50.13% | 22.82% | 8.13% |
| Ethanol | 19.54% | 50.40% | 19.81% | 7.87% |
| M:E 1:1 | 17.88% | 49.56% | 21.52% | 8.14% |
| M:E 2:1 | 16.45% | 51.27% | 21.45% | 7.95% |
| M:E 1:2 | 17.66% | 49.70% | 21.37% | 8.42% |

TABLE 27

| | % Saturated | % Unsaturated |
|---|---|---|
| Methanol | 75.18% | 24.82% |
| Ethanol | 77.63% | 22.37% |
| M:E 1:1 | 76.87% | 23.13% |
| M:E 2:1 | 71.72% | 28.28% |
| M:E 1:2 | 76.30% | 23.70% |

### Example 13

### Methanol Biases the Fatty Ester Product to Longer B Sides

The present example demonstrates that the use of methanol in alcohol mixtures for the production of fatty esters can bias the fatty ester products in favor of longer B sides. The product from the 37° C. experiment noted in Example 11 was examined for the types of B chain products that were produced.

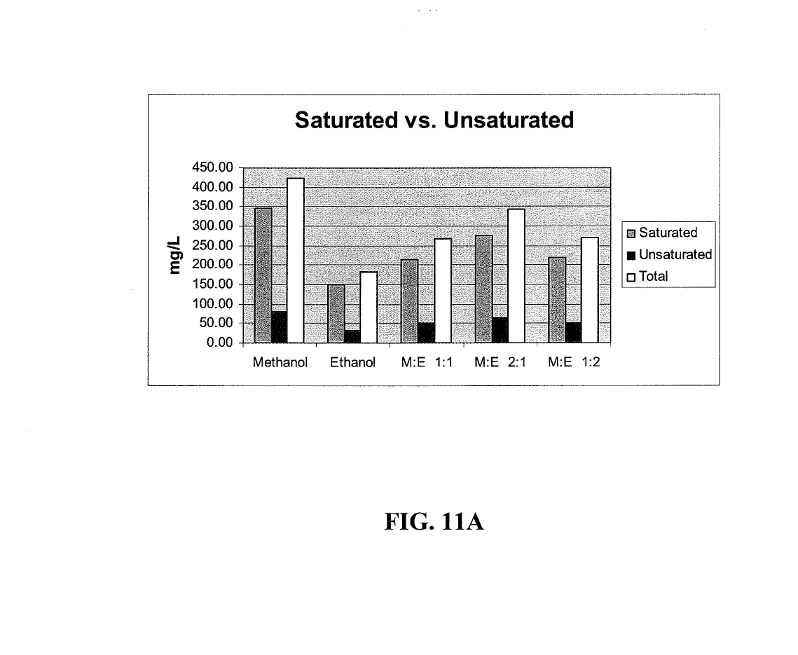

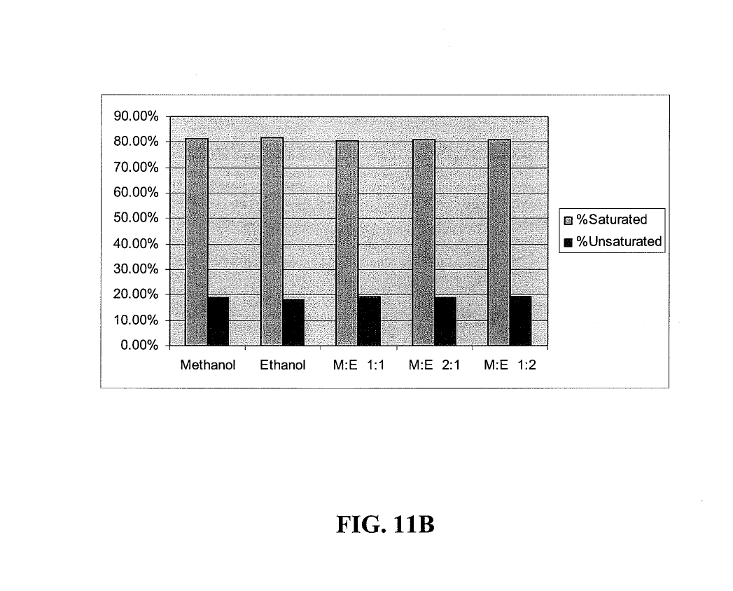

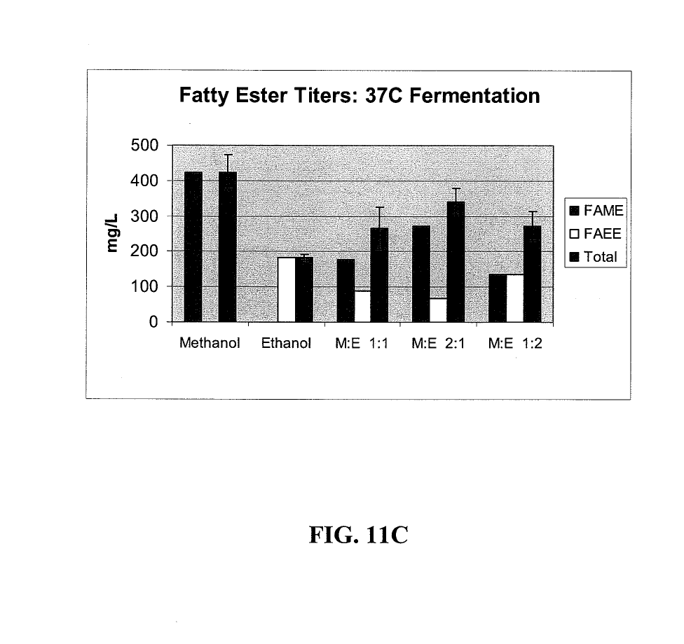

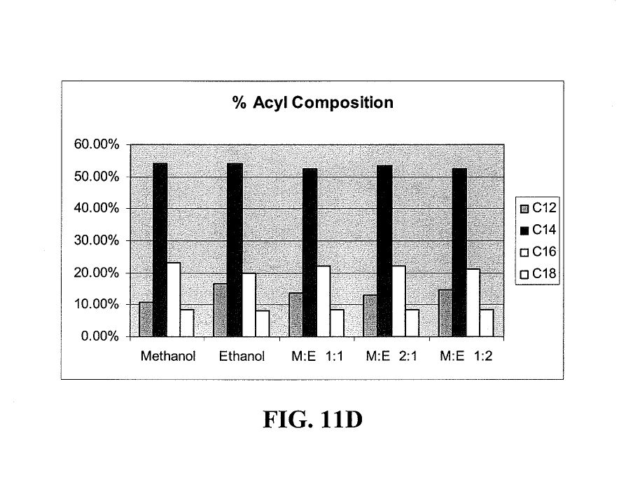

The results are presented in FIGS. 11A-11D and Tables 28-31. As can be seen in the data in the tables and FIGS. 11A-11D, the presence of methanol appears to bias the resulting product towards longer chain fatty esters (e.g., there is more C16), while the presence of ethanol results in higher levels of shorter chain fatty esters (more C12).

TABLE 28

| | C1C12:0 | C2C12:0 | C1C14:1 | C1C14:0 | C2C14:1 | C2C14:0 |
|---|---|---|---|---|---|---|
| Methanol | 10.85% | 0.00% | 2.12% | 51.97% | 0.00% | 0.00% |
| Ethanol | 0.00% | 16.61% | 0.00% | 0.00% | 2.56% | 51.60% |
| M:E 1:1 | 8.90% | 4.80% | 1.63% | 32.77% | 1.07% | 16.94% |
| M:E 2:1 | 10.14% | 2.75% | 1.79% | 40.72% | 0.71% | 10.10% |
| M:E 1:2 | 7.00% | 7.63% | 1.35% | 24.24% | 1.43% | 25.38% |

TABLE 29

| | C1C16:1 | C1C16:0 | C2C16:1 | C2C16:0 | C1C18:1 | C2C18:1 |
|---|---|---|---|---|---|---|
| Methanol | 8.22% | 14.91% | 0.00% | 0.00% | 8.43% | 0.00% |
| Ethanol | 0.00% | 0.00% | 7.52% | 12.52% | 0.00% | 8.16% |
| M:E 1:1 | 5.37% | 9.42% | 2.77% | 4.68% | 5.68% | 2.88% |
| M:E 2:1 | 6.32% | 11.35% | 1.65% | 2.82% | 6.82% | 1.77% |
| M:E 1:2 | 3.94% | 6.73% | 3.86% | 6.68% | 4.35% | 4.26% |

TABLE 30

| | C12 | C14 | C16 | C18 |
|---|---|---|---|---|
| Methanol | 10.85% | 54.09% | 23.13% | 8.43% |
| Ethanol | 16.61% | 54.16% | 20.05% | 8.16% |
| M:E 1:1 | 13.70% | 52.41% | 22.24% | 8.56% |
| M:E 2:1 | 12.89% | 53.32% | 22.13% | 8.59% |
| M:E 1:2 | 14.63% | 52.41% | 21.21% | 8.61% |

TABLE 31

| | % Saturated | % Unsaturated |
|---|---|---|
| Methanol | 81.22% | 18.78% |
| Ethanol | 81.75% | 18.25% |
| M:E 1:1 | 80.61% | 19.39% |
| M:E 2:1 | 80.95% | 19.05% |
| M:E 1:2 | 80.81% | 19.19% |

In light of Examples 12 and 13 described above and the results presented therein, it is clear that selecting a starting selection of alcohols can do more than allow one to obtain a desired population of A sides in a fatty ester population. In particular, it is clear that the length of the B side in a product is biased by starting with a specific alcohol or mixture of alcohols. Thus, in some embodiments, the desired B sides in a fatty ester composition can be biased or created by adding an appropriate amount of ethanol, methanol, or ethanol and methanol to the fatty ester production process. As noted above, increasing the amount of methanol in an alcohol mixture can decrease the concentration of shorter B sides (e.g., C12) and increase the bias to longer B sides (e.g., C16) while increasing the amount of ethanol in an alcohol mixture increases the shorter B sides (C12) and lowers the amount of the longer B sides (C16), relative to the products formed using alcohol mixtures without the increased amounts of methanol or ethanol.

In addition, Examples 12 and 13 also demonstrate that lower temperatures (30° C. vs. 37° C.) can be used to increase the amount of C12 and C14 in a produced fatty ester composition. In addition, this bias in favor of C12 at lower temperatures is additive to that observed due to the use of ethanol.

### Example 14

### Impact of Multiple Alcohols on Fatty Ester Saturation

The product produced in Example 10 was examined to determine how the mixture of multiple alcohols impacts the saturation of the B sides in a fatty ester product.

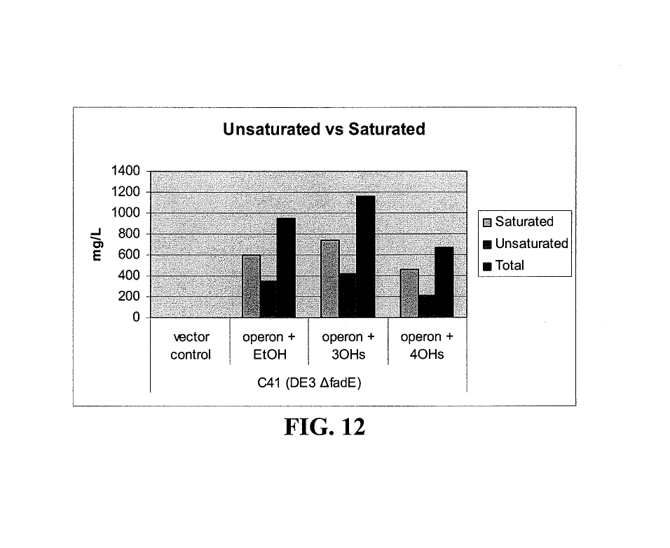

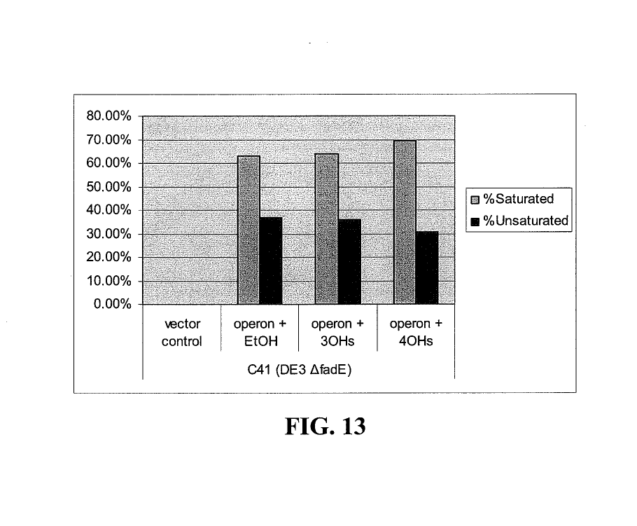

The results are presented in FIGS. 12 and 13 and Tables 32-34.

TABLE 32

| | | methyl | ethyl | isopropyl | propyl | |
|---|---|---|---|---|---|---|
| | | esters | esters | esters | esters | Total |
| C41 (DE3 ΔfadE) | vector control | 0.00 | 0.00 | 0.00 | 0.00 | 0.00 |
| | operon + EtOH | 0.00 | 945.34 | 0.00 | 0.00 | 945.34 |
| | operon + 3OHs | 681.52 | 397.59 | 77.74 | 0.00 | 1156.84 |
| | operon + 4OHs | 273.78 | 163.44 | 27.60 | 205.28 | 670.09 |

TABLE 33

| | | Saturated | Unsaturated | Total |
|---|---|---|---|---|
| C41 | vector control | 0 | 0 | 0 |
| (DE3, ΔfadE) | operon + EtOH | 596.1665 | 349.1751 | 945.3416 |
| | operon + 3OHs | 738.1544 | 418.6897 | 1156.844 |
| | operon + 4OHs | 464.1904 | 205.9036 | 670.094 |

TABLE 34

| | | % Saturated | % Unsaturated |
|---|---|---|---|
| C41 (DE3, ΔfadE) | vector control | 0.00% | 0.00% |
| | operon + EtOH | 63.06% | 36.94% |
| | operon + 3OHs | 63.81% | 36.19% |
| | operon + 4OHs | 69.27% | 30.73% |

Interestingly, the results suggest that increasing the variety of alcohols increases the saturation of the B sides in the fatty acid composition. This is especially interesting given the results in the previous examples, suggesting that the greater amount of ethanol present will result in great amounts of saturated fatty esters.

### Example 15

The present example demonstrates how one can select a specific fatty ester composition for production by selecting the appropriate alcohol.

One first selects a combination of fatty esters that are desired to be produced. In particular, one identifies which A sides should be present in the fatty esters of the final composition. When methyl and ethyl A sides are desired, one adds ethanol and methanol into the fatty ester production vessel along with the production substrate and the production host (e.g., an E. coli bacterium comprising a nucleic acid sequence encoding a thioesterase (EC 3.1.2.14), a wax synthase (EC 2.3.1.75), and an acyl-CoA synthetase (E.C.2.3.1.86), and having an attenuated acyl-CoA dehydrogenase gene). The fatty esters produced will have A sides that correspond to the length of the carbons in the provided alcohols. Thus, the fatty ester composition will include fatty ethyl esters and fatty methyl esters. In other embodiments, longer alcohols (e.g., propanol and/or isopropanol) can be provided to form products having longer A sides (e.g., fatty propyl esters and fatty isopropyl esters).

### Example 16

The present example demonstrates one method of producing a variety of alcohols for subsequent mixed fatty ester synthesis.

A mixed alcohol composition is produced in an alcohol production vessel using an alcohol production host, for example, Clostridium. The Clostridium will convert sugar into a variety of alcohols. Once the alcohols are produced, which can include butanol and ethanol, or butanol and isopropanol, or isopropanol, or ethanol, one or more to the alcohols is transported to a fatty ester production vessel where at least two alcohols will then be present.

The alcohols will be combined with a fatty ester production host and a fatty ester substrate. The fatty ester production host will create a mixture of fatty esters based upon the mixture of alcohols present in the fatty ester production vessel.

### Example 17

### Production of Biodiesel

The present example outlines how the fatty ester products can be further manipulated for use as a biodiesel.

The fatty ester product from any of the above fatty ester producing examples can be collected as outlined in Example 8. Once the hydrophobic phase is collected, the fatty esters can be further purified and concentrated if desired. In addition, various specific types of fatty esters can be isolated or concentrated as desired. The collected fatty ester composition can then be isolated by distillation to at least 90% fatty esters. In some cases, the collected fatty ester composition can be purified to be at least about 99% fatty esters. The concentrated product can then be used as a biodiesel fuel product for various biodiesel engines, e.g., as the combustible fuel in combustion engines in vehicles.

### Example 18

### Fuel Customization

The present example demonstrates how one can customize a biodiesel fuel that comprises at least two different fatty esters for various environments.

One identifies an environment in which the biodiesel is to be used. One identifies specific environmental aspects associated with the specific environment, for example, environmental temperature and air pressure. One then matches the desired type of fatty ester mixture (which will comprise at least two different fatty esters) for the specific environmental aspects (so that the desired fuel characteristics are exhibited in the identified environment) Once one identifies a desired fatty ester mixture, one prepares the desired fatty ester mixture via a mixture of at least two different alcohols, a production substrate, and a production host. The mixture of alcohols employed will be selected based upon the desired final composition of fatty esters. As noted above, the length of the A side, the B side, and the degree of saturation of the B side can all be influenced in a predictable manner via the use of specific initial alcohols, as disclosed herein.

Thus, one can customize biodiesel fuels to have a specific fatty ester composition via the manipulation of the initial alcohols used in the fatty ester production process.

### Example 19

### Production of Fatty Esters from Different Alcohols

The present example demonstrates a method for employing a single production host for the production of fatty acid methyl, ethyl, propyl, and isopropyl esters. The experiment involved the use of different alcohols in order to obtain the desired A side of the fatty ester.

Strains, Plasmids and Cultivation Condition

E. coli C41 (DE3) purchased from Lucigen (Middletown, Wis.) was used as the primary host for production of fatty esters. E. coli Top 10 (Invitrogen, Carlsbad, Calif.) was used for manipulation and propagation of plasmids. The antibiotic used to maintain the plasmid in E. coli strains was kanamycin (50 mg/L, final concentration). The ester synthase gene (atfA) from A. baylyi ADP1 was amplified with primer adp1ws_NdeI (5′-TCATATGGCGCCCATTACATCCG) and adp1ws_AvrII (5′-TCCTAGGAGGGCTAATTTAGCCCTTTAGTT). After amplification, the PCR product was digested with NdeI_and AvrII (underlined sites) and ligated with pCOLADuet-1 cut with NdeI and AvrII to produce pHZ1.43.

To evaluate fatty ester production, a starter culture of LB medium containing the appropriate antibiotics was inoculated from a single colony and grown over night at 37° C. This was used as an inoculum (1% v/v) for 50 ml of LB medium supplemented with the appropriate antibiotics. When the cell density of the culture reached OD₆₀₀ of 0.5, IPTG (1 mM) and methanol, or ethanol, or propanol, isopropanol or butanol or isobutanol (1% v/v) and potassium palmitate (0.1% W/V, final concentration) were added. 3 ml of each culture was then dispensed to three 16 ml glass tubes. These cultures were grown at 37° C. for 24 hours to allow for the production of the fatty esters.

Analysis of Fatty Esters

For quantification of total fatty esters, 750 ul of culture broth was collected. The cells were separated from spent medium via centrifugation at 12,000 RPM. The cells were resuspended with 750 ul of fresh LB medium. To the cell portion and the spent medium portion, 750 ul of ethyl acetate were added and then the mixtures were vortexed at top speed for 2 minutes. After phase separation by centrifugation at 3000 rpm for 2 minutes, the organic phase was withdrawn and directly analyzed by gas chromatography/mass spectrometry (GC/MS).

GC/MS analysis was performed on an Agilent 6580 (series II) equipped with a 30 m DP-5 capillary column. Each sample (1 uL) was analyzed with splitless injection. The temperature of the GC oven was held at 100° C. for 3 minutes and then increased to 320° C. at a rate of 20° C. per minutes. The oven was held at 320° C. for an additional 5 minutes. The flow rate of the helium carrier gas was 1.3 mL/minute. The MS quadrapole scans from 50 to 550 m/z. Commercial pure ethyl palmitate (# P9009 from Sigma) was used as the standard to quantify various fatty esters. The following authentic fatty esters, ethyl octanoate, ethyl decanoate, ethyl dodecanoate, ethyl myristate, ethyl palmitate, ethyl palmitoleate were used to identify corresponding compounds. Authentic ethyl oleate was used as a reference for the identification of ethyl cis-vaccenate. Fatty acid methyl esters, isopropyl esters and propyl esters produced from recombinant E. coli strains were determined in a similar fashion.

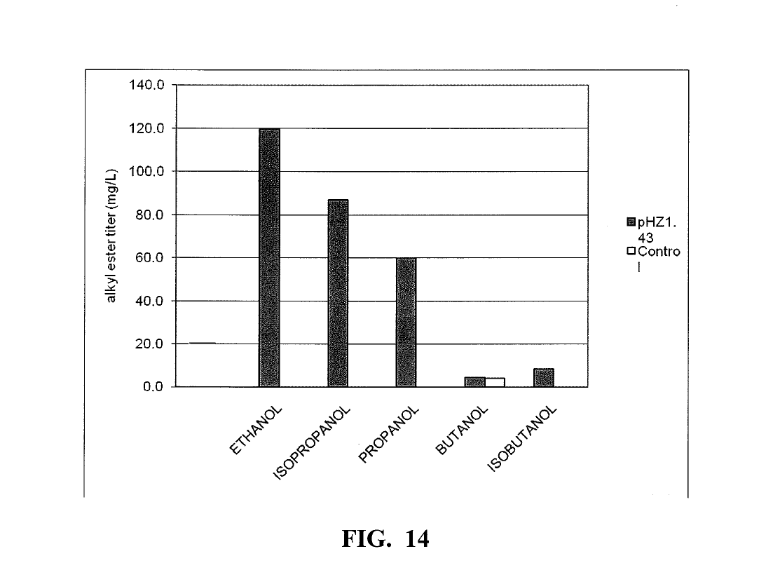

The results are shown in FIG. 14 which displays the total alkyl palmitate esters that resulted from various alkyl alcohol feeding, produced by C41(DE3)/pHZ1.43, with C41(DE3)/pCOLADuet-1, as the control. As shown in FIG. 14, all of the alcohols except those of butanol and 2-butanol resulted in alkyl esters. Thus, ester compositions can be modulated through selective addition of different alcohol moieties to the fermentation medium, even when a single production host is used.

### Example 20

### Plasmid Constructs for Fatty Ester Production in E. coli Hosts

For the production of fatty esters, additional plasmid constructs were generated, with each plasmid carrying all of the genes necessary for ester production in the form of a single operon under transcriptional control of the trc promoter. All genes were amplified using high fidelity Phusion™ polymerase (Finnzymes/NEB cat# F-530L). The truncated ′tesA gene was amplified from the plasmid pETDuet-1-′tesA. The fadD gene and adp1WS were amplified from pHZ1.61. The plasmid pHZ1.61, was constructed by replacing the NotI-AvrII fragment (carrying the acr1 gene) in the plasmid pCDFDuet-1-fadD-acr1 with the NotI-AvrII fragment from pHZ1.43 so that fadD and the ADP1 ester synthase were in one plasmid and both coding sequences were under the control of separate T7 promoters. The atfA1 gene was amplified from pHZ1.97-AtfA1, pCOLA-Duet-1 backbone with the atfA1 gene synthesized by DNA 2.0, cloned into NdeI and AvrII sites.

The operon was constructed using the pACYC-pTrc plasmid as a backbone. Plasmid pACYC-pTrc was constructed by PCR-amplifying the lacI^{q}, pTrc promoter and terminator region from pTrcHis2A (Invitrogen, Calrsbad, Calif.) using primers pTrc_F (5′TTTCGCGAGGCCGGCCCCGCCAACACCCGCTGACG) and pTrc_R (5′AAGGACGTCTTAATTAATCAGGAGAGCGTTCACCGACAA). The PCR product was then digested with AatII and NruI then cloned into pACYC177 digested with AatII and ScaI. The gene ′tesA was amplified using primers ′tesAForward (5′ ctctagaaataatttaactttaagtaggagauaggtacccatggcggacacgttattgat) and ′tesAReverse (5′ cttcgaattccatttaaattatttctagagtcattatgagtcatgatttactaaaggc). It was then cloned into the initial position of pACYC-pTrc using NcoI and EcoRI sites on both the insert and vector. T4 ligase (NEB cat# M0202S) was used for ligation of the vector and insert. Following overnight ligation, the DNA product was transformed into Top 10 one shot cells (Invitrogen cat# C4040-10). The ′tesA insertion into the pACYC ptrc vector was confirmed by restriction digestion. The amplification of ′tesA included sequence to create a SwaI restriction site at the 3′ end, as well as overlapping fragments for In-Fusion™ cloning (Clontech cat #631774).

Subsequent genes were cloned using In-Fusion™ cloning following linearization of the vector by overnight digestion with SwaI. The gene fadD was amplified using primers fadDForward (5′ ctctagaaataattttagttaagtataagaaggagatataccatggtgaagaaggtttggcttaa) and fadDReverse (5′ cttcgaattccatttaaattatttctagagttatcaggctttattgtccac). The PCR product was then cloned into the second position of the operon, following the ′tesA gene. This insertion of fadD was verified with restriction digestion. The insertion of fadD destroys the SwaI site following the ′tesA gene, but recreates the site at the 3′ end of fadD. This allows for another linearization of the vector by SwaI and subsequent In-Fusion™ cloning of the third gene atfA1 or adp1WS into the third and final position on the operon. AtfA1 was amplified with primers atfA1Forward (5′ ctctagaaataatttagttaagtataagaaggagatatacat) and atfA1Reverse (5′ cttcgaattccatttaaattatttctagagttactatttaattcctgcaccgatttcc), and adp1WS was amplified with primers adp1WSForward (5′ctctagaaataattttgtttaactttaagaaggagatataccatgggccgcccattacatccg) and adp1WSReverse (5′cttcgaattccatttaaattatttctagagagggctaatttagccctttagtttt). The proper insertion of the third gene was verified by restriction digestion. The resultant contructs were named pACYCop-adp1WS (for the plasmid carrying the operon with the adp1WS gene) and pACYCop-atfA1 (for the plasmid carrying the operon containing the atfA1 gene). The entire operon was removed from the plasmid by restriction digestion with MluI and EcoRI. It was then cloned into pOP-80 using the same restriction sites to generate the contructs pCLop-adp1WS and pCLop-atfA1 respectively.

pOP-80 was constructed by digesting the plasmid pCL1920 with the restriction enzymes AflII and SfoI (New England BioLabs Inc. Ipswich, Mass.). Three DNA sequence fragments were produced by this digestion. The 3737 bp fragment was gel-purified using a gel-purification kit (Qiagen, Inc. Valencia, Calif.). In parallel, a DNA sequence fragment containing the trc-promoter and lad region from the commercial plasmid pTrcHis2 (Invitrogen, Carlsbad, Calif.) was amplified by PCR using primers LF302 (5′-atatgacgtcGGCATCCGCTTACAGACA-3′) and LF303 (5′-aattcttaagTCAGGAGAGCGTTCACCGACAA-3′) introducing the recognition sites for the ZraI(gacgtc) and AflII(cttaag) enzymes, respectively. After amplification, the PCR products were purified using a PCR-purification kit (Qiagen, Inc. Valencia, Calif.) and digested with ZraI and AflII following the recommendations of the supplier (New England BioLabs Inc., Ipswich, Mass.). After digestion, the PCR product was gel-purified and ligated with the 3737 bp DNA sequence fragment derived from pCL1920 to generate the plasmid pOP-80.

### Example 21

### Production Host Construction

The present example describers a production host useful for the production of fatty esters, such as fatty acid methyl esters (FAME) or fatty acid ethyl esters (FAEE).

Construction of E. coli MG1655 (ΔfadE)

The fadE gene of E. coli MG1655 (an E. coli K strain) was deleted using the procedure described in Datsenko et al., Proc. Natl. Acad. Sci. USA 97: 6640-6645 (2000), with the following modifications described herein.

The two primers used to create the deletion were:

Del-fadE-F:
5′-AAAAACAGCAACAATGTGAGCTTTGTTGTAATTATATTGTAAAC
ATATTGATTCCGGGGATCCGTCGACC;
and
Del-fadE-R:
5′-AAACGGAGCCTTTCGGCTCCGTTATTCATTTACGCGGCTTCAAC
TTTCCTGTAGGCTGGAGCTGCTTC

The Del-fadE-F and Del-fadE-R primers each contain 50 bases of homology to the E. coli fadE gene and were used to amplify the Kanamycin resistance cassette from plasmid pKD13 by PCR as described in Datsenko et al., supra. The resulting PCR product was used to transform electrocompetent E. coli MG1655 cells containing pKD46 These cells were previously induced with arabinose for 3-4 h as described in Datsenko et al., supra. Following a 3 h outgrowth in SOC medium at 37° C., the cells were plated on Luria agar plates containing 50 μg/mL of Kanamycin. Resistant colonies were isolated after an overnight incubation at 37° C. Disruption of the fadE gene was confirmed in some of the colonies by PCR amplification using primers fadE-L2 and fadE-R1, which were designed to flank the fadE gene.

The fadE deletion confirmation primers used were:

| | |
|---|---|
| fadE-L2 | 5′-CGGGCAGGTGCTATGACCAGGAC; |
| and | |
| fadE-R1 | 5′-CGCGGCGTTGACCGGCAGCCTGG |

After the fadE deletion was confirmed, the Km^{R} marker was removed from one colony using the pCP20 plasmid as described in Datsenko et al., supra. The resulting MG1655 E. coli strain with the fadE gene deleted and the Km^{R} marker removed was named D1.

This example demonstrates the construction of a production host capable of producing fatty esters. This example further demonstrates an E. coli MG1655 ΔfadE.

### Example 22

### Comparison of Production of Fatty Acid Methyl Esters and Fatty Acid Ethyl Esters

The present example demonstrates a method for employing a single production host for the production of fatty acid methyl esters. The present example also compares the production fatty acid methyl esters and fatty acid ethyl esters.

E. coli strain MG1655 (ΔfadE) that has been transformed with plasmid pCLop-atfA1 was used to produce the described fatty esters. The plasmid pCLop-atfA1 is a pCL1920-based plasmid with ′tesA, fadD, and atfA1 under transcriptional control of the trc promoter, which is described herein.

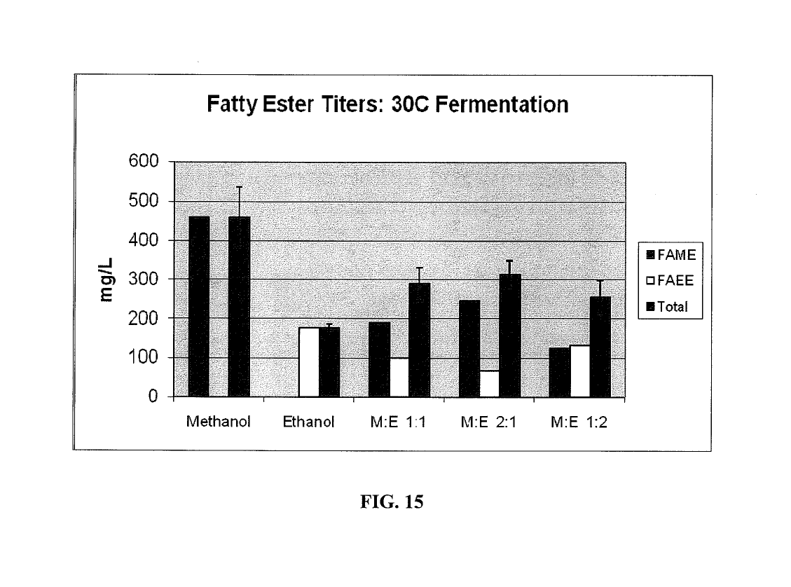

The E. coli strain MG1655 was cultured in Hu-9. Hu-9 is a M9 based minimal media supplemented with 2% glucose, 20 ug/mL uracil, and trace minerals. An overnight LB pre-seed culture was inoculated with either a single fresh colony or with a scraping from a frozen glycerol stock. The pre-seed culture was then used to inoculate an LB seed culture at a 1:100 dilution which was then allowed to grow at 37° C. until the OD₆₀₀=1.0-2.0. 2 mL of the seed culture was used to inoculate a 20 mL Hu-9 media production culture in a 125 mL shake flask. The production culture was allowed to grow at 37° C. with shaking until the OD₆₀₀=1.0. The cells were induced with 1 mM IPTG. The cultures were fed a 2% final volume of ethanol alone, methanol alone, or different ratios of methanol and ethanol and were fermented for 20 h at 30° C. GC/MS analysis showed that the total ester titers with methanol only feeding were higher than what was observed with ethanol only feeding or with the combined methanol and ethanol feeding (See, e.g., FIG. 15).

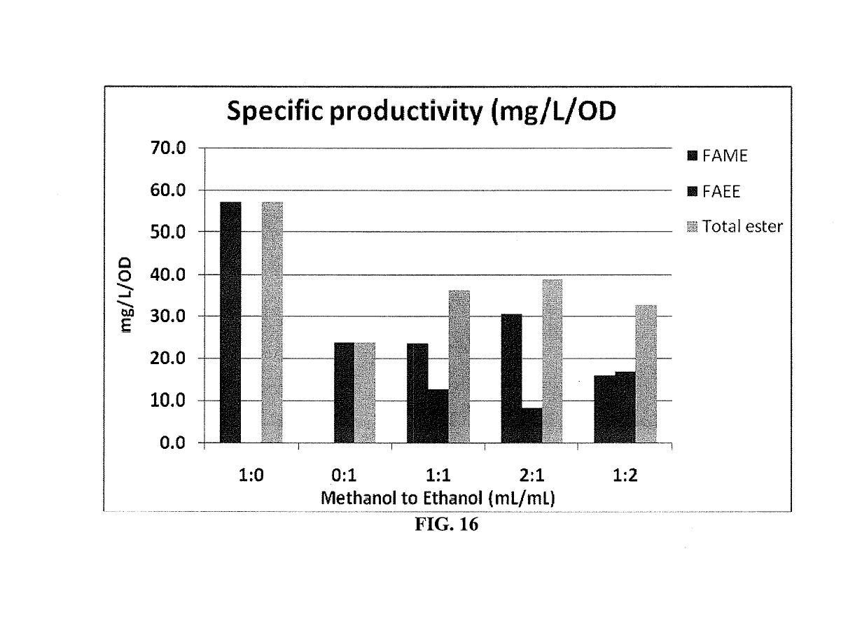

GC/MS analyses of the samples showed that AtfA1 can utilize methanol to make FAME, analogous to the way it produces FAEE using ethanol. Moreover, the amounts of fatty esters produced per unit volume of alcohol fed were higher for methanol than ethanol, suggesting a preference by AtfA1 for methanol as a substrate. It should be noted that both ethanol and methanol are known to exert toxic effects on the growth of E. coli above a certain concentration. Therefore, to determine the impact of this toxic effect, the ester values were normalized with OD₆₀₀ values to provide specific productivity (mg/L/OD), which would indicate any negative effects on growth (See, e.g., Table 35 and FIG. 16).

TABLE 35
Specific productivity of fatty ester production when fed
methanol and ethanol in various ratios.

| MeOH/EtOH | FAME/OD* | FAEE/OD* | total/OD* |
|---|---|---|---|
| 1:0 | 57.5 | 0.0 | 57.5 |
| 0:1 | 0.0 | 23.8 | 23.8 |
| 1:1 | 23.7 | 12.5 | 36.2 |
| 2:1 | 30.5 | 8.3 | 38.8 |
| 1:2 | 15.9 | 16.9 | 32.8 |

*all values in mg/L/OD

The present example demonstrates that substituting methanol with ethanol results in significant increases in specific productivities, (i.e., increased biodiesel production for the same volume of alcohol fed during fermentation). Moreover, in terms of economics, methanol is cheaper than ethanol. Therefore, productivity of esters per unit cost is also enhanced by using a methanol feed.

### Example 23

### Fatty Acid Methyl Ester Production Optimization

The present example demonstrates the optimization of methanol feeding to a fatty ester production host to produce FAME.

In previous examples, it was shown that only a small fraction of the total methanol fed (about 0.1% of the 2% fed) was utilized to produce FAME. Hence, the optimal level of methanol required to produce FAME was determined.

To identify the optimal volume of methanol required for the highest FAME production, fermentation experiments were carried out using the aforementioned fatty ester production host, but with different concentrations of methanol being fed at induction. The concentrations tested were: 0.1%, 0.3%, 0.5%, 1% and 2%. Pure methanol was diluted in water in the appropriate amounts so that the equivalent total volume of methanol or methanol+water was added to each of the production cultures. The cells were cultured in Hu-9 with 1% glucose using the standard fermentation protocol described herein with the 20 h of post-induction fermentation being carried out at 37° C.

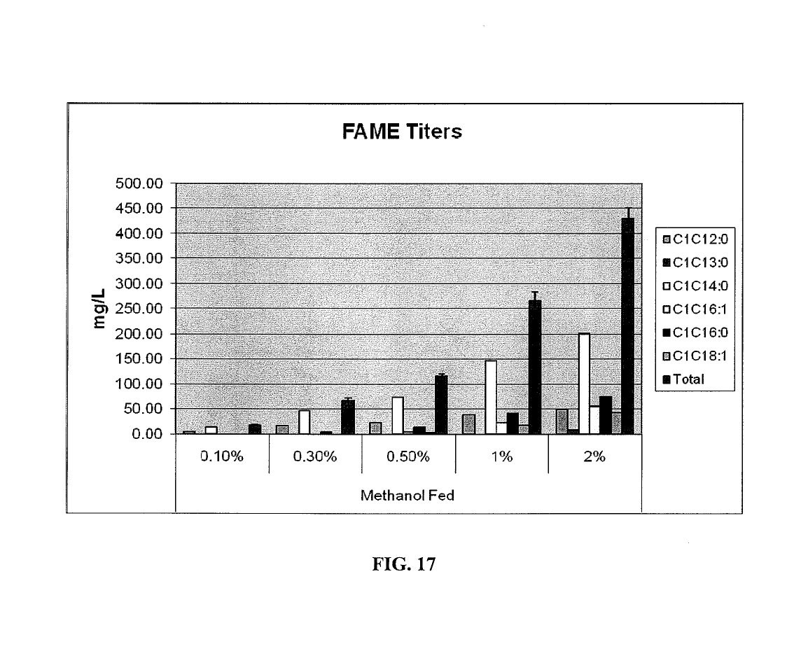

GC/MS analysis revealed that the amount of FAME produced is directly proportional to the amount of methanol fed to the cultures. Cultures fed 2% methanol produced around 430 mg/L total FAME while the cultures fed 0.1% methanol only produced about 20 mg/L (See, e.g., FIG. 17). In addition, free fatty acids were present in the extracts of cultures fed 0.1%-1% methanol. This indicates that for those experiments not enough methanol was present to pull the fatty acid substrates toward product formation. This resulted in an accumulation of free fatty acids. Hence, methanol could be the rate limiting reagent based on the reaction conditions provided. The reaction kinetics suggest that having an excess of fatty acids present in the reaction medium will generally result in additional amounts of methanol that are fed to the reaction medium to be synthesized into FAME products, although the processes herein are not intended to be limited by such theory.

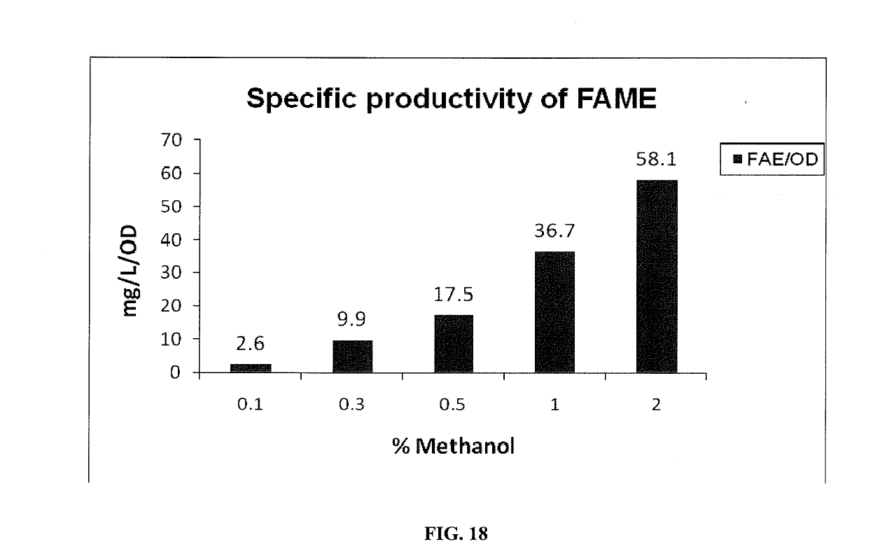

In addition to measuring FAME production, OD₆₀₀ measurements were taken to assess overall growth by the end of the 20 h fermentation run. The cultures fed 0.1% methanol grew to an average OD₆₀₀=6.6, cultures fed 0.3% methanol grew to OD₆₀₀=6.7, cultures fed 0.5% methanol grew to OD₆₀₀=6.6, cultures fed 1% methanol grew to OD₆₀₀=7.3, and cultures fed 2% methanol grew to OD₆₀₀=7.4. The cultures fed the higher amounts of methanol accumulated more cell mass than the cultures fed the lower volumes of methanol. These results indicate that for the amounts of methanol fed in this experiment, methanol supplementation does not appear to inhibit growth and that the higher volumes of methanol (e.g., 1% and 2%) seem to enhance growth slightly. Furthermore, the OD₆₀₀ measurements (as an indication of cell growth) were used to assess specific productivity for each of the reaction samples. The FAME production levels were normalized with OD₆₀₀ values to provide specific productivity (mg/L/OD). It appeared that almost proportionally, as methanol concentrations increased, the specific productivity increased by a similar level (See, e.g., Table 36, FIG. 18).

The present example demonstrates the optimal methanol concentration to feed the fatty ester production host in order to optimize production of FAME.

TABLE 36
Specific productivity data for cultures fed various
concentrations of methanol.

| % Methanol | FAME (mg/L) | OD | FAME/OD (mg/L/OD) |
|---|---|---|---|
| 0.1 | 17.1 | 6.6 | 2.6 |
| 0.3 | 66.4 | 6.7 | 9.9 |
| 0.5 | 115.6 | 6.6 | 17.5 |
| 1.0 | 266.7 | 7.3 | 36.7 |
| 2.0 | 430.8 | 7.4 | 58.1 |

### Example 24

### Fatty Acid Methyl Ester Production Optimization at Higher Methanol Concetrations

The present example demonstrates the optimization of methanol feeding to a fatty ester production host to produce FAME.

Higher methanol concentrations (e.g., greater than 2% levels) can increase fatty ester productivity of the fatty ester production host, but at the same time the culture growth may be adversely affected by the higher methanol concentrations. Hence, the net effect could be a decrease in fatty ester productivity. High methanol concentrations and their affects were examined in cultures that were fed methanol concentration from 2% to 6% of the total culture volume.

These experiments were carried out using the strain MG1655 (ΔfadE) with the pTrc-′tesA_fadD_atfA1 operon integrated onto the bacterial chromosome.

Integration of the PTrc-′tesA-fadD-atfA1 Operon into the E. coli MG1655 (ΔfadE) Chromosome at the lacI-lacZ Locus

Plasmid pCLop-atfA1 was digested with restriction enzyme HindIII. The chloramphenicol gene cassette was obtained from plasmid pLoxPcat2 (Genbank Accession No. AJ401047) by digestion with restriction enzymes BamHI and AvrII. Both DNA fragments were blunt-ended using the DNA polymerase Klenow fragment. The resulting fragments were ligated and transformed to generate plasmid pCLTFWcat.

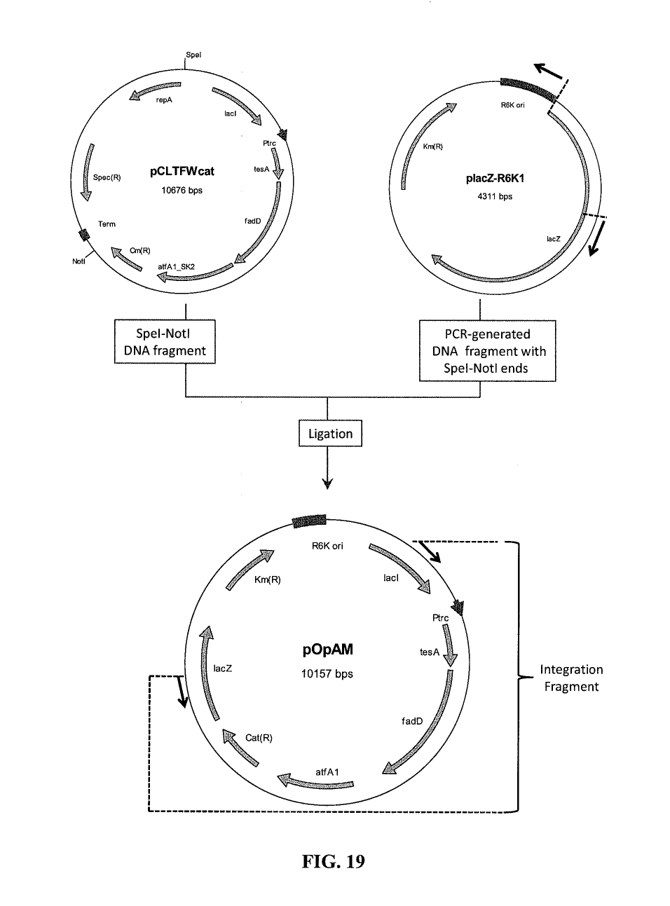

Plasmid placZ was used as a template for PCR amplification of the region shown in FIG. 19. placZ contains a 2249 bp DNA fragment from the E. coli lacZ gene (GenBank Accession Number: G1786539). placZ carries the R6K origin of replication and the Kanamycin antibiotic marker.

placZ has the following nucleotide sequence:

CTAGTAACGGCCGCCAGTGTGCTGGAATTCAGGCAGTTC
AACCTGTTGATAGTACGTACTAAGCTCTCATGTTTCACGTACTAAGCTCT
CATGTTTAACGTACTAAGCTCTCATGTTTAACGAACTAAACCCTCATGGC
TAACGTACTAAGCTCTCATGGCTAACGTACTAAGCTCTCATGTTTCACGT
ACTAAGCTCTCATGTTTGAACAATAAAATTAATATAAATCAGCAACTTAA
ATAGCCTCTAAGGTTTTAAGTTTTATAAGAAAAAAAAGAATATATAAGGC
TTTTAAAGCTTTTAAGGTTTAACGGTTGTGGACAACAAGCCAGGGATGTA
ACGCACTGAGAAGCCCTTAGAGCCTCTCAAAGCAATTTTCAGTGACACAG
GAACACTTAACGGCTGACAGCCTGAATTCTGCAGATCTGGCGTAATAGCG
AAGAGGCCCGCACCGATCGCCCTTCCCAACAGTTGCGCAGCCTGAATGGC
GAATGGCGCTTTGCCTGGTTTCCGGTACCAGAAGCGGTGCCGGAAAGCTG
GCTGGAGTGCGATCTTCCTGAGGCCGATACTGTCGTCGTCCCCTCAAACT
GGCAGATGCACGGTTACGATGCGCCCATCTACACCAACGTAACCTATCCC
ATTACGGTCAATCCGCCGTTTGTTCCCACGGAGAATCCGACGGGTTGTTA
CTCGCTCACATTTAATGTTGATGAAAGCTGGCTACAGGAAGGCCAGACGC
GAATTATTTTTGATGGCGTTAACTCGGCGTTTCATCTGTGGTGCAACGGG
CGCTGGGTCGGTTACGGCCAGGACAGTCGTTTGCCGTCTGAATTTGACCT
GAGCGCATTTTTACGCGCCGGAGAAAACCGCCTCGCGGTGATGGTGCTGC
GTTGGAGTGACGGCAGTTATCTGGAAGATCAGGATATGTGGCGGATGAGC
GGCATTTTCCGTGACGTCTCGTTGCTGCATAAACCGACTACACAAATCAG
CGATTTCCATGTTGCCACTCGCTTTAATGATGATTTCAGCCGCGCTGTAC
TGGAGGCTGAAGTTCAGATGTGCGGCGAGTTGCGTGACTACCTACGGGTA
ACAGTTTCTTTATGGCAGGGTGAAACGCAGGTCGCCAGCGGCACCGCGCC
TTTCGGCGGTGAAATTATCGATGAGCGTGGTGGTTATGCCGATCGCGTCA
CACTACGTCTGAACGTCGAAAACCCGAAACTGTGGAGCGCCGAAATCCCG
AATCTCTATCGTGCGGTGGTTGAACTGCACACCGCCGACGGCACGCTGAT
TGAAGCAGAAGCCTGCGATGTCGGTTTCCGCGAGGTGCGGATTGAAAATG
GTCTGCTGCTGCTGAACGGCAAGCCGTTGCTGATTCGAGGCGTTAACCGT
CACGAGCATCATCCTCTGCATGGTCAGGTCATGGATGAGCAGACGATGGT
GCAGGATATCCTGCTGATGAAGCAGAACAACTTTAACGCCGTGCGCTGTT
CGCATTATCCGAACCATCCGCTGTGGTACACGCTGTGCGACCGCTACGGC
CTGTATGTGGTGGATGAAGCCAATATTGAAACCCACGGCATGGTGCCAAT
GAATCGTCTGACCGATGATCCGCGCTGGCTACCGGCGATGAGCGAACGCG
TAACGCGAATGGTGCAGCGCGATCGTAATCACCCGAGTGTGATCATCTGG
TCGCTGGGGAATGAATCAGGCCACGGCGCTAATCACGACGCGCTGTATCG
CTGGATCAAATCTGTCGATCCTTCCCGCCCGGTGCAGTATGAAGGCGGCG
GAGCCGACACCACGGCCACCGATATTATTTGCCCGATGTACGCGCGCGTG
GATGAAGACCAGCCCTTCCCGGCTGTGCCGAAATGGTCCATCAAAAAATG
GCTTTCGCTACCTGGAGAGACGCGCCCGCTGATCCTTTGCGAATACGCCC
ACGCGATGGGTAACAGTCTTGGCGGTTTCGCTAAATACTGGCAGGCGTTT
CGTCAGTATCCCCGTTTACAGGGCGGCTTCGTCTGGGACTGGGTGGATCA
GTCGCTGATTAAATATGATGAAAACGGCAACCCGTGGTCGGCTTACGGCG
GTGATTTTGGCGATACGCCGAACGATCGCCAGTTCTGTATGAACGGTCTG
GTCTTTGCCGACCGCACGCCGCATCCAGCGCTGACGGAAGCAAAACACCA
GCAGCAGTTTTTCCAGTTCCGTTTATCCGGGCAAACCATCGAAGTGACCA
GCGAATACCTGTTCCGTCATAGCGATAACGAGCTCCTGCACTGGATGGTG
GCGCTGGATGGTAAGCCGCTGGCAAGCGGTGAAGTGCCTCTGGATGTCGC
TCCACAAGGTAAACAGTTGATTGAACTGCCTGAACTACCGCAGCCGGAGA
GCGCCGGGCAACTCTGGCTCACAGTACGCGTAGTGCAACCGAACGCGACC
GCATGGTCAGAAGCCGGGCACATCAGCGCCTGGCAGCAGTGGCGTCTGGC
GGAAAACCTCAGTGTGACGCTCCCCGCCGCGTCCCACGCCATCCCGCATC
TGACCACCAGCGAAATGGATTTTTGCATCGAGCTGGGTAATAAGCGTTGG
CAATTTAACCGCCAGTCAGGCTTTCTTTCACAGATGTGGATTGGCGATAA
AAAACAACTGCTGACGCCGCTGCGCGATCAGTTCACCCGTGCACGTCTGC
TGTCAGATAAAGTCTCCCGTGAACTTTACCCGGTGGTGCATATCGGGGAT
GAAAGCTGGCGCATGATGACCACCGATATGGCCAGTGTGCCGGTCTCCGT
TATCGGGGAAGAAGTGGCTGATCTCAGCCACCGCGAAAATGACATCAAAA
ACGCCATTAACCTGATGTTCTGGGGAATATAAATGTCAGGCATGAGATTA
TCAAAAAGGATCTTCACCTAGATCCTTTTCACGTAGAAAGCCAGTCCGCA
GAAACGGTGCTGACCCCGGATGAATGTCAGCTACTGGGCTATCTGGACAA
GGGAAAACGCAAGCGCAAAGAGAAAGCAGGTAGCTTGCAGTGGGCTTACA
TGGCGATAGCTAGACTGGGCGGTTTTATGGACAGCAAGCGAACCGGAATT
GCCAGCTGGGGCGCCCTCTGGTAAGGTTGGGAAGCCCTGCAAAGTAAACT
GGATGGCTTTCTCGCCGCCAAGGATCTGATGGCGCAGGGGATCAAGCTCT
GATCAAGAGACAGGATGAGGATCGTTTCGCATGATTGAACAAGATGGATT
GCACGCAGGTTCTCCGGCCGCTTGGGTGGAGAGGCTATTCGGCTATGACT
GGGCACAACAGACAATCGGCTGCTCTGATGCCGCCGTGTTCCGGCTGTCA
GCGCAGGGGCGCCCGGTTCTTTTTGTCAAGACCGACCTGTCCGGTGCCCT
GAATGAACTGCAAGACGAGGCAGCGCGGCTATCGTGGCTGGCCACGACGG
GCGTTCCTTGCGCAGCTGTGCTCGACGTTGTCACTGAAGCGGGAAGGGAC
TGGCTGCTATTGGGCGAAGTGCCGGGGCAGGATCTCCTGTCATCTCACCT
TGCTCCTGCCGAGAAAGTATCCATCATGGCTGATGCAATGCGGCGGCTGC
ATACGCTTGATCCGGCTACCTGCCCATTCGACCACCAAGCGAAACATCGC
ATCGAGCGAGCACGTACTCGGATGGAAGCCGGTCTTGTCGATCAGGATGA
TCTGGACGAAGAGCATCAGGGGCTCGCGCCAGCCGAACTGTTCGCCAGGC
TCAAGGCGAGCATGCCCGACGGCGAGGATCTCGTCGTGACCCATGGCGAT
GCCTGCTTGCCGAATATCATGGTGGAAAATGGCCGCTTTTCTGGATTCAT
CGACTGTGGCCGGCTGGGTGTGGCGGACCGCTATCAGGACATAGCGTTGG
CTACCCGTGATATTGCTGAAGAGCTTGGCGGCGAATGGGCTGACCGCTTC
CTCGTGCTTTACGGTATCGCCGCTCCCGATTCGCAGCGCATCGCCTTCTA
TCGCCTTCTTGACGAGTTCTTCTGAATTATTAACGCTTACAATTTCCTGA
TGCGGTATTTTCTCCTTACGCATCTGTGCGGTATTTCACACCGCATACAG
GTGGCACTTTTCGGGGAAATGTGCGCGGAACCCCTATTTGTTTATTTTTC
TAAATACATTCAAATATGTATCCGCTCATGAGACAATAACCCTGATAAAT
GCTTCAATAATAGCACGTGAGGAGGGCCACCATGGCCAAGTTGACCAGTG
CCGTTCCGGTGCTCACCGCGCGCGACGTCGCCGGAGCGGTCGAGTTCTGG
ACCGACCGGCTCGGGTTCTCCC

PCR primers LacZFnotI and pKDRspeI were designed to create restriction sites for the NotI and SpeI enzymes, respectively.

LacZfnotI
5′-
CAACCAGCGGCCGCGCAGACGATGGTGCAGGATATC
pKDRspeI
5′-
CCACACACTAGTCAGATCTGCAGAATTCAGGCTGTC

The resulting DNA fragment was ligated with a DNA fragment from plasmid pCLTFWcat digested with SpeI and NotI enzymes.

The ligation mixture was used as a template for another PCR reaction using primers lacIF and lacZR located on the lad and lacZ regions.

| | |
|---|---|
| lacIF | 5′-GGCTGGCTGGCATAAATATCTC |
| lacZR | 5′-CATCGCGTGGGCGTATTCG |

The resulting PCR product (“Integration Cassette”) contains approximately 500 bases of homology to lad or lacZ at each end. This PCR product was used to transform E. coli MG1655 ΔfadE cells that were made hypercompetent with plasmid pKD46 as described in Datsenko et al., supra.

The cells were cultured in M9 minimal media supplemented with 0.2M Bis-tris buffer, 5% glucose, and 1 g/L NH₄Cl using the fermentation protocol described herein with a 15 mL culture volume in a 125 mL baffled shake flask.

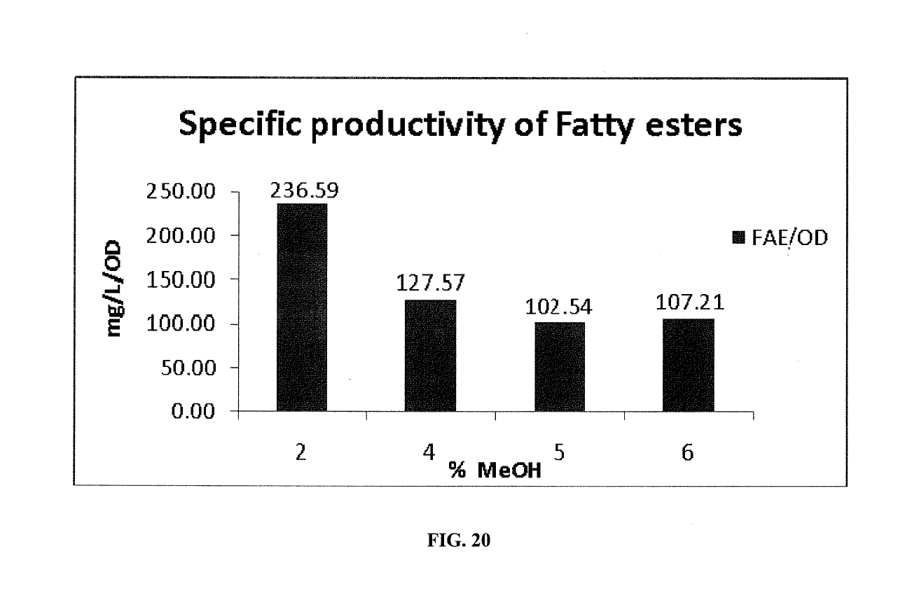

These conditions led to higher levels of growth and FAME titers compared to the previous results (See, e.g., Examples described herein). However, a relative comparison between specific productivities at 2% methanol feed vs. higher concentrations demonstrated that the highest total FAME titer was still achieved at 2% methanol (See, e.g., Table 37). Cultures fed 4%, 5%, and 6% methanol showed a steep drop in total titer as well as specific productivity at methanol concentrations ≧4% (v/v) (FIG. 20).

TABLE 37
Specific productivity data for cultures fed various
concentrations of methanol ≧2%.

| % Methanol | FAME (mg/L) | OD | FAME/OD (mg/L/OD) |
|---|---|---|---|
| 2.0 | 1632.5 | 6.9 | 236.6 |
| 4.0 | 483.5 | 3.8 | 127.6 |
| 5.0 | 323.0 | 3.2 | 102.5 |
| 6.0 | 282.5 | 2.6 | 107.2 |

The growth of MG1655 cells was reduced considerably as the amount of methanol increased. Compared to the culture growth at 2% methanol, the final culture growth in 4%, 5%, and 6% methanol samples was reduced by 45-63% (data based on OD₆₀₀ values.)

The present example demonstrates that substituting methanol with ethanol results in significant increases in specific productivities, (i.e., increased biodiesel production for the same volume of alcohol fed during fermentation). Moreover, in terms of economics, methanol is cheaper than ethanol. Therefore, productivity of esters per unit cost is also enhanced by using a methanol feed.

The present example demonstrates the optimal methanol concentration to feed the fatty ester production host in order to optimize production of FAME.

In this disclosure, the use of the singular can include the plural unless specifically stated otherwise or unless, as will be understood by one of skill in the art in light of the present disclosure, the singular is the only functional embodiment. Thus, for example, “a” can mean more than one, and “one embodiment” or “one example” can mean that the description applies to multiple embodiments. The phrase “and/or” denotes a shorthand way of indicating that the specific combination is contemplated in combination and, separately, in the alternative.

The section headings used herein are for organizational purposes only and are not to be construed as limiting the described subject matter in any way.

It will be appreciated that there is an implied “about” prior to the temperatures, concentrations, times, etc. discussed in the present teachings, such that slight and insubstantial deviations are within the scope of the present teachings herein. For example, “a primer” means that more than one primer can, but need not, be present. For example, but without limitation, one or more copies of a particular primer species, as well as one or more versions of a particular primer type, for example, but not limited to, a multiplicity of different forward primers can be present. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the invention.

### INCORPORATION BY REFERENCE

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application; including, but not limited to defined terms, term usage, described techniques, or the like, this application controls.

The following patent applications are incorporated by reference in their entirety: WO2007136762: Production of Fatty Acids and Derivatives Thereof, WO2008100251: Modified Microorganism Uses Therefor, WO2008113041: Process for Producing Low Molecular Weight Hydrocarbons from Renewable Resources, WO2008119082: Enhanced Production of Fatty Acid Derivatives, WO2009009391: Systems and Methods for the Production of Fatty Esters, and WO2009042950: Reduction of the Toxic Effect of Impurities From Raw Materials by Extractive Fermentation.

### EQUIVALENTS

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1-41. (canceled)

42. A genetically modified bacterial cell for production of a fatty ester, comprising an exogenous nucleic acid sequence encoding an alcohol forming polypeptide and an alcohol acetyltransferase polypeptide capable of catalyzing a conversion of a thioester and an alcohol, wherein said bacterial cell converts a renewable carbon source into a fatty ester.

43. The bacterial cell of claim 42, wherein said alcohol acetyltransferase polypeptide is alcohol acetyltransferase of EC 2.3.1.84.

44. The bacterial cell of claim 43, wherein said alcohol forming polypeptide is fatty alcohol forming acyl-CoA reductase of EC 1.1.1.*.

45. The bacterial cell of claim 44, wherein said cell is further genetically modified to express an exogenous nucleic acid sequence encoding acyl-CoA synthase.

46. The bacterial cell of claim 45, wherein said cell is further genetically modified to express an exogenous nucleic acid sequence encoding a thioesterase.

47. The bacterial cell of claim 42, wherein said alcohol forming polypeptide is acyl-CoA reductase of EC 1.1.1.50.

48. The bacterial cell of claim 47, wherein said cell is further genetically modified to express an exogenous nucleic acid sequence encoding acyl-CoA synthase.

49. The bacterial cell of claim 48, wherein said cell is further genetically modified to express an exogenous nucleic acid sequence encoding a thioesterase.

50. The bacterial cell of claim 49, wherein said cell is further genetically modified to express an exogenous nucleic acid sequence encoding an alcohol dehydrogenase of E.C. 1.1.1.1.

51. The bacterial cell of claim 42, wherein said bacterial cell is selected from the group consisting of Escherichia coli, Arthrobacter, Rhodotorula glutinins, Acinetobacter, Botryococcus braunii, Vibrio furnissii, Micrococcus leuteus, Stenotrophomonas maltophilia, Bacillus subtilis and cyanobacteria.

52. The bacterial cell of claim 42, wherein the fatty ester is released into a fermentation media.


SEQUENCE LISTING (25 sequences)

SEQ ID NO: 1 - DNA, 22 bp, Artificial Sequence
  Primer
tcatatgcgcccattacatccg

SEQ ID NO: 2 - DNA, 30 bp, Artificial Sequence
  Primer
tcctaggagggctaatttagccctttagtt

SEQ ID NO: 3 - DNA, 23 bp, Artificial Sequence
  Primer
tcatatggcgcccattacatccg

SEQ ID NO: 4 - DNA, 30 bp, Artificial Sequence; identical to SEQ ID NO: 2
  Primer

SEQ ID NO: 5 - DNA, 35 bp, Artificial Sequence
  Primer
tttcgcgaggccggccccgccaacacccgctgacg

SEQ ID NO: 6 - DNA, 39 bp, Artificial Sequence
  Primer
aaggacgtcttaattaatcaggagagcgttcaccgacaa

SEQ ID NO: 7 - DNA, 60 bp, Artificial Sequence
  Primer
ctctagaaataatttaactttaagtaggagauaggtacccatggcggacacgttattgat

SEQ ID NO: 8 - DNA, 58 bp, Artificial Sequence
  Primer
cttcgaattccatttaaattatttctagagtcattatgagtcatgatttactaaaggc

SEQ ID NO: 9 - DNA, 65 bp, Artificial Sequence
  Primer
ctctagaaataattttagttaagtataagaaggagatataccatggtgaagaaggtttggcttaa

SEQ ID NO: 10 - DNA, 51 bp, Artificial Sequence
  Primer
cttcgaattccatttaaattatttctagagttatcaggctttattgtccac

SEQ ID NO: 11 - DNA, 42 bp, Artificial Sequence
  Primer
ctctagaaataatttagttaagtataagaaggagatatacat

SEQ ID NO: 12 - DNA, 58 bp, Artificial Sequence
  Primer
cttcgaattccatttaaattatttctagagttactatttaattcctgcaccgatttcc

SEQ ID NO: 13 - DNA, 63 bp, Artificial Sequence
  Primer
ctctagaaataattttgtttaactttaagaaggagatataccatgggccgcccattacatccg

SEQ ID NO: 14 - DNA, 55 bp, Artificial Sequence
  Primer
cttcgaattccatttaaattatttctagagagggctaatttagccctttagtttt

SEQ ID NO: 15 - DNA, 28 bp, Artificial Sequence
  Primer
atatgacgtcggcatccgcttacagaca

SEQ ID NO: 16 - DNA, 32 bp, Artificial Sequence
  Primer
aattcttaagtcaggagagcgttcaccgacaa

SEQ ID NO: 17 - DNA, 70 bp, Artificial Sequence
  Primer
aaaaacagcaacaatgtgagctttgttgtaattatattgtaaacatattgattccggggatccgtcgacc

SEQ ID NO: 18 - DNA, 68 bp, Artificial Sequence
  Primer
aaacggagcctttcggctccgttattcatttacgcggcttcaactttcctgtaggctggagctgcttc

SEQ ID NO: 19 - DNA, 23 bp, Artificial Sequence
  Primer
cgggcaggtgctatgaccaggac

SEQ ID NO: 20 - DNA, 23 bp, Artificial Sequence
  Primer
cgcggcgttgaccggcagcctgg

SEQ ID NO: 21 - DNA, 36 bp, Artificial Sequence
  Primer
caaccagcggccgcgcagacgatggtgcaggatatc

SEQ ID NO: 22 - DNA, 36 bp, Artificial Sequence
  Primer
ccacacactagtcagatctgcagaattcaggctgtc

SEQ ID NO: 23 - DNA, 22 bp, Artificial Sequence
  Primer
ggctggctggcataaatatctc

SEQ ID NO: 24 - DNA, 19 bp, Artificial Sequence
  Primer
catcgcgtgggcgtattcg

SEQ ID NO: 25 - DNA, 4311 bp, Artificial Sequence
  placZ plasmid
ctagtaacggccgccagtgtgctggaattcaggcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttcagtgacacaggaacacttaacggctgacagcctgaattctgcagatctggcgtaatagcgaagaggcccgcaccgatcgcccttcccaacagttgcgcagcctgaatggcgaatggcgctttgcctggtttccggtaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctccc